(19) United States
(12) Patent Application Publication
(10) Pub. No.: US 2025/0188488 A1
(43) Pub. Date: Jun. 12, 2025

(54) VIRAL VECTOR COMPOSITIONS AND METHODS OF USE THEREOF

(71) Applicant: LogicBio Therapeutics, Inc., Lexington, MA (US)

(72) Inventors: Qiang Xiong, Newton, MA (US); B. Nelson Chau, Needham, MA (US)

(73) Assignee: LogicBio Therapeutics, Inc., Lexington, MA (US)

(21) Appl. No.: 18/288,658

(22) PCT Filed: Apr. 29, 2022
(86) PCT No.: PCT/US2022/026988

§ 371 Date: Oct. 27, 2023

Related U.S. Application Data
(60) Provisional application No. 63/182,738, filed on Apr. 30, 2021.

(51) Int. Cl. C12N 15/86; A61K 38/00; A61P 3/00; C12N 9/10; C12N 9/14; C12N 15/90

(52) U.S. Cl. CPC C12N 15/86; A61P 3/00; C12N 9/1051; C12N 9/14; C12N 15/907; A61K 38/00; C12N 2750/14143; C12Y 204/01017; C12Y 307/01002

(57) ABSTRACT

Presented herein are compositions and methods for improved gene editing with AAV vector constructs. Among other things, the present disclosure recognizes that homology arms of a certain length (e.g., at least 750 nt or at least 1000 nt each) may demonstrate improved editing activity. In some embodiments, the present disclosure recognizes that homology arms of a certain length (e.g., at least 750 nt or at least 1000 nt each) may demonstrate additional improvements in editing activity when homology arms are of different lengths.

### CROSS-REFERENCE TO RELATED APPLICATIONS

This application claims priority to U.S. Provisional Application No. 63/182,738 filed Apr. 30, 2021, the entirety of each of which is incorporated herein by reference.

### BACKGROUND

Genetic diseases caused by dysfunctional genes account for a large fraction of diseases worldwide. Gene therapy is emerging as a promising form of treatment aiming to mitigate the effects of genetic diseases

### SUMMARY

Prior to the present disclosure, certain AAV gene therapies that made use of homologous recombination (e.g., GENERIDE™) employed viral vector compositions comprising homology arms of the same length. Among other things, the present disclosure recognizes that homology arms of a certain length (e.g., at least 750 nt or at least 1000 nt each) may demonstrate improved editing activity. In some embodiments, the present disclosure recognizes that homology arms of a certain length (e.g., at least 750 nt or at least 1000 nt each) may demonstrate additional improvements in editing activity when homology arms are of different lengths.

Alternatively or additionally, the present disclosure further encompasses the recognition of a previously unidentified problem in vector design. In part, the present disclosure encompasses the recognition and observation that optimized vector designs, including specific homology arm lengths and/or ratios between the lengths of each homology arm, may be different between species. As demonstrated herein, optimal designs in one species or a model system for one species (e.g., wild-type mouse, humanized mouse, chimeric mouse) may differ from those in another species or a model system for another species (e.g., wild-type mouse, humanized mouse, chimeric mouse).

The present disclosure provides, inter alia, methods and technologies for improving the design and/or production of viral vectors, including AAV vectors. In accordance with various embodiments, the present disclosure provides an insight that certain sequence elements of viral vector constructs (e.g., homology arm sequences) may significantly impact gene editing efficiency. In some embodiments, the present disclosure demonstrates that administration of one or more viral vector compositions described herein (e.g., comprising balanced or unbalanced homology arms) at particular timepoints (e.g., postnatal timepoints) may improve editing efficiency. In some embodiments, administration timepoints are selected based upon target cell or tissue growth stages (e.g., corresponding to a particular age in one or more species).

In some embodiments, the present disclosure provides recombinant viral vectors, including at least one of a polynucleotide sequence encoding 1) first nucleic acid sequence and a second nucleic acid sequence, wherein the first nucleic acid wherein the first nucleic acid sequence comprises at least one exogenous gene sequence and the second nucleic acid sequence is positioned 5′ or 3′ to the first nucleic acid sequence and promotes the production of two independent gene products upon integration into a target integration site; 2) a third nucleic acid sequence positioned 5′ to the polynucleotide and comprising a sequence that is homologous to a genomic sequence 5′ of the target integration site; and 3) a fourth nucleic acid sequence positioned 3′ to the polynucleotide and comprising a sequence that is homologous to a genomic sequence 3′ of the target integration site, wherein each of the third nucleic acid sequence and fourth nucleic acid sequence are at least 1000 nt in length and wherein the third nucleic acid sequence and fourth nucleic acid sequence are different lengths. In some embodiments, provided viral vectors further include an exogenous gene sequence between 500 nt and 2500 nt in length. In some embodiments, provided viral vectors include an exogenous gene sequence between 1000 nt and 2000 nt in length. In some embodiments, provided viral vectors include a third nucleic acid sequence that is at least 750 nt in length. In some embodiments, provided viral vectors include a third nucleic acid sequence that is at least 1000 nt in length. In some embodiments, provided viral vectors include a third nucleic acid sequence that is at least 1600 nt in length. In some embodiments, provided viral vectors include a fourth nucleic acid sequence that is at least 750 nt in length. In some embodiments, provided viral vectors include a fourth nucleic acid sequence that is at least 1000 nt in length. In some embodiments, provided viral vectors include a fourth nucleic acid sequence that is at least 1600 nt in length. In some embodiments, provided viral vectors include a third and fourth nucleic acid sequence that are both at least 750 nt in length. In some embodiments, provided viral vectors include a third and fourth nucleic acid sequence that are both at least 1000 nt in length. In some embodiments, provided viral vectors include a third nucleic acid sequence that is 1000 nt in length and a fourth nucleic acid sequence that is 1600 nt in length. In some embodiments, provided viral vectors include a third nucleic acid sequence that is 1600 nt in length and a fourth nucleic acid sequence that is 1000 nt in length. In some embodiments, provided viral vectors are AAV vectors. In some embodiments, provided viral vectors are AAV vectors selected from AAV2, AAV8, AAV9, AAV-DJ, AAV-LK03, or AAV-sL65.

In some embodiments, provided compositions include one or more pharmaceutically acceptable excipients and recombinant viral vectors, which comprise at least one of a polynucleotide sequence encoding 1) first nucleic acid sequence and a second nucleic acid sequence, wherein the first nucleic acid wherein the first nucleic acid sequence comprises at least one exogenous gene sequence and the second nucleic acid sequence is positioned 5′ or 3′ to the first nucleic acid sequence and promotes the production of two independent gene products upon integration into a target integration site; 2) a third nucleic acid sequence positioned 5′ to the polynucleotide and comprising a sequence that is homologous to a genomic sequence 5′ of the target integration site; and 3) a fourth nucleic acid sequence positioned 3′ to the polynucleotide and comprising a sequence that is homologous to a genomic sequence 3′ of the target integration site, wherein each of the third nucleic acid sequence and fourth nucleic acid sequence are at least 1000 nt in length and wherein the third nucleic acid sequence and fourth nucleic acid sequence are different lengths.

In some embodiments, provided compositions are used in a method of integrating a transgene into the genome of one or more cells in a tissue in a subject, wherein said compositions are administered to a subject in which cells in the tissue fail to express a functional protein encoded by a gene product and the target integration site is in the genome of the one or more cells. In some embodiments, provided compositions may be administered in a method wherein the one or more cells are non-dividing cells. In some embodiments, provided compositions may be administered in a method wherein the one or more cells are liver, muscle, lung, or CNS cells. In some embodiments, provided compositions may be administered in a method during a postnatal period. In some embodiments, provided compositions may be administered in a method at least 7 days postnatal, at least 14 days postnatal, at least 21 days postnatal, or at least 28 days postnatal. In some embodiments, provided compositions may be administered in a method at or before 28 days postnatal. In some embodiments, provided compositions may be administered in a method wherein the transgene is or comprises UGT1A1, FAH, Factor IX, A1AT, ASL, or LIPA. In some embodiments, provided compositions may be administered in a method wherein integration efficiency is improved relative to a reference composition. In some embodiments, provided compositions may be administered in a method at a dosage of at least 5E13 vg/kg. In some embodiments, provided compositions may be administered in a method at a dosage of at least 1E14 vg/kg. In some embodiments, provided compositions may be administered in a method at a dosage of at least 2E14 vg/kg. In some embodiments, provided compositions may be administered in a method wherein the subject is an animal. In some embodiments, provided compositions may be administered in a method wherein the subject is a human. In some embodiments, provided compositions may be administered in a method wherein the subject has or is suspected to have Crigler-Najjar syndrome, tyrosinemia, hemophilia, alpha-1 antitrypsin deficiency, argininosuccinic aciduria, or lysosomal acid lipase deficiency.

### BRIEF DESCRIPTION OF THE DRAWING

FIG. 1 provides a flowchart of experimental protocols as well as a schematic illustrating certain components of mouse and human vectors tested in various experiments.

FIG. 2 demonstrates editing efficiency of various mouse GeneRide vectors comprising UGT1A1 transgene and homology arms of various lengths in C57BL/6 mice. (A) Fused mRNA levels (top left panel), levels of ALB-2A GeneRide biomarker (top right panel), and editing efficiency determined by percentage of stained hepatocytes (bottom left panel) were measured. The bottom right panel is a graphical representation of the correlation between the percentage of edited hepatocytes and the ALB-2A levels. (B) Mouse livers were harvested and stained to identify edited hepatocytes (brown spots, left image: representative IHC from liver treated with GeneRide vector with 1.0/1.0 KB homology arm (benchmark); right image: representative IHC from liver treated with GeneRide vector with 1.0/1.6 KB homology arm). Images were quantified using a semi-automatic algorithm to calculate the frequency of hepatocyte editing.

FIG. 3 demonstrates efficacy data for a single mouse GeneRide vector comprising a 5′ 1000 nt and a 3′ 1600 nt homology arm flanking a UGT1A1 transgene. Vectors were administered at various dosages (3E14 vg/kg, 1E14 vg/kg, 3E14 vg/kg) to neonatal (PND2), 14-day postnatal (PND14), and 28-day postnatal (PND28) UGT1−/− mice. Editing efficiency was measured through (A) assessment of UGT1A1 staining, (B) fused mRNA levels, and (C) ALB-2A biomarker levels. (D) Bilirubin levels (efficacy biomarker) and (E) percentage of Bilirubin reduction were also measured over the course of 12 weeks.

FIG. 4 depicts a graph of mouse liver weight relative to age. For liver-targeting gene therapies, mouse age may correspond with human age through assessment of respective percentage liver weight.

FIG. 5 depicts editing efficiency of various human GeneRide vectors comprising different homology arm lengths flanking a UGT1A1 transgene. Vectors were tested (A) in a human cell assay in vitro and (B) a liver-humanized mouse model in vivo (PXB). (C) PXB mice featured highly uniform liver humanization from transplanted human hepatocytes, as shown by IHC staining for human hepatocyte marker in PXB mouse liver.

FIG. 6 depicts editing activity of human and mouse GeneRide vectors comprising a UGT1A1 transgene. Tested vectors were previously optimized for activity in human (FIG. 5) or mouse (FIG. 2) systems.

FIG. 7 depicts editing activity of various mouse GeneRide vectors comprising different homology arm lengths flanking a human A1AT transgene tested in FvB wild-type mice. (A) Schematic of homology arm lengths for various vector designs. (B) ALB-2A biomarker and human A1AT (hA1AT) levels for various vector designs.

FIG. 8 depicts editing activity of various mouse GeneRide vectors comprising homology arms of the same length flanking a cyno-A1AT transgene tested in FvB wild-type mice. ALB-2A biomarker levels were measured for both designs.

FIG. 9 depicts editing activity of a single mouse GeneRide vector comprising a 5′ 1300 nt and a 3′ 1400 nt homology arm flanking a human UGT1A1 transgene. Vectors were administered at a dosage of 3E13 vg/kg to neonatal (PND1) and 14-day postnatal (PND14) C57B6 mice. Editing efficiency was measured through assessment of hUGT1A1 staining in mouse hepatocytes and levels of ALB-2A biomarker.

FIG. 10 demonstrates efficacy data for various mouse GeneRide vectors comprising a human UGT1A1 or a human Factor 9 (FIX) transgene. Vectors were administered to 28-day postnatal (PND28) mice. Editing efficiency was measured through assessment of (A) levels of ALB-2A biomarker (e.g., GeneRide biomarker), (B) transgene expression normalized to an actin reference, and (C) a comparison of transgene expression levels as compared to ALB-2A levels.

FIG. 11 shows efficacy data for various mouse GeneRide vectors comprising a human FIX transgene. Vectors were administered to mice to at various ages (neonatal, juvenile, and adult mice). Levels of ALB-2A biomarker were measured at various ages (neonatal, juvenile, and adult mice).

FIG. 12 demonstrates efficacy data for a single mouse GeneRide vector comprising a 5′ 1000 nt and a 3′ 1600 nt homology arm flanking a human FIX transgene. Vectors were administered to neonatal (PND2), 28-day postnatal (PND28), and 84-day postnatal (PND84) mice. Levels of ALB-2A biomarker and transgene expression were measured.

FIG. 13 shows results of treatment of mice with Hereditary Tyrosinemia with therapies as described herein. (A) shows an exemplary polynucleotide cassette with uneven homology arms. (B) shows an exemplary therapy time course. (C) shows assessment of circulating biomarker. (D) shows body weight changes of treated mice. (E) and (F) show levels of markers (e.g., alanine transaminase (ALT), Bilirubin) of liver function in treated mice. (G) shows immunohistochemical staining of liver samples with fumarylacetoacetate hydrolase (FAH) antibody. (H) shows assessment of integration into host genomic DNA (gDNA INT). Figure (I) shows assessment of presence of alfa-fetoprotein (AFP) which is a clinically validated in-life biomarker for hepatocellular carcinoma (HCC).

FIG. 14 shows results and selective advantage of treatment of mice with Hereditary Tyrosinemia with therapies as described herein. (A) shows an exemplary vector and treatment schedule. (B) shows assessment of ALB-2A biomarker (e.g., GeneRide biomarker). (C) shows survival rate of treated mice. (D) and (E) show markers of liver function (e.g., ALT, Succinylacetone (SUAC)) in treated mice.

FIG. 15 shows results of treatment of pediatric HT1 mice with therapies as described herein. (A) shows treatment time-course. (B) shows assessment of GENERIDE™ biomarkers. (C) shows body weight changes of treated mice. (D) shows alpha-fetoprotein (AFP) level in treated mice.

### DEFINITIONS

In order for the present invention to be more readily understood, certain terms are first defined below. Additional definitions for the following terms and other terms are set forth throughout the specification. The publications and other reference materials referenced herein to describe the background of the invention and to provide additional detail regarding its practice are hereby incorporated by reference.

The articles “a” and “an” are used herein to refer to one or to more than one (i.e., to at least one) of the grammatical object of the article. By way of example, “an element” means one element or more than one element.

About: The term “about” or “approximately”, when used herein in reference to a value, refers to a value that is similar, in context to the referenced value. In general, those skilled in the art, familiar with the context, will appreciate the relevant degree of variance encompassed by “about” in that context. For example, in some embodiments, the term “about” or “approximately” may encompass a range of values that within 25%, 20%, 19%, 18%, 17%, 16%, 15%, 14%, 13%, 12%, 11%, 10%, 9%, 8%, 7%, 6%, 5%, 4%, 3%, 2%, 1%, or less of the referred value.

Combination Therapy: As used herein, the term “combination therapy” refers to a clinical intervention in which a subject is simultaneously exposed to two or more therapeutic regimens (e.g. two or more therapeutic agents). In some embodiments, the two or more therapeutic regimens may be administered simultaneously. In some embodiments, the two or more therapeutic regimens may be administered sequentially (e.g., a first regimen administered prior to administration of any doses of a second regimen). In some embodiments, the two or more therapeutic regimens are administered in overlapping dosing regimens. In some embodiments, administration of combination therapy may involve administration of one or more therapeutic agents or modalities to a subject receiving the other agent(s) or modality. In some embodiments, combination therapy does not necessarily require that individual agents be administered together in a single composition (or even necessarily at the same time). In some embodiments, two or more therapeutic agents or modalities of a combination therapy are administered to a subject separately, e.g., in separate compositions, via separate administration routes (e.g., one agent orally and another agent intravenously), and/or at different time points. In some embodiments, two or more therapeutic agents may be administered together in a combination composition, or even in a combination compound (e.g., as part of a single chemical complex or covalent entity), via the same administration route, and/or at the same time.

Comparable: As used herein, the term “comparable” refers to two or more agents, entities, situations, sets of conditions, etc., that may not be identical to one another but that are sufficiently similar to permit comparison there between so that one skilled in the art will appreciate that conclusions may reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Comprising: A composition or method described herein as “comprising” one or more named elements or steps is open-ended, meaning that the named elements or steps are essential, but other elements or steps may be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and may also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step may be substituted for that element or step.

Corresponding to: As used herein, the term “corresponding to” may be used to designate the position/identity of a structural element in a compound or composition through comparison with an appropriate reference compound or composition. For example, in some embodiments, a monomeric residue in a polymer (e.g., an amino acid residue in a polypeptide or a nucleic acid residue in a polynucleotide) may be identified as “corresponding to” a residue in an appropriate reference polymer. For example, those of ordinary skill will appreciate that, for purposes of simplicity, residues in a polypeptide are often designated using a canonical numbering system based on a reference related polypeptide, so that an amino acid “corresponding to” a residue at position 190, for example, need not actually be the 190^{th} amino acid in a particular amino acid chain but rather corresponds to the residue found at 190 in the reference polypeptide; those of ordinary skill in the art readily appreciate how to identify “corresponding” amino acids. For example, those skilled in the art will be aware of various sequence alignment strategies, including software programs such as, for example, BLAST, CS-BLAST, CUSASW++, DIAMOND, FASTA, GGSEARCH/GLSEARCH, Genoogle, HMMER, HHpred/HHsearch, IDF, Infernal, KLAST, USEARCH, parasail, PSI-BLAST, PSI-Search, ScalaBLAST, Sequilab, SAM, SSEARCH, SWAPHI, SWAPHI-LS, SWIMM, or SWIPE that can be utilized, for example, to identify “corresponding” residues in polypeptides and/or nucleic acids in accordance with the present disclosure.

Engineered: In general, the term “engineered” refers to the aspect of having been manipulated by the hand of man. For example, a polynucleotide is considered to be “engineered” when two or more sequences, that are not linked together in that order in nature, are manipulated by the hand of man to be directly linked to one another in the engineered polynucleotide. For example, in some embodiments of the present invention, an engineered polynucleotide comprises a regulatory sequence that is found in nature in operative association with a first coding sequence but not in operative association with a second coding sequence, is linked by the hand of man so that it is operatively associated with the second coding sequence. Comparably, a cell or organism is considered to be “engineered” if it has been manipulated so that its genetic information is altered (e.g., new genetic material not previously present has been introduced, for example by transformation, mating, somatic hybridization, transfection, transduction, or other mechanism, or previously present genetic material is altered or removed, for example by substitution or deletion mutation, or by mating protocols). As is common practice and is understood by those in the art, progeny of an engineered polynucleotide or cell are typically still referred to as “engineered” even though the actual manipulation was performed on a prior entity.

Excipient: As used herein, refers to a non-therapeutic agent that may be included in a pharmaceutical composition, for example to provide or contribute to a desired consistency or stabilizing effect. In some embodiments, suitable pharmaceutical excipients may include, for example, starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Expression: As used herein, “expression” of a nucleic acid sequence refers to one or more of the following events: (1) production of an RNA template from a DNA sequence (e.g., by transcription); (2) processing of an RNA transcript (e.g., by splicing, editing, 5′ cap formation, and/or 3′ end formation); (3) translation of an RNA into a polypeptide or protein; and/or (4) post-translational modification of a polypeptide or protein.

Gene: As used herein, the term “gene” refers to a DNA sequence in a chromosome that encodes a gene product (e.g., an RNA product and/or a polypeptide product). In some embodiments, a gene includes a coding sequence (e.g., a sequence that encodes a particular gene product); in some embodiments, a gene includes a non-coding sequence. In some particular embodiments, a gene may include both coding (e.g., exonic) and non-coding (e.g., intronic) sequences. In some embodiments, a gene may include one or more regulatory elements (e.g. promoters, enhancers, silencers, termination signals) that, for example, may control or impact one or more aspects of gene expression (e.g., cell-type-specific expression, inducible expression).

Gene product or expression product: As used herein, the term “gene product” or “expression product” generally refers to an RNA transcribed from the gene (pre- and/or post-processing) or a polypeptide (pre- and/or post-modification) encoded by an RNA transcribed from the gene.

Homology: As used herein, the term “homology” refers to the overall relatedness between polymeric molecules, e.g., between polypeptide molecules. In some embodiments, polymeric molecules such as antibodies are considered to be “homologous” to one another if their sequences are at least 80%, 85%, 90%, 95%, or 99% identical. In some embodiments, polymeric molecules are considered to be “homologous” to one another if their sequences are at least 80%, 85%, 90%, 95%, or 99% similar.

Humanized mouse: As used herein, the term “humanized mouse” (also used interchangeably with “chimeric mouse”) refers to a mouse carrying functioning human genes, cells, tissues, and/or organs. In some embodiments, humanized mice may be used as a model system for humans (e.g., mice with humanized liver cells may be used as a model system for a human liver). In some embodiments, humanized mice have been transplanted with human cells and/or tissues. In some embodiments, humanized mice have been engineered to express human genes or gene products.

Identity: As used herein, the term “identity” refers to the overall relatedness between polymeric molecules, e.g., between nucleic acid molecules (e.g., DNA molecules and/or RNA molecules) and/or between polypeptide molecules. In some embodiments, polymeric molecules are considered to be “substantially identical” to one another if their sequences are at least 25%, 30%, 35%, 40%, 45%, 50%, 55%, 60%, 65%, 70%, 75%, 80%, 85%, 90%, 95%, or 99% identical. Calculation of the percent identity of two nucleic acid or polypeptide sequences, for example, can be performed by aligning the two sequences for optimal comparison purposes (e.g., gaps can be introduced in one or both of a first and a second sequences for optimal alignment and non-identical sequences can be disregarded for comparison purposes). In certain embodiments, the length of a sequence aligned for comparison purposes is at least 30%, at least 40%, at least 50%, at least 60%, at least 70%, at least 80%, at least 90%, at least 95%, or substantially 100% of the length of a reference sequence. The nucleotides at corresponding positions are then compared. When a position in the first sequence is occupied by the same residue (e.g., nucleotide or amino acid) as the corresponding position in the second sequence, then the molecules are identical at that position. The percent identity between the two sequences is a function of the number of identical positions shared by the sequences, taking into account the number of gaps, and the length of each gap, which needs to be introduced for optimal alignment of the two sequences. The comparison of sequences and determination of percent identity between two sequences can be accomplished using a mathematical algorithm. For example, the percent identity between two nucleotide sequences can be determined using the algorithm of Meyers and Miller (CABIOS, 1989, 4: 11-17), which has been incorporated into the ALIGN program (version 2.0). In some exemplary embodiments, nucleic acid sequence comparisons made with the ALIGN program use a PAM120 weight residue table, a gap length penalty of 12 and a gap penalty of 4. The percent identity between two nucleotide sequences can, alternatively, be determined using the GAP program in the GCG software package using an NWSgapdna.CMP matrix.

“Improved,” “increased” or “reduced”: As used herein, these terms, or grammatically comparable comparative terms, indicate values that are relative to a comparable reference measurement. For example, in some embodiments, an assessed value achieved with an agent of interest (e.g., a therapeutic agent) may be “improved” relative to that obtained with a comparable reference agent. Alternatively or additionally, in some embodiments, an assessed value achieved in a subject or system of interest may be “improved” relative to that obtained in the same subject or system under different conditions (e.g., prior to or after an event such as administration of an agent of interest), or in a different, comparable subject (e.g., in a comparable subject or system that differs from the subject or system of interest in presence of one or more indicators of a particular disease, disorder or condition of interest, or in prior exposure to a condition or agent, etc). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

In vitro: The term “in vitro” as used herein refers to events that occur in an artificial environment, e.g., in a test tube or reaction vessel, in cell culture, etc., rather than within a multi-cellular organism.

In vivo: as used herein refers to events that occur within a multi-cellular organism, such as a human and a non-human animal. In the context of cell-based systems, the term may be used to refer to events that occur within a living cell (as opposed to, for example, in vitro systems).

Marker: A marker, as used herein, refers to an entity or moiety whose presence or level is a characteristic of a particular state or event. In some embodiments, presence or level of a particular marker may be characteristic of presence or stage of a disease, disorder, or condition. To give but one example, in some embodiments, the term refers to a gene expression product that is characteristic of a particular tumor, tumor subclass, stage of tumor, etc. Alternatively or additionally, in some embodiments, a presence or level of a particular marker correlates with activity (or activity level) of a particular signaling pathway, for example that may be characteristic of a particular class of tumors. The statistical significance of the presence or absence of a marker may vary depending upon the particular marker. In some embodiments, detection of a marker is highly specific in that it reflects a high probability that the tumor is of a particular subclass. Such specificity may come at the cost of sensitivity (i.e., a negative result may occur even if the tumor is a tumor that would be expected to express the marker). Conversely, markers with a high degree of sensitivity may be less specific that those with lower sensitivity. According to the present invention a useful marker need not distinguish tumors of a particular subclass with 100% accuracy.

Nucleic acid: As used herein, in its broadest sense, refers to any compound and/or substance that is or can be incorporated into an oligonucleotide chain. In some embodiments, a nucleic acid is a compound and/or substance that is or can be incorporated into an oligonucleotide chain via a phosphodiester linkage. As will be clear from context, in some embodiments, “nucleic acid” refers to an individual nucleic acid residue (e.g., a nucleotide and/or nucleoside); in some embodiments, “nucleic acid” refers to an oligonucleotide chain comprising individual nucleic acid residues. In some embodiments, a “nucleic acid” is or comprises RNA; in some embodiments, a “nucleic acid” is or comprises DNA. In some embodiments, a nucleic acid is, comprises, or consists of one or more natural nucleic acid residues. In some embodiments, a nucleic acid is, comprises, or consists of one or more nucleic acid analogs. In some embodiments, a nucleic acid analog differs from a nucleic acid in that it does not utilize a phosphodiester backbone. For example, in some embodiments, a nucleic acid is, comprises, or consists of one or more “peptide nucleic acids”, which are known in the art and have peptide bonds instead of phosphodiester bonds in the backbone, are considered within the scope of the present invention. Alternatively or additionally, in some embodiments, a nucleic acid has one or more phosphorothioate and/or 5′-N-phosphoramidite linkages rather than phosphodiester bonds. In some embodiments, a nucleic acid is, comprises, or consists of one or more natural nucleosides (e.g., adenosine, thymidine, guanosine, cytidine, uridine, deoxyadenosine, deoxythymidine, deoxy guanosine, and deoxycytidine). In some embodiments, a nucleic acid is, comprises, or consists of one or more nucleoside analogs (e.g., 2-aminoadenosine, 2-thiothymidine, inosine, pyrrolo-pyrimidine, 3-methyl adenosine, 5-methylcytidine, C-5 propynyl-cytidine, C-5 propynyl-uridine, 2-aminoadenosine, C5-bromouridine, C5-fluorouridine, C5-iodouridine, C5-propynyl-uridine, C5-propynyl-cytidine, C5-methylcytidine, 2-aminoadenosine, 7-deazaadenosine, 7-deazaguanosine, 8-oxoadenosine, 8-oxoguanosine, 0(6)-methylguanine, 2-thiocytidine, methylated bases, intercalated bases, and combinations thereof). In some embodiments, a nucleic acid comprises one or more modified sugars (e.g., 2′-fluororibose, ribose, 2′-deoxyribose, arabinose, and hexose) as compared with those in natural nucleic acids. In some embodiments, a nucleic acid has a nucleotide sequence that encodes a functional gene product such as an RNA or protein. In some embodiments, a nucleic acid includes one or more introns. In some embodiments, nucleic acids are prepared by one or more of isolation from a natural source, enzymatic synthesis by polymerization based on a complementary template (in vivo or in vitro), reproduction in a recombinant cell or system, and chemical synthesis. In some embodiments, a nucleic acid is at least 3, 4, 5, 6, 7, 8, 9, 10, 15, 20, 25, 30, 35, 40, 45, 50, 55, 60, 65, 70, 75, 80, 85, 90, 95, 100, 110, 120, 130, 140, 150, 160, 170, 180, 190, 20, 225, 250, 275, 300, 325, 350, 375, 400, 425, 450, 475, 500, 600, 700, 800, 900, 1000, 1500, 2000, 2500, 3000, 3500, 4000, 4500, 5000 or more residues long. In some embodiments, a nucleic acid is partly or wholly single stranded; in some embodiments, a nucleic acid is partly or wholly double stranded. In some embodiments a nucleic acid has a nucleotide sequence comprising at least one element that encodes, or is the complement of a sequence that encodes, a polypeptide. In some embodiments, a nucleic acid has enzymatic activity.

Peptide: The term “peptide” as used herein refers to a polypeptide that is typically relatively short, for example having a length of less than about 100 amino acids, less than about 50 amino acids, less than about 40 amino acids less than about 30 amino acids, less than about 25 amino acids, less than about 20 amino acids, less than about 15 amino acids, or less than 10 amino acids.

Pharmaceutically acceptable carrier: As used herein, the term “pharmaceutically acceptable carrier” means a pharmaceutically-acceptable material, composition or vehicle, such as a liquid or solid filler, diluent, excipient, or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the subject. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; pH buffered solutions; polyesters, polycarbonates and/or polyanhydrides; and other non-toxic compatible substances employed in pharmaceutical formulations.

Pharmaceutical composition: As used herein, the term “pharmaceutical composition” refers to an active agent, formulated together with one or more pharmaceutically acceptable carriers. In some embodiments, active agent is present in unit dose amount appropriate for administration in a therapeutic regimen that shows a statistically significant probability of achieving a predetermined therapeutic effect when administered to a relevant population. In some embodiments, pharmaceutical compositions may be specially formulated for administration in solid or liquid form, including those adapted for the following: oral administration, for example, drenches (aqueous or non-aqueous solutions or suspensions), tablets, e.g., those targeted for buccal, sublingual, and systemic absorption, boluses, powders, granules, pastes for application to the tongue; parenteral administration, for example, by subcutaneous, intramuscular, intravenous or epidural injection as, for example, a sterile solution or suspension, or sustained-release formulation; topical application, for example, as a cream, ointment, or a controlled-release patch or spray applied to the skin, lungs, or oral cavity; intravaginally or intrarectally, for example, as a pessary, cream, or foam; sublingually; ocularly; transdermally; or nasally, pulmonary, and to other mucosal surfaces.

Polypeptide: The term “polypeptide”, as used herein, generally has its art-recognized meaning of a polymer of at least three amino acids. Those of ordinary skill in the art will appreciate that the term “polypeptide” is intended to be sufficiently general as to encompass not only polypeptides having a complete sequence recited herein, but also to encompass polypeptides that represent functional fragments (i.e., fragments retaining at least one activity) of such complete polypeptides. Moreover, those of ordinary skill in the art understand that protein sequences generally tolerate some substitution without destroying activity. Thus, any polypeptide that retains activity and shares at least about 30-40% overall sequence identity, often greater than about 50%, 60%, 70%, or 80%, and further usually including at least one region of much higher identity, often greater than 90% or even 95%, 96%, 97%, 98%, or 99% in one or more highly conserved regions, usually encompassing at least 3-4 and often up to 20 or more amino acids, with another polypeptide of the same class, is encompassed within the relevant term “polypeptide” as used herein. Polypeptides may contain L-amino acids, D-amino acids, or both and may contain any of a variety of amino acid modifications or analogs known in the art. Useful modifications include, e.g., terminal acetylation, amidation, methylation, etc. In some embodiments, proteins may comprise natural amino acids, non-natural amino acids, synthetic amino acids, and combinations thereof. The term “peptide” is generally used to refer to a polypeptide having a length of less than about 100 amino acids, less than about 50 amino acids, less than 20 amino acids, or less than 10 amino acids. In some embodiments, proteins are antibodies, antibody fragments, biologically active portions thereof, and/or characteristic portions thereof.

Prevent or prevention: as used herein when used in connection with the occurrence of a disease, disorder, and/or condition, refers to reducing the risk of developing the disease, disorder and/or condition and/or to delaying onset of one or more characteristics, signs, or symptoms of the disease, disorder or condition. Prevention may be considered complete when onset of a disease, disorder or condition has been delayed for a predefined period of time.

Risk: as will be understood from context, “risk” of a disease, disorder, and/or condition refers to a likelihood that a particular individual will develop the disease, disorder, and/or condition. In some embodiments, risk is expressed as a percentage. In some embodiments, risk is from 0, 1, 2, 3, 4, 5, 6, 7, 8, 9, 10, 20, 30, 40, 50, 60, 70, 80, 90 up to 100%. In some embodiments risk is expressed as a risk relative to a risk associated with a reference sample or group of reference samples. In some embodiments, a reference sample or group of reference samples have a known risk of a disease, disorder, condition and/or event. In some embodiments a reference sample or group of reference samples are from individuals comparable to a particular individual. In some embodiments, relative risk is 0, 1, 2, 3, 4, 5, 6, 7, 8, 9, 10, or more.

Subject: As used herein, the term “subject” refers an organism, typically a mammal (e.g., a human, in some embodiments including prenatal human forms). In some embodiments, a subject is suffering from a relevant disease, disorder or condition. In some embodiments, a subject is susceptible to a disease, disorder, or condition. In some embodiments, a subject displays one or more symptoms or characteristics of a disease, disorder or condition. In some embodiments, a subject does not display any symptom or characteristic of a disease, disorder, or condition. In some embodiments, a subject is someone with one or more features characteristic of susceptibility to or risk of a disease, disorder, or condition. In some embodiments, a subject is a patient. In some embodiments, a subject is an individual to whom diagnosis and/or therapy is and/or has been administered.

Substantially: As used herein, the term “substantially” refers to the qualitative condition of exhibiting total or near-total extent or degree of a characteristic or property of interest. One of ordinary skill in the biological arts will understand that biological and chemical phenomena rarely, if ever, go to completion and/or proceed to completeness or achieve or avoid an absolute result. The term “substantially” is therefore used herein to capture the potential lack of completeness inherent in many biological and chemical phenomena.

Susceptible to: An individual who is “susceptible to” a disease, disorder, and/or condition is one who has a higher risk of developing the disease, disorder, and/or condition than does a member of the general public. In some embodiments, an individual who is susceptible to a disease, disorder and/or condition may not have been diagnosed with the disease, disorder, and/or condition. In some embodiments, an individual who is susceptible to a disease, disorder, and/or condition may exhibit symptoms of the disease, disorder, and/or condition. In some embodiments, an individual who is susceptible to a disease, disorder, and/or condition may not exhibit symptoms of the disease, disorder, and/or condition. In some embodiments, an individual who is susceptible to a disease, disorder, and/or condition will develop the disease, disorder, and/or condition. In some embodiments, an individual who is susceptible to a disease, disorder, and/or condition will not develop the disease, disorder, and/or condition.

Therapeutic agent: As used herein, the phrase “therapeutic agent” refers to an agent that, when administered to a subject, has a therapeutic effect and/or elicits a desired biological and/or pharmacological effect. In some embodiments, a therapeutic agent is any substance that can be used to alleviate, ameliorate, relieve, inhibit, prevent, delay onset of, reduce severity of, and/or reduce incidence of one or more symptoms or features of a disease, disorder, and/or condition.

Treatment: As used herein, the term “treatment” (also “treat” or “treating”) refers to administration of a therapy that partially or completely alleviates, ameliorates, relives, inhibits, delays onset of, reduces severity of, and/or reduces incidence of one or more signs, symptoms, features, and/or causes of a particular disease, disorder, and/or condition. In some embodiments, such treatment may be of a subject who does not exhibit signs of the relevant disease, disorder and/or condition and/or of a subject who exhibits only early signs of the disease, disorder, and/or condition. Alternatively or additionally, such treatment may be of a subject who exhibits one or more established signs of the relevant disease, disorder and/or condition. In some embodiments, treatment may be of a subject who has been diagnosed as suffering from the relevant disease, disorder, and/or condition. In some embodiments, treatment may be of a subject known to have one or more susceptibility factors that are statistically correlated with increased risk of development of the relevant disease, disorder, and/or condition. Thus, in some embodiments, treatment may be prophylactic; in some embodiments, treatment may be therapeutic.

Variant: As used herein, the term “variant” refers to an entity that shows significant structural identity with a reference entity but differs structurally from the reference entity in the presence or absence or in the level of one or more chemical moieties as compared with the reference entity. In some embodiments, a variant also differs functionally from its reference entity. In general, whether a particular entity is properly considered to be a “variant” of a reference entity is based on its degree of structural identity with the reference entity. As will be appreciated by those skilled in the art, any biological or chemical reference entity has certain characteristic structural elements. A variant, by definition, is a distinct chemical entity that shares one or more such characteristic structural elements. To give but a few examples, a small molecule may have a characteristic core structural element (e.g., a macrocycle core) and/or one or more characteristic pendent moieties so that a variant of the small molecule is one that shares the core structural element and the characteristic pendent moieties but differs in other pendent moieties and/or in types of bonds present (single vs double, E vs Z, etc) within the core, a polypeptide may have a characteristic sequence element comprised of a plurality of amino acids having designated positions relative to one another in linear or three-dimensional space and/or contributing to a particular biological function, a nucleic acid may have a characteristic sequence element comprised of a plurality of nucleotide residues having designated positions relative to on another in linear or three-dimensional space. In some embodiments, a variant polypeptide or nucleic acid may differ from a reference polypeptide or nucleic acid as a result of one or more differences in amino acid or nucleotide sequence and/or one or more differences in chemical moieties (e.g., carbohydrates, lipids, phosphate groups) that are covalently components of the polypeptide or nucleic acid (e.g., that are attached to the polypeptide or nucleic acid backbone). In some embodiments, a variant polypeptide or nucleic acid shows an overall sequence identity with a reference polypeptide or nucleic acid that is at least 85%, 86%, 87%, 88%, 89%, 90%, 91%, 92%, 93%, 94%, 95%, 96%, 97%, or 99%. Alternatively or additionally, in some embodiments, a variant polypeptide or nucleic acid does not share at least one characteristic sequence element with a reference polypeptide or nucleic acid. In some embodiments, a reference polypeptide or nucleic acid has one or more biological activities. In some embodiments, a variant polypeptide or nucleic acid shares one or more of the biological activities of the reference polypeptide or nucleic acid. In some embodiments, a variant polypeptide or nucleic acid lacks one or more of the biological activities of the reference polypeptide or nucleic acid. In some embodiments, a variant polypeptide or nucleic acid shows a reduced level of one or more biological activities as compared to the reference polypeptide or nucleic acid. In some embodiments, a polypeptide or nucleic acid of interest is considered to be a “variant” of a parent or reference polypeptide or nucleic acid if it has an amino acid or nucleotide sequence that is identical to that of the reference but for a small number of sequence alterations at particular positions. Typically, fewer than about 20%, about 15%, about 10%, about 9%, about 8%, about 7%, about 6%, about 5%, about 4%, about 3%, or about 2% of the residues in a variant are substituted, inserted, or deleted, as compared to the reference. In some embodiments, a variant polypeptide or nucleic acid comprises about 10, about 9, about 8, about 7, about 6, about 5, about 4, about 3, about 2, or about 1 substituted residue(s) as compared with a reference. Often, a variant polypeptide or nucleic acid comprises a very small number (e.g., fewer than about 5, about 4, about 3, about 2, or about 1) number of substituted, inserted, or deleted, functional residues (i.e., residues that participate in a particular biological activity) relative to the reference. In some embodiments, a variant polypeptide or nucleic acid comprises not more than about 5, about 4, about 3, about 2, or about 1 addition(s) or deletion(s), and, in some embodiments, comprises no additions or deletions, as compared to the reference. In some embodiments, a variant polypeptide or nucleic acid comprises fewer than about 25, about 20, about 19, about 18, about 17, about 16, about 15, about 14, about 13, about 10, about 9, about 8, about 7, about 6, and commonly fewer than about 5, about 4, about 3, or about 2 additions or deletions as compared to the reference. In some embodiments, a reference polypeptide or nucleic acid is one found in nature. In some embodiments, a reference polypeptide or nucleic acid is a human polypeptide or nucleic acid.

### DETAILED DESCRIPTION OF CERTAIN EMBODIMENTS

### Gene Therapy

Genetic diseases caused by dysfunctional genes have been reported to account for nearly 80% of approximately 7,136 diseases reported as of 2019 (See, Genetic and rare Diseases Information Center and Global Genes). More than 330 million people worldwide are affected by a genetic disease, and almost half of these cases are estimated to be children. However, only about 500 human diseases are estimated to be treatable with available drugs, indicating that new therapies and options for treatment are necessary to address a substantial proportion of these genetic disorders. Gene therapy is an emerging form of treatment that aims to mediate the effects of genetic disorders through transmission of genetic material into a subject. In some embodiments, gene therapy may comprise transcription and/or translation of transferred genetic material, and/or by integration of transferred genetic material into a host genome through administration of nucleic acids, viruses, or genetically engineered microorganisms (See, FDA Guidelines). Gene therapy can allow delivery of therapeutic genetic material to any specific cell, tissue, and/or organ of a subject for treatment. In some embodiments, gene therapy involves transfer of a therapeutic gene, or transgene, to a host cell.

### Viral Gene Therapy

Viruses have emerged as an appealing vehicle for gene therapy due to their ability to express high levels of a payload (e.g., a transgene) and, in some embodiments, their ability to stably express a payload (e.g., transgene) within a host's genome. Recombinant AAVs are popular viral vectors for gene therapy, as they often produce high viral yields, mild immune response, and are able to infect different cell types.

In conventional AAV gene therapy, rAAVs can be engineered to deliver therapeutic payloads (e.g., transgenes) to target cells without integrating into chromosomal DNA. One or more payloads (e.g., transgenes) may be expressed from a non-integrated genetic element called an episome that exists within the cell nucleus. Although conventional gene therapy may be effective in initially transduced cells, episomal expression is transient and gradually decreases over time, inter alia, with cell turnover. For cells with a longer lifespan (e.g., cells that exist for a significant portion of a subject's lifetime), episomal expression can be effective. However, conventional gene therapy can have drawbacks when applied to a subject early in life (e.g., during childhood), as rapid tissue growth during development can result in dilution and eventual loss of therapeutic benefit of a payload (e.g., transgene).

A second type of AAV gene therapy, GENERIDE™, harnesses homologous recombination (HR), a naturally occurring DNA repair process that maintains the fidelity of a cell genome. GENERIDE™ uses HR to insert one or more payloads (e.g., transgenes) into specific target loci within a genomic sequence. In some embodiments, GENERIDE™ makes use of endogenous promoters at one or more target loci to drive high levels of tissue-specific expression. GENERIDE™ does not require use of exogenous nucleases or promoters, thereby reducing detrimental effects often associated with these elements. Furthermore, GENERIDE™ platform technology has potential to overcome some of the key limitations of both traditional gene therapy and conventional gene editing approaches in a way that is well positioned to treat genetic diseases, particularly in pediatric subjects. GENERIDE™ uses an AAV vector to deliver a gene into the nucleus of the cell. It then uses HR to stably integrate a corrective gene into the genome of a subject at a location where it is regulated by an endogenous promoter, allowing lifelong protein production, even as the body grows and changes over time, which is not feasible with conventional AAV gene therapy.

Previous work on non-disruptive gene targeting is described in WO 2013/158309, incorporated herein by reference. Previous work on genome editing without nucleases is described in WO 2015/143177, incorporated herein by reference. Previous work on non-disruptive gene therapy for the treatment of MMA is described in WO 2020/032986, incorporated herein by reference. Previous work on monitoring of gene therapy is described in WO/2020/214582, incorporated herein by reference.

### Viral Structure and Function

### Viral Vectors

Viral vectors comprise virus or viral chromosomal material, within which a heterologous nucleic acid sequence can be inserted for transfer into a target sequence of interest (e.g., for transfer into genomic DNA within a cell). Various viruses can be used as viral vectors, including, e.g., single-stranded DNA (ssDNA), double-stranded DNA (dsDNA) viruses, and/or RNA viruses with a DNA stage in their lifecycle. In some embodiments, a viral vector is or comprises an adeno-associated virus (AAV) or AAV variant.

In some embodiments, a vector particle is a single unit of virus comprising a capsid encapsidating a virus-based polynucleotide (e.g., a wild-type viral genome or a recombinant viral vector). In some embodiments, a vector particle is or comprises an AAV vector particle. In some embodiments, an AAV vector particle refers to a vector particle comprised of at least one AAV capsid protein and an encapsidated AAV vector. In some embodiments, a vector particle (also referred to as a viral vector) comprises at least one AAV capsid protein and an encapsidated AAV vector, wherein the vector further comprises one or more heterologous polynucleotide sequences.

### Capsid Proteins

In some embodiments, an expression construct comprises polynucleotide sequences encoding capsid proteins from one or more AAV subtypes, including naturally occurring and recombinant AAVs. In some embodiments, an expression construct comprises polynucleotide sequences encoding capsid proteins from AAV1, AAV2, AAV3, AAV4, AAV5, AAV6, AAV7, AAV8, AAV9, AAV10, AAV11, AAVC11.01, AAVC11.02, AAVC11.03, AAVC11.04, AAVC11.05, AAVC11.06, AAVC11.07, AAVC11.08, AAVC11.09, AAVC11.10, AAVC11.11 (referred to interchangeably herein as sL65), AAVC11.12, AAVC11.13, AAVC11.14, AAVC11.15, AAVC11.16, AAVC11.17, AAVC11.18, AAVC11.19, AAV-DJ, AAV-LK03, AAV-LK19, AAVrh.74, AAVrh.10, AAVhu.37, AAVrh.K, AAVrh.39, AAV12, AAV 13, AAVrh.8, avian AAV, bovine AAV, canine AAV, equine AAV, primate AAV, non-primate AAV, ovine AAV, a hybrid AAV (e.g., an AAV comprising one more sequences of one AAV subtype and one or more sequences of a second subtype), and/or an AAV comprising a mutant AAV capsid protein or a chimeric AAV capsid (e.g., a capsid with polynucleotide sequences derived from two or more different serotypes of AAV), or variants thereof.

In some embodiments, viral vectors are packaged within capsid proteins (e.g., capsid proteins from one or more AAV subtypes). In some embodiments, capsid proteins provide increased or enhanced transduction of cells (e.g., human or murine cells) relative to a reference capsid protein. In some embodiments, capsid proteins provide increased or enhanced transduction of certain cells or tissue types (e.g., liver tropism, muscle tropism, CNS tropism, lung tropism) relative to a reference capsid protein. In some embodiments, capsid proteins increase or enhance transduction of cells or tissues (e.g., liver, muscle, and/or CNS) by at least about 10%, 20%, 30%, 40%, 50%, 60%, 70%, 80%, 90%, 100%, 200%, 300%, 400%, 500%, 600%, 700%, 800%, 900%, 1000%, or more relative to a reference capsid protein. In some embodiments, capsid proteins increase or enhance transduction of cells or tissues (e.g., liver, muscle, lung, and/or CNS) by at least about 1.2×, 1.5×, 2×, 3×, 4×, 5×, 6×, 7×, 8×, 9×, 10×, 11×, 12×, 13×, 14×, 15×, 16×, 17×, 18×, 19×, 20×, 30×, 40×, 50×, 60×, 70×, 80×, 90×, 100×, or more relative to a reference capsid protein.

### AAV Structure and Function

Adeno-associated virus (AAV) is a parvovirus composed of an icosahedral protein capsid and a single-stranded DNA genome. The AAV viral capsid comprises three subunits, VP1, VP2, and VP3 and two inverted terminal repeat (ITR) regions, which are at the ends of the genomic sequence. The ITRs serve as origins of replication and play a role in viral packaging. The viral genome also comprises rep and cap genes, which are associated with replication and capsid packaging, respectively. In most wild-type AAV, the rep gene encodes four proteins required for viral replication, Rep 78, Rep68, Rep52, and Rep40. The cap gene encodes the capsid subunits as well as the assembly activating protein (AAP), which promotes assembly of viral particles. AAVs are generally replication-deficient, requiring the presence of a helper virus or helper virus functions (e.g., herpes simplex virus (HSV) and/or adenovirus (AdV)) in order to replicate within an infected cell. For example, in some embodiments AAVs require adenoviral E1A, E2A, E4, and VA RNA genes in order to replicate within a host cell.

### Recombinant AAV

In general, recombinant AAV (rAAV) vectors can comprise many of the same elements found in wild-type AAVs, including similar capsid sequences and structures, as well as polynucleotide sequences that are not of AAV origin (e.g., a polynucleotide heterologous to AAV). In some embodiments, rAAVs will replace native, wild-type AAV sequences with polynucleotide sequences encoding a payload. For example, in some embodiments an rAAV will comprise polynucleotide sequences encoding one or more genes intended for therapeutic purposes (e.g., for gene therapy). rAAVs may be modified to remove one or more wild-type viral coding sequences. For example, rAAVs may be engineered to comprise only one ITR, and/or one or more fewer genes necessary for packaging (e.g., rep and cap genes) than would be found in a wild type AAV. Gene expression with rAAVs is generally limited to one or more genes that total 5 kb or less, as larger sequences are not efficiently packaged within the viral capsid. In some embodiments, two or more rAAVs can be used to provide portions of a larger payload, for example, in order to provide an entire coding sequence for a gene that would normally be too large to fit in a single AAV.

Among other things, the present disclosure provides viral vectors comprising one or more polypeptides described herein. In some embodiments, rAAVs may comprise one or more capsid proteins (e.g., one or more capsid proteins from AAVC11.01, AAVC11.02, AAVC11.03, AAVC11.04, AAVC11.05, AAVC11.06, AAVC11.07, AAVC11.08, AAVC11.09, AAVC11.10, AAVC11.11 (referred to interchangeably herein as sL65), AAVC11.12, AAVC11.13, AAVC11.14, AAVC11.15, AAVC11.16, AAVC11.17, AAVC11.18, AAVC11.19, AAV-DJ, and/or AAV-LK03), AAVrh.74, AAVrh.10, AAVhu.37, AAVrh.K, AAVrh.39, AAV12, AAV 13, AAVrh.8, avian AAV, bovine AAV, canine AAV, equine AAV, primate AAV, non-primate AAV, ovine AAV, a hybrid AAV (e.g., an AAV comprising one more sequences of one AAV subtype and one or more sequences of a second subtype), and/or an AAV comprising a mutant AAV capsid protein or a chimeric AAV capsid (e.g., a capsid with polynucleotide sequences derived from two or more different serotypes of AAV). In some embodiments, rAAVs may comprise one or more polynucleotide sequences encoding a gene or nucleic acid of interest (e.g., a gene for treatment of a genetic disease/disorder and/or a inhibitory nucleic acid sequence).

AAV vectors may be capable of being replicated in an infected host cell (replication competent) or incapable of being replicated in an infected host cell (replication incompetent). A replication competent AAV (rcAAV) requires the presence of one or more functional AAV packaging genes. Recombinant AAV vectors are generally designed to be replication-incompetent in mammalian cells, in order to reduce the possibility that rcAAV are generated through recombination with sequences encoding AAV packaging genes. In some embodiments, rAAV vector preparations as described herein are designed to comprise few, if any, rcAAV vectors. In some embodiments, rAAV vector preparations comprise less than about 1 rcAAV per 102 rAAV vectors. In some embodiments, rAAV vector preparations comprise less than about 1 rcAAV per 104 rAAV vectors. In some embodiments, rAAV vector preparations comprise less than about 1 rcAAV per 108 rAAV vectors. In some embodiments, rAAV vector preparations comprise less than about 1 rcAAV per 10¹² rAAV vectors. In some embodiments, rAAV vector preparations comprise no rcAAV vectors.

### Heterologous Nucleic Acids

Among other things, the present disclosure provides polynucleotide cassettes comprising at least one payload and two homology arms, one homology arm at the 5′ end of the polynucleotide cassette and the other homology arm at the 3′ end of the polynucleotide cassette.

### Payloads

In some embodiments, one or more vectors or constructs described herein may comprise a polynucleotide sequence encoding one or more payloads. In accordance with various aspects, any of a variety of payloads may be used (e.g., those with a diagnostic and/or therapeutic purpose), alone or in combination. In some embodiments, a payload may be or comprise a polynucleotide sequence encoding a peptide or polypeptide. In some embodiments, a payload is a peptide that has cell-intrinsic or cell-extrinsic activity that promotes a biological process to treat a medical condition. In some embodiments, a payload may be or comprise a transgene (also referred to herein as a gene of interest (GOI)). In some embodiments, a payload may be or comprise one or more inverted terminal repeat (ITR) sequences (e.g., one or more AAV ITRs). In some embodiments, a payload may be or comprise one or more transgenes with flanking ITR sequences. In some embodiments, a payload may be or comprise one or more heterologous nucleic acid sequences encoding a reporter gene (e.g., a fluorescent or luminescent reporter). In some embodiments, a payload may be or comprise one or more biomarkers (e.g., proxy for payload expression). In some embodiments, a payload may comprise a sequence for polycistronic expression (including, e.g., a 2A peptide, or intronic sequence, internal ribosomal entry site). In some embodiments, 2A peptides are small (e.g., approximately 18-22 amino acids) peptide sequences enabling co-expression of two or more discrete protein products within a single coding sequence. In some embodiments, 2A peptides allows co-expression of two or more discrete protein products regardless of arrangement of protein coding sequences. In some embodiments, 2A peptides are or comprise a consensus motif (e.g., DVEXNPGP). In some embodiments, 2A peptides promote protein cleavage. In some embodiments, 2A peptides are or comprise viral sequences (e.g., foot-and-mouth diseases virus (F2A), equine Rhinitis A virus, porcine teschovirus-1 (P2A), or Thosea asigna virus (T2A)).

In some embodiments, biomarkers are or comprise a 2A peptide (e.g., P2A, T2A, E2A, and/or F2A). In some embodiments, biomarkers are or comprise a Furin cleavage motif (See, Tian et al., FurinDB: A Database of 20-Residue Furin Cleavage Site Motifs, Substrates and Their Associated Drugs, (2011), Int. J. Mol. Sci., vol. 12: 1060-1065). In some embodiments, biomarkers are or comprise a tag (e.g., an immunological tag). In some embodiments, a payload may comprise one or more functional nucleic acids (e.g., one or more siRNA or miRNA). In some embodiments, a payload may comprise one or more inhibitory nucleic acids (including, e.g., ribozyme, miRNA, siRNA, or shRNA, among other things). In some embodiments, a payload may comprise one or more nucleases (e.g., Cas proteins, endonucleases, TALENs, ZFNs).

### Transgenes

In some embodiments, a transgene is a corrective gene chosen to improve one or more signs and/or symptoms of a disease, disorder, or condition. In some embodiments, a transgene may integrate permanently into a host cell genome through use of vector(s) encompassed by the present disclosure. In some embodiments, transgenes are functional versions of disease associated genes (i.e., gene isoform(s) which are associated with the manifestation or worsening of a disease, disorder or condition) found in a host cell. In some embodiments, transgenes are an optimized version of disease-associated genes found in a host cell (e.g., codon optimized or expression-optimized variants). In some embodiments, transgenes are variants of disease-associated genes found in a host cell (e.g., functional gene fragment or variant thereof). In some embodiments, a transgene is a gene that causes expression of a peptide that is normally expressed in one or more healthy tissues. In some embodiments, a transgene is a gene that causes expression of a peptide that is normally expressed in liver cells. In some embodiments, a transgene is a gene that causes expression of a peptide that is normally expressed in muscle cells. In some embodiments, a transgene is a gene that causes expression of a peptide that is normally expressed in central nervous system cells.

In some embodiments, a transgene may be or comprise a gene that causes expression of a peptide that is not normally expressed in one or more healthy tissues (e.g., peptide expressed ectopically). In some embodiments, a transgene is a gene that causes expression of a peptide that is ectopically expressed in one or more healthy tissues (e.g., liver, muscle, central nervous system (CNS), lung). In some embodiments, a transgene is a gene that causes expression of a peptide that is ectopically expressed in one or more healthy tissues and normally expressed in one or more healthy tissues (e.g., liver, muscle, central nervous system (CNS), lung).

In some embodiments, a transgene may be or comprise a gene encoding a functional nucleic acid. In some embodiments, a therapeutic agent is or comprises an agent that has a therapeutic effect upon a host cell or subject (including, e.g., a ribozyme, guide RNA (gRNA), antisense oligonucleotide (ASO), miRNA, siRNA, and/or shRNA). For example, in some embodiments, a therapeutic agent promotes a biological process to treat a medical condition, e.g., at least one symptom of a disease, disorder, or condition.

In some embodiments, transgene expression in a subject results substantially from integration at a target locus. In some embodiments, 75% or more (e.g., 80% or more, 85% or more, 90% or more, 95% or more, 99% or more, 99.5% or more) of total transgene expression in a subject is from transgene integration at a target locus. In some embodiments, 25% or less (e.g., 20% or less, 15% or less, 10% or less, 5% or less, 1% or less, 0.5% or less, 0.1% or less) of total transgene expression in a subject is from a source other than transgene integration at a target locus (e.g., episomal expression, integration at a non-target locus).

In some embodiments, transgenes are transiently expressed in a subject (e.g., episomal expression from plasmids, minicircle DNAs, viruses, etc.). In some embodiments, 75% or more (e.g., 80% or more, 85% or more, 90% or more, 95% or more, 99% or more, 99.5% or more) of total transgene expression in a subject is from transient expression. In some embodiments, 25% or less (e.g., 20% or less, 15% or less, 10% or less, 5% or less, 1% or less, 0.5% or less, 0.1% or less) of total transgene expression in a subject is from a source other than transient expression (e.g., integration at a non-target locus). In some embodiments, transgenes are transiently expressed in a subject (e.g., episomal expression from plasmids, minicircle DNAs, viruses, etc.) for one or more weeks after treatment. In some embodiments, transgenes are transiently expressed in a subject (e.g., episomal expression from plasmids, minicircle DNAs, viruses, etc.) for one or more months after treatment.

In some embodiments, transgenes are transiently expressed in a subject (e.g., episomal expression from plasmids, minicircle DNAs, viruses, etc.) one or more weeks after treatment at a level comparable to that observed within one or more days after treatment. In some embodiments, transgenes are transiently expressed in a subject (e.g., episomal expression from plasmids, minicircle DNAs, viruses, etc.) one or more months after treatment at a level comparable to that observed within one or more days after treatment.

In some embodiments, transgenes are transiently expressed in a subject (e.g., episomal expression from plasmids, minicircle DNAs, viruses, etc.) one or more weeks after treatment at a level that is reduced relative to that observed within one or more days after treatment. In some embodiments, transgenes are transiently expressed in a subject (e.g., episomal expression from plasmids, minicircle DNAs, viruses, etc.) one or more months after treatment at a level that is reduced relative to that observed within one or more days after treatment.

In some embodiments, transgenes are transiently expressed in a subject (e.g., episomal expression from plasmids, minicircle DNAs, viruses, etc.) for no more than one month after treatment. In some embodiments, transgenes are transiently expressed in a subject (e.g., episomal expression from plasmids, minicircle DNAs, viruses, etc.) for no more than two months after treatment. In some embodiments, transgenes are transiently expressed in a subject (e.g., episomal expression from plasmids, minicircle DNAs, viruses, etc.) for no more than three months after treatment. In some embodiments, transgenes are transiently expressed in a subject (e.g., episomal expression from plasmids, minicircle DNAs, viruses, etc.) for no more than four months after treatment. In some embodiments, transgenes are transiently expressed in a subject (e.g., episomal expression from plasmids, minicircle DNAs, viruses, etc.) for no more than five months after treatment. In some embodiments, transgenes are transiently expressed in a subject (e.g., episomal expression from plasmids, minicircle DNAs, viruses, etc.) for no more than six months after treatment.

In some embodiments, combined size of transgenes and homology arms can be optimized to increase the likelihood that these transgenes are of a suitable sequence length to be efficiently packaged in a delivery vehicle, which can increase the likelihood that the transgenes will ultimately be delivered appropriately in the patient.

In some embodiments, a nucleotide sequence encoding a transgene is codon-optimized. In some embodiments, a nucleotide sequence encoding a transgene is codon-optimized for a certain cell type (e.g., mammalian, insect, bacterial, fungal, etc.). In some embodiments, a nucleotide sequence encoding a transgene is codon-optimized for a human cell. In some embodiments, a nucleotide sequence encoding a transgene is codon-optimized for a human cell of a particular tissue type (e.g., liver, muscle, CNS, lung).

In certain embodiments, a nucleotide sequence encoding a transgene may be codon optimized to have a nucleotide homology with a reference nucleotide sequence (e.g., a wild-type gene sequence) of less than 100%. In certain embodiments, nucleotide homology between a codon-optimized nucleotide sequence encoding a transgene and a reference nucleotide sequence is less than 100%, less than 99%, less than 98%, less than 97%, less than 96%, less than 95%, less than 94%, less than 93%, less than 92%, less than 91%, less than 90%, less than 89%, less than 88%, less than 87%, less than 86%, less than 85%, less than 84%, less than 83%, less than 82%, less than 81%, less than 80%, less than 78%, less than 76%, less than 74%, less than 72%, less than 70%, less than 68%, less than 66%, less than 64%, less than 62%, less than 60%, less than 55%, less than 50%, and less than 40%.

In some embodiments, a transgene may be or comprise a sequence having at least 80%, 85%, 90%, 95%, 99%, or 100% identity to a corresponding wild-type reference nucleotide sequence (e.g., a wild-type gene sequence). In some embodiments, a transgene may be or comprise a sequence having a least 80%, 85%, 90%, 95%, 99%, or 100% identity to a portion of a corresponding wild-type reference nucleotide sequence (e.g., a wild-type gene sequence). In some embodiments, a transgene may be or comprise a sequence having at least 80%, 85%, 90%, 95%, 99%, or 100% identity to an exemplary sequence in Table 5 below.

TABLE 5
Exemplary transgene sequences

| Transgene | | |
|---|---|---|
| Description | Sequence | SEQ ID NO |
| Human FAH | tccttcatcccggtggccgaggattccgacttccccatcc | 4 |
| | acaacctgccctacggcgtcttctcgaccagaggcgaccc | |
| | aagaccgaggataggtgtggccattggcgaccagatcctg | |
| | gacctcagcatcatcaagcacctctttactggtcctgtcc | |
| | tctccaaacaccaggatgtcttcaatcagcctacactcaa | |
| | cagcttcatgggcctgggtcaggctgcctggaaggaggcg | |
| | agagtgttcttgcagaacttgctgtctgtgagccaagcca | |
| | ggctcagagatgacaccgaacttcggaagtgtgcattcat | |
| | ctcccaggcttctgccacgatgcaccttccagccaccata | |
| | ggagactacacagacttctattcctctcggcagcatgcta | |
| | ccaacgtcggaatcatgttcagggacaaggagaatgcgtt | |
| | gatgccaaattggctgcacttaccagtgggctaccatggc | |
| | cgtgcctcctctgtcgtggtgtctggcaccccaatccgaa | |
| | ggcccatgggacagatgaaacctgatgactctaagcctcc | |
| | cgtatatggtgcctgcaagctcttggacatggagctggaa | |
| | atggctttttttgtaggccctggaaacagattgggagagc | |
| | cgatccccatttccaaggcccatgagcacatttttggaat | |
| | ggtccttatgaacgactggagtgcacgagacattcagaag | |
| | tgggagtatgtccctctcgggccattccttgggaagagtt | |
| | ttgggaccactgtctctccgtgggtggtgcccatggatgc | |
| | tctcatgccctttgctgtgcccaacccgaagcaggacccc | |
| | aggcccctgccgtatctgtgccatgacgagccctacacat | |
| | ttgacatcaacctctctgttaacctgaaaggagaaggaat | |
| | gagccaggcggctaccatatgcaagtccaattttaagtac | |
| | atgtactggacgatgctgcagcagctcactcaccactctg | |
| | tcaacggctgcaacctgcggccgggggacctcctggcttc | |
| | tgggaccatcagcgggccggagccagaaaacttcggctcc | |
| | atgttggaactgtcgtggaagggaacgaagcccatagacc | |
| | tggggaatggtcagaccaggaagtttctgctggacgggga | |
| | tgaagtcatcataacagggtactgccagggggatggttac | |
| | cgcatcggctttggccagtgtgctggaaaagtgctgcctg | |
| | ctctcctgccatca | |
| Human FAH | tccttcattcctgtggccgaggattctgactttcccattc | 5 |
| Codon | acaacctgccctatggcgtcttttcaaccaggggagatcc | |
| optimized | caggcccagaatcggggggccattggagatcagatcctgg | |
| variant 3 | acctgtcaatcatcaagcacctgtttacaggccctgtgct | |
| | gagcaagcaccaggatgtgttcaaccagccaactctgaac | |
| | agcttcatggggctggggcaggctgcctggaaggaggcaa | |
| | gggtgtttctgcagaatctgctgagcgtgtcacaggctag | |
| | gctgagggatgataccgagctgaggaagtgtgcatttatc | |
| | tcacaggcatcagccacaatgcatctgccagctacaatcg | |
| | gcgactataccgacttttactcaagcaggcagcatgccac | |
| | caatgtgggcatcatgttcagggacaaagagaatgccctg | |
| | atgccaaattggctgcacctgcctgtgggctatcatggca | |
| | gagccagctcagtggtggtgtcagggacaccaattaggag | |
| | gcctatgggccagatgaagcctgatgacagcaaaccacct | |
| | gtctacggcgcctgcaagctcctggatatggaactggaga | |
| | tggctttttttgtgggcccaggcaatagactgggagagcc | |
| | cattccaatctcaaaggcccatgaacatatctttggcatg | |
| | gtcctgatgaatgactggtcagccagagacattcagaagt | |
| | gggagtacgtgccactgggaccatttctgggaaagagctt | |
| | tggcaccacagtgtcaccatgggtggtgcccatggatgcc | |
| | ctgatgccctttgctgtgccaaatccaaaacaagacccca | |
| | ggcccctcccctatctctgtcatgatgaaccatatacttt | |
| | tgacattaacctgagcgtgaacctgaaaggagaaggcatg | |
| | agccaagccgccactatctgtaagagcaacttcaaatata | |
| | tgtactggacaatgctgcagcagctcacccaccatagcgt | |
| | gaatgggtgtaacctgaggccaggcgacctgctggcatca | |
| | ggcactatttcagggcctgagcctgaaaattttggatcaa | |
| | tgctggagctgtcatggaagggaactaaacccatcgacct | |
| | ggggaatggccagaccagaaagtttctcctggatggcgat | |
| | gaggtgatcattacaggctactgtcagggggatggctata | |
| | gaattggatttggccaatgcgccggcaaagtcctccctgc | |
| | cctgctgcccagc | |
| Human FAH | tccttcatcccagtggctgaagattctgacttccccattc | 6 |
| Codon | acaacctcccttatggagtctttagcacaagaggagaccc | |
| optimized | aagacctagaattggcgtggctattggggatcagatcctg | |
| variant 2 | gacctgagcattatcaaacatctctttacaggcccagtcc | |
| | tcagcaagcaccaagatgtgttcaatcaacccacactgaa | |
| | ctcatttatggggctgggccaagctgcctggaaggaggca | |
| | agagtgtttctccagaacctcctgtcagtgagccaggcca | |
| | ggctgagagatgacacagagctgaggaagtgtgcctttat | |
| | ctcacaagccagcgccacaatgcacctgccagcaaccatc | |
| | ggggactacacagacttttacagcagcaggcagcacgcca | |
| | ctaatgtgggcatcatgtttagagacaaagagaatgccct | |
| | catgcctaactggctgcatctgccagtgggctaccatggc | |
| | agagccagcagcgtggtggtgtctggcacccctatcagga | |
| | ggcccatgggccagatgaagcctgatgacagcaagccacc | |
| | tgtgtatggggcatgtaagctgctggacatggagctggaa | |
| | atggctttctttgtggggcctgggaacaggctgggcgaac | |
| | caattcccatcagcaaagctcatgaacacatttttgggat | |
| | ggtgctcatgaatgactggtctgccagagacatccagaag | |
| | tgggagtatgtccccctgggaccattcctgggcaagagct | |
| | ttgggaccactgtgtcaccatgggtggtgcccatggatgc | |
| | cctgatgccatttgctgtgcctaatcccaaacaagatcca | |
| | aggccactgccctacctctgccacgatgagccctacacat | |
| | ttgatatcaacctctctgtgaatctgaagggagaaggaat | |
| | gagccaagctgctaccatttgcaaaagcaactttaagtat | |
| | atgtattggaccatgctgcagcaactcacccaccacagcg | |
| | tgaatggctgtaacctgaggcctggcgacctgctggcctc | |
| | tggcaccatcagcggccctgaacctgagaactttggcagc | |
| | atgctggagctgagctggaagggaaccaagcccattgacc | |
| | tgggcaatggacagaccaggaaattcctcctggacgggga | |
| | cgaggtgatcatcacaggctattgccagggggatgggtat | |
| | aggattggctttggccaatgtgccggcaaagtgctgcctg | |
| | ccctgctccctagc | |
| Human FAH | tcctttattcctgtggccgaagatagcgacttccctatcc | 7 |
| Codon | ataacctcccatatggcgtcttctcaaccaggggcgaccc | |
| optimized | caggcccaggattggggggcaattggagaccagatcctgg | |
| variant 1 | acctcagcatcattaagcacctctttacaggacctgtgct | |
| | gagcaagcaccaggatgtgttcaaccagcctaccctgaat | |
| | agctttatgggactgggccaggcagcatggaaagaagcca | |
| | gagtgttcctccagaatctgctgagcgtgagccaggccag | |
| | gctcagggatgacacagaactgaggaaatgtgccttcatc | |
| | tcacaggcatcagccacaatgcacctccctgccacaattg | |
| | gggactacactgacttctacagcagcagacagcatgccac | |
| | taatgtggggattatgtttagagacaaggaaaatgccctc | |
| | atgccaaattggctgcacctgcctgtgggctaccacggca | |
| | gagcctcatcagtggtggtgtcaggcacaccaattaggag | |
| | gccaatgggacagatgaagcctgacgactcaaagccacca | |
| | gtctatggcgcctgcaaactgctggacatggagctggaga | |
| | tggctttctttgtggggcctggcaacaggctgggagaacc | |
| | aatccctatttcaaaggcccacgagcacatttttggcatg | |
| | gtgctcatgaatgactggtctgccagagatatccagaagt | |
| | gggaatacgtgcccctgggcccttttctgggcaagagctt | |
| | tggcaccacagtgtcaccttgggtggtcccaatggatgcc | |
| | ctgatgccctttgccgtgcccaaccccaagcaggatccca | |
| | gaccactgccctacctgtgccacgatgagccctatacctt | |
| | tgacatcaacctgtcagtcaacctgaagggggagggcatg | |
| | agccaggccgccactatttgcaagagcaactttaaatata | |
| | tgtactggactatgctccaacagctcactcaccactcagt | |
| | gaatggctgcaatctgaggcctggcgacctcctggctagc | |
| | ggcactatctctggccctgaacctgagaactttgggagca | |
| | tgctggagctgtcatggaaagggacaaagcctattgacct | |
| | ggggaatggccagacaaggaagtttctcctggatggcgat | |
| | gaggtcatcatcactgggtactgccagggcgatggctaca | |
| | ggattggatttggacagtgtgctggcaaagtgctcccagc | |
| | tctgctgccctca | |
| Human | CCTGAGCAGGAGAGACAGATCACAGCCAGAGAAGGGGCCA | 8 |
| truncated | GTCGGAAAATCTTATCTAAGCTTTCTTTGCCTACCCGTGC | |
| ATP7B | CTGGGAACCAGCAATGAAGAAGAGTTTTGCTTTTGACAAT | |
| | GTTGGCTATGAAGGTGGTCTGGATGGCCTGGGCCCTTCTT | |
| | CTCAGCCGCAGAAGTGCTTCTTACAGATCAAAGGCATGAC | |
| | CTGTGCATCCTGTGTGTCTAACATAGAAAGGAATCTGCAG | |
| | AAAGAAGCTGGTGTTCTCTCCGTGTTGGTTGCCTTGATGG | |
| | CAGGAAAGGCAGAGATCAAGTATGACCCAGAGGTCATCCA | |
| | GCCCCTCGAGATAGCTCAGTTCATCCAGGACCTGGGTTTT | |
| | GAGGCAGCAGTCATGGAGGACTACGCAGGCTCCGATGGCA | |
| | ACATTGAGCTGACAATCACAGGGATGACCTGCGCGTCCTG | |
| | TGTCCACAACATAGAGTCCAAACTCACGAGGACAAATGGC | |
| | ATCACTTATGCCTCCGTTGCCCTTGCCACCAGCAAAGCCC | |
| | TTGTTAAGTTTGACCCGGAAATTATCGGTCCACGGGATAT | |
| | TATCAAAATTATTGAGGAAATTGGCTTTCATGCTTCCCTG | |
| | GCCCAGAGAAACCCCAACGCTCATCACTTGGACCACAAGA | |
| | TGGAAATAAAGCAGTGGAAGAAGTCTTTCCTGTGCAGCCT | |
| | GGTGTTTGGCATCCCTGTCATGGCCTTAATGATCTATATG | |
| | CTGATACCCAGCAACGAGCCCCACCAGTCCATGGTCCTGG | |
| | ACCACAACATCATTCCAGGACTGTCCATTCTAAATCTCAT | |
| | CTTCTTTATCTTGTGTACCTTTGTCCAGCTCCTCGGTGGG | |
| | TGGTACTTCTACGTTCAGGCCTACAAATCTCTGAGACACA | |
| | GGTCAGCCAACATGGACGTGCTCATCGTCCTGGCCACAAG | |
| | CATTGCTTATGTTTATTCTCTGGTCATCCTGGTGGTTGCT | |
| | GTGGCTGAGAAGGCGGAGAGGAGCCCTGTGACATTCTTCG | |
| | ACACGCCCCCCATGCTCTTTGTGTTCATTGCCCTGGGCCG | |
| | GTGGCTGGAACACTTGGCAAAGAGCAAAACCTCAGAAGCC | |
| | CTGGCTAAACTCATGTCTCTCCAAGCCACAGAAGCCACCG | |
| | TTGTGACCCTTGGTGAGGACAATTTAATCATCAGGGAGGA | |
| | GCAAGTCCCCATGGAGCTGGTGCAGCGGGGCGATATCGTC | |
| | AAGGTGGTCCCTGGGGGAAAGTTTCCAGTGGATGGGAAAG | |
| | TCCTGGAAGGCAATACCATGGCTGATGAGTCCCTCATCAC | |
| | AGGAGAAGCCATGCCAGTCACTAAGAAACCCGGAAGCACT | |
| | GTAATTGCGGGGTCTATAAATGCACATGGCTCTGTGCTCA | |
| | TTAAAGCTACCCACGTGGGCAATGACACCACTTTGGCTCA | |
| | GATTGTGAAACTGGTGGAAGAGGCTCAGATGTCAAAGGCA | |
| | CCCATTCAGCAGCTGGCTGACCGGTTTAGTGGATATTTTG | |
| | TCCCATTTATCATCATCATGTCAACTTTGACGTTGGTGGT | |
| | ATGGATTGTAATCGGTTTTATCGATTTTGGTGTTGTTCAG | |
| | AGATACTTTCCTAACCCCAACAAGCACATCTCCCAGACAG | |
| | AGGTGATCATCCGGTTTGCTTTCCAGACGTCCATCACGGT | |
| | GCTGTGCATTGCCTGCCCCTGCTCCCTGGGGCTGGCCACG | |
| | CCCACGGCTGTCATGGTGGGCACCGGGGTGGCCGCGCAGA | |
| | ACGGCATCCTCATCAAGGGAGGCAAGCCCCTGGAGATGGC | |
| | GCACAAGATAAAGACTGTGATGTTTGACAAGACTGGCACC | |
| | ATTACCCATGGCGTCCCCAGGGTCATGCGGGTGCTCCTGC | |
| | TGGGGGATGTGGCCACACTGCCCCTCAGGAAGGTTCTGGC | |
| | TGTGGTGGGGACTGCGGAGGCCAGCAGTGAACACCCCTTG | |
| | GGCGTGGCAGTCACCAAATACTGTAAAGAGGAACTTGGAA | |
| | CAGAGACCTTGGGATACTGCACGGACTTCCAGGCAGTGCC | |
| | AGGCTGTGGAATTGGGTGCAAAGTCAGCAACGTGGAAGGC | |
| | ATCCTGGCCCACAGTGAGCGCCCTTTGAGTGCACCGGCCA | |
| | GTCACCTGAATGAGGCTGGCAGCCTTCCCGCAGAAAAAGA | |
| | TGCAGTCCCCCAGACCTTCTCTGTGCTGATTGGAAACCGT | |
| | GAGTGGCTGAGGCGCAACGGTTTAACCATTTCTAGCGATG | |
| | TCAGTGACGCTATGACAGACCACGAGATGAAAGGACAGAC | |
| | AGCCATCCTGGTGGCTATTGACGGTGTGCTCTGTGGGATG | |
| | ATCGCAATCGCAGACGCTGTCAAGCAGGAGGCTGCCCTGG | |
| | CTGTGCACACGCTGCAGAGCATGGGTGTGGACGTGGTTCT | |
| | GATCACGGGGGACAACCGGAAGACAGCCAGAGCTATTGCC | |
| | ACCCAGGTTGGCATCAACAAAGTCTTTGCAGAGGTGCTGC | |
| | CTTCGCACAAGGTGGCCAAGGTCCAGGAGCTCCAGAATAA | |
| | AGGGAAGAAAGTCGCCATGGTGGGGGATGGGGTCAATGAC | |
| | TCCCCGGCCTTGGCCCAGGCAGACATGGGTGTGGCCATTG | |
| | GCACCGGCACGGATGTGGCCATCGAGGCAGCCGACGTCGT | |
| | CCTTATCAGAAATGATTTGCTGGATGTGGTGGCTAGCATT | |
| | CACCTTTCCAAGAGGACTGTCCGAAGGATACGCATCAACC | |
| | TGGTCCTGGCACTGATTTATAACCTGGTTGGGATACCCAT | |
| | TGCAGCAGGTGTCTTCATGCCCATCGGCATTGTGCTGCAG | |
| | CCCTGGATGGGCTCAGCGGCCATGGCAGCCTCCTCTGTGT | |
| | CTGTGGTGCTCTCATCCCTGCAGCTCAAGTGCTATAAGAA | |
| | GCCTGACCTGGAGAGGTATGAGGCACAGGCGCATGGCCAC | |
| | ATGAAGCCCCTGACGGCATCCCAGGTCAGTGTGCACATAG | |
| | GCATGGATGACAGGTGGCGGGACTCCCCCAGGGCCACACC | |
| | ATGGGACCAGGTCAGCTATGTCAGCCAGGTGTCGCTGTCC | |
| | TCCCTGACGTCCGACAAGCCATCTCGGCACAGCGCTGCAG | |
| | CAGACGATGATGGGGACAAGTGGTCTCTGCTCCTGAATGG | |
| | CAGGGATGAGGAGCAGTACATC | |

### Homology Arms

In some embodiments, viral vectors described herein comprise one or more flanking polynucleotide sequences with significant sequence homology to a target locus (e.g., homology arms). In some embodiments, homology arms flank a polynucleotide sequence encoding a payload (e.g., one homology arm is 5′ to a payload (also referred to herein as a 5′ homology arm) and one homology arm is 3′ to a payload (also referred to herein as a 3′ homology arm)). In some embodiments, homology arms direct site-specific integration of a payload.

In some embodiments, homology arms are of the same length (also referred to herein as balanced homology arms or even homology arms). In some embodiments, viral vectors comprising homology arms of the same length, wherein the homology arms are at least a certain length, provide improved effects (e.g., improved rate of target integration). In some embodiments, homology arms are between 100 nt and 1000 nt in length. In some embodiments, homology arms are between 200 nt and 1000 nt in length. In some embodiments, homology arms are between 500 nt and 1500 nt in length. In some embodiments, homology arms are between 1000 nt and 2000 nt in length. In some embodiments, homology arms are greater than 2000 nt in length. In some embodiments, each homology arm is at least 750 nt in length. In some embodiments, each homology arm is at least 1000 nt in length. In some embodiments, each homology arm is at least 1250 nt in length. In some embodiments, homology arms are less than 1000 nt in length. In some embodiments, homology arms contain at least 70% homology to a target locus. In some embodiments, homology arms contain at least 80% homology to a target locus. In some embodiments, homology arms contain at least 90% homology to a target locus. In some embodiments, homology arms contain at least 95% homology to a target locus. In some embodiments, homology arms contain at least 99% homology to a target locus. In some embodiments, homology arms contain 100% homology to a target locus.

In some embodiments, homology arms are of different lengths (also referred to herein as unbalanced homology arms or uneven homology arms). In some embodiments, viral vectors comprising unbalanced homology arms of different lengths provide improved effects (e.g., increased rate of target site integration) as compared to a reference sequence. In some embodiments, viral vectors comprising homology arms of different lengths, wherein each homology arm is at least a certain length, provide improved effects (e.g., increased rate of target site integration) as compared to a reference sequence (e.g., a viral vector comprising homology arms of the same length or a viral vector comprising one or more homology arms less than 1000 nt in length).

In some embodiments, each homology arm is greater than 500 nt in length. In some embodiments, each homology arm is at least 750 nt length. In some embodiments, each homology arm is at least 1000 nt in length. In some embodiments, one homology arm is at least 750 nt in length and another homology arm is at least 1000 nt in length. In some embodiments, one homology arm is at least 750 nt in length and another homology arm is at least 1100 nt in length. In some embodiments, one homology arm is at least 750 nt in length and another homology arm is at least 1200 nt in length. In some embodiments, one homology arm is at least 750 nt in length and another homology arm is at least 1300 nt in length. In some embodiments, one homology arm is at least 750 nt in length and another homology arm is at least 1400 nt in length. In some embodiments, one homology arm is at least 750 nt in length and another homology arm is at least 1500 nt in length. In some embodiments, one homology arm is at least 750 nt in length and another homology arm is at least 1600 nt in length. In some embodiments, one homology arm is at least 750 nt in length and another homology arm is at least 1700 nt in length. In some embodiments, one homology arm is at least 750 nt in length and another homology arm is at least 1800 nt in length. In some embodiments, one homology arm is at least 750 nt in length and another homology arm is at least 1900 nt in length. In some embodiments, one homology arm is at least 750 nt in length and another homology arm is at least 2000 nt in length. In some embodiments, one homology arm is at least 1000 nt in length and another homology arm is at least 1100 nt in length. In some embodiments, one homology arm is at least 1000 nt in length and another homology arm is at least 1200 nt in length. In some embodiments, one homology arm is at least 1000 nt in length and another homology arm is at least 1300 nt in length. In some embodiments, one homology arm is at least 1000 nt in length and another homology arm is at least 1400 nt in length. In some embodiments, one homology arm is at least 1000 nt in length and another homology arm is at least 1500 nt in length. In some embodiments, one homology arm is at least 1000 nt in length and another homology arm is at least 1600 nt in length. In some embodiments, one homology arm is at least 1000 nt in length and another homology arm is at least 1700 nt in length. In some embodiments, one homology arm is at least 1000 nt in length and another homology arm is at least 1800 nt in length. In some embodiments, one homology arm is at least 1000 nt in length and another homology arm is at least 1900 nt in length. In some embodiments, one homology arm is at least 1000 nt in length and another homology arm is at least 2000 nt in length. In some embodiments, one homology arm is at least 1300 nt in length and another homology arm is at least 1400 nt in length In some embodiments, a 5′ homology arm is longer than a 3′ homology arm. In some embodiments, a 3′ homology arm is longer than a 5′ homology arm.

In some embodiments, viral vectors comprising homology arms provide improved effects (e.g., increased rate of target site integration) as compared to a reference sequence (e.g., viral vectors lacking homology arms). In some embodiments, viral vectors comprising homology arms provide rates of target site integration of 0.01% or more (e.g., 0.05% or more, 0.1% or more, 0.2% or more, 0.3% or more, 0.4% or more, 0.5% or more, 0.6% or more, 0.7% or more, 0.8% or more, 0.9% or more, 1% or more, 1.5% or more, 2% or more, 5% or more, 10% or more, 20% or more, 30% or more). In some embodiments, viral vectors comprising homology arms provide increasing rates of target site integration over time. In some embodiments, rates of target site integration increase over time relative to an initial measurement of target site integration. In some embodiments, rates of target site integration over time are at least 1.5× higher than an initial measurement of target site integration (e.g., 1.5×, 2×, 3×, 4×, 5×, 10×, 20×, 30×, 40×, 50×, 60×, 70×, 80×, 90×, 100×, 200×). In some embodiments, rates of target site integration are measured after one or more days. In some embodiments, rates of target site integration are measured after one or more weeks. In some embodiments, rates of target site integration are measured after one or more months. In some embodiments, rates of target site integration are measured after one or more years.

In some embodiments, viral vectors comprising homology arms of different lengths provide improved effects (e.g., increased rate of target site integration) relative to a reference sequence (e.g., viral vectors with homology arms of the same length, viral vectors with at least one homology arm below 500 nt). In some embodiments, viral vectors comprising homology arms of different lengths provide at least 1.1×, at least 1.2×, at least 1.3×, at least 1.4×, at least 1.5×, at least 1.6×, at least 1.7×, at least 1.8×, at least 1.9×, at least 2.0×, at least 2.5×, at least 3.0×, at least 3.5×, or at least 4.0× improved editing activity relative to a reference composition (e.g., viral vectors with homology arms of the same length, viral vectors with at least one homology arm below 500 nt).

In some embodiments, viral vectors comprising homology arms of different lengths provide rates of target site integration of 0.01% or more (e.g., 0.05% or more, 0.1% or more, 0.2% or more, 0.3% or more, 0.4% or more, 0.5% or more, 0.6% or more, 0.7% or more, 0.8% or more, 0.9% or more, 1% or more, 1.5% or more, 2% or more, 5% or more, 10% or more, 20% or more, 30% or more). In some embodiments, viral vectors comprising homology arms of different lengths provide increasing rates of target site integration over time. In some embodiments, rates of target site integration increase over time relative to an initial measurement of target site integration. In some embodiments, rates of target site integration over time are at least 1.5× higher than an initial measurement of target site integration (e.g., 1.5×, 2×, 3×, 4×, 5×, 10×, 20×, 30×, 40×, 50×, 60×, 70×, 80×, 90×, 100×, 200×). In some embodiments, rates of target site integration are measured after one or more days. In some embodiments, rates of target site integration are measured after one or more weeks. In some embodiments, rates of target site integration are measured after one or more months. In some embodiments, rates of target site integration are measured after one or more years. In some embodiments, rates of target site integration are measured through assessment of one or more biomarkers (e.g., biomarkers comprising a 2A peptide). In some embodiments, rates of target site integration are measured through assessment of one or more isolated nucleic acids (e.g., mRNA, gDNA). In some embodiments, rates of target site integration are measured through assessment of gene expression (e.g., through immunohistochemical staining).

TABLE 1
Exemplary methods for assessment of target site integration

| | | | |
|---|---|---|---|
| Genomic DNA | Liver | qPCR | Liver biopsy subjected to genomic |
| integration rate | (frozen) | | DNA extraction. qPCR method run to |
| (gDNA Int %) | | | detect percentage of allele (e.g. |
| | | | albumin) containing on-target |
| | | | insertion. |
| Fused mRNA | Liver | ddPCR | Liver biopsy subjected to RNA |
| | (frozen) | | extraction. ddPCR method run to |
| | | | quantify the copy number of fused |
| | | | mRNA (unique chimeric mRNA |
| | | | transcribed from edited allele). This |
| | | | assay measures the transcriptional |
| | | | activity after target insertion. |
| ALB-2A | Plasma | ELISA | Blood collected and processed for |
| | | | plasma. Proprietary ELISA used to |
| | | | measure 2A-tagged albumin |
| | | | (universal circulating biomarker for |
| | | | targeted integration) This assy |
| | | | measures total protein expression |
| | | | after target insertion. |
| Hepatocyte | Fixed | IHC | Fixed liver sectioned and stained |
| editing % | liver | | against transgene. Transgene-positive |
| | section | | cells counted and used to calculate |
| | | | percentage of hepatocyte editing. For |
| | | | targeted integration into the albumin |
| | | | locus, transgene expression should be |
| | | | hepatocyte-specific. This assay |
| | | | focuses on per-cell target integration |
| | | | and is orthogonal to gDNA Int %, |
| | | | which focuses on per allele target |
| | | | integration. |

In some embodiments, viral vectors comprising homology arms of different lengths may provide improved gene editing in a species or a model system for a species (e.g., mouse, human, or models thereof). In some embodiments, viral vectors may comprise different combinations of homology arm lengths when optimized for expression in a particular species or a model system for a particular species (e.g., mouse, human, or models thereof). In some embodiments, viral vectors comprising specific combinations of homology arm lengths may provide improved gene editing in one species or a model system of one species (e.g., human, humanized mouse model) as compared to a second species or a model system of a second species (e.g., mouse, pure mouse model). In some embodiments, viral vectors comprising specific combinations of homology arm lengths may be optimized for high levels of gene editing in one species or a model of one species (e.g., human, humanized mouse model) as compared to a second species or a model system of a second species (e.g., mouse, pure mouse model).

In some embodiments, homology arms direct integration of a transgene immediately behind a highly expressed endogenous gene. In some embodiments, homology arms direct integration of a transgene without disrupting endogenous gene expression (non-disruptive integration).

### Homology Arm Design and Optimization

In some embodiments, viral vectors may comprise different combinations of homology arm lengths when optimized for expression.

The present disclosure encompasses the recognition that different designs of homology arms may be advantageous in the context of various viral vectors (e.g., viral vectors comprising different expression cassettes including a transgene and 2A sequence). For example, in some embodiments, if a viral vector comprises an expression cassette comprising a transgene, a 2A sequence (e.g., P2A), and ITRs that total a combined length of at least 2.7 kb (e.g., leaving approximately 2 kb or less for homology arms given the packaging capacity of a typical AAV), then use of homology arms of the same length (i.e., a balanced design) may be preferred to maintain a length as close to 1 kb length as possible for each homology arm. Such a design may provide improved integration efficiency.

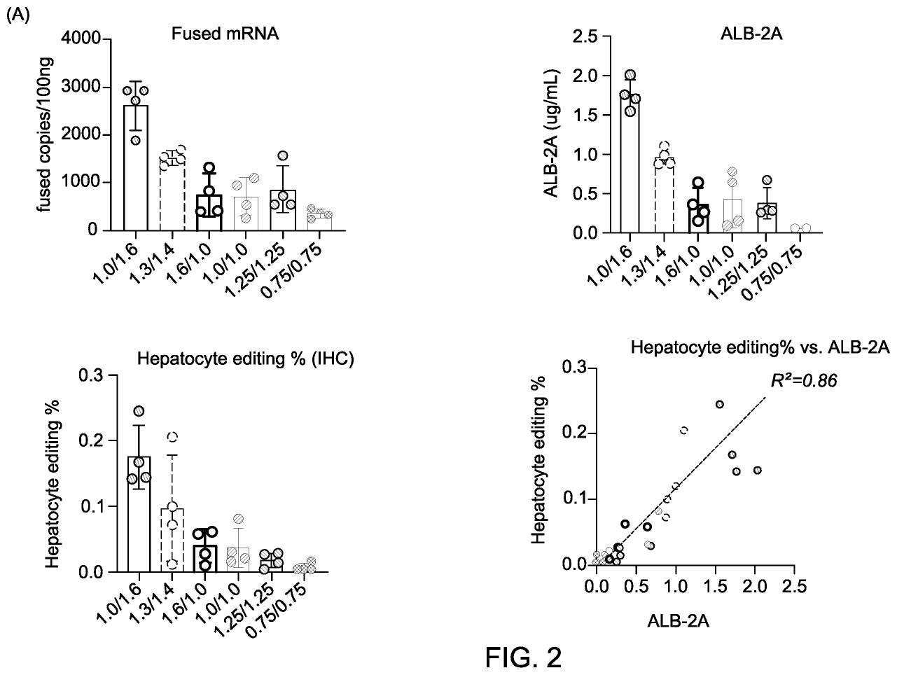

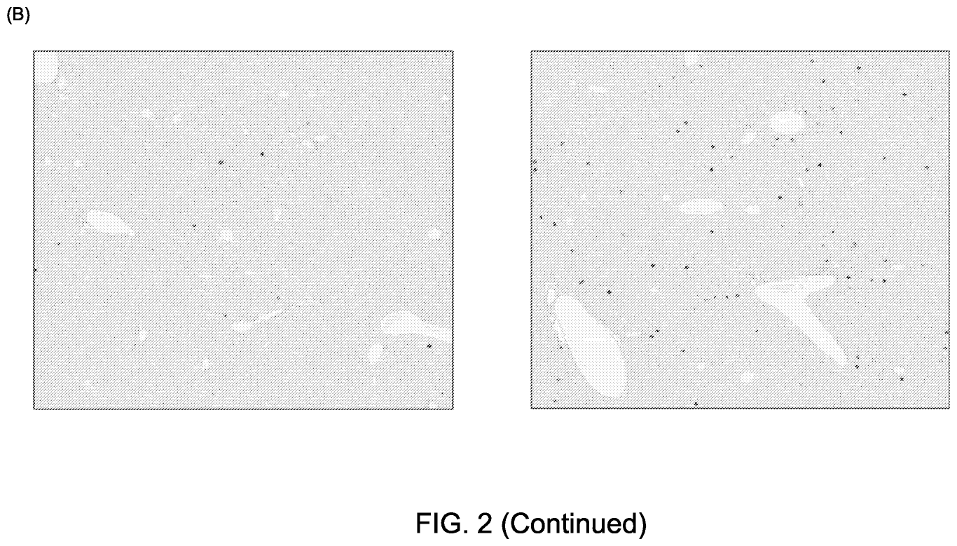

In some other embodiments, as demonstrated in FIG. 2, if a viral vector comprises an expression cassette comprising a transgene, a 2A sequence (e.g., P2A), and ITRs that total less than 2.7 kb (e.g., leaving more than 2 kb for homology arms given the packaging capacity of a typical AAV), then use of homology arms of different lengths (i.e., an unbalanced design) is preferred and may provide improved integration efficiency. In some embodiments, a viral vector may comprise homology arms of different lengths with a 3′ homology arm of 1 kb, and a longer 5′ homology arm (e.g., the max allowed by the packaging capacity of the vector. It is contemplated that such a design may provide improved integration efficiency in a species (e.g., humans)

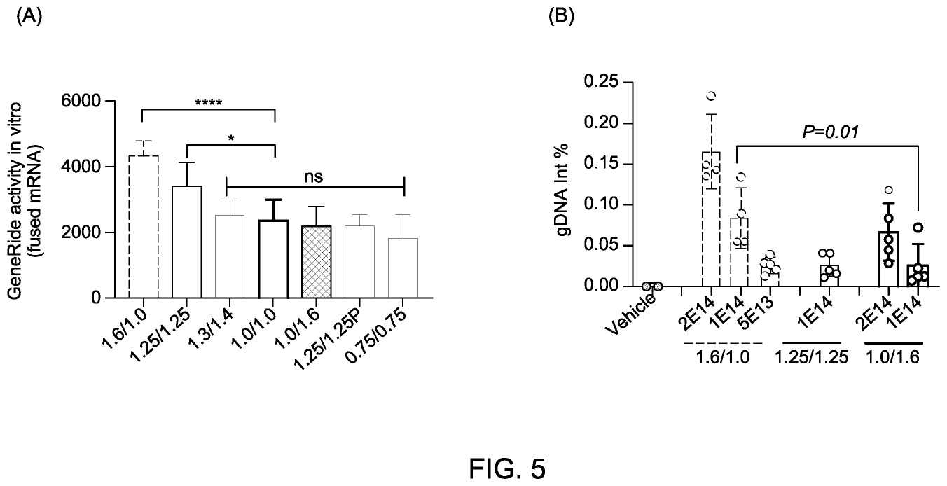

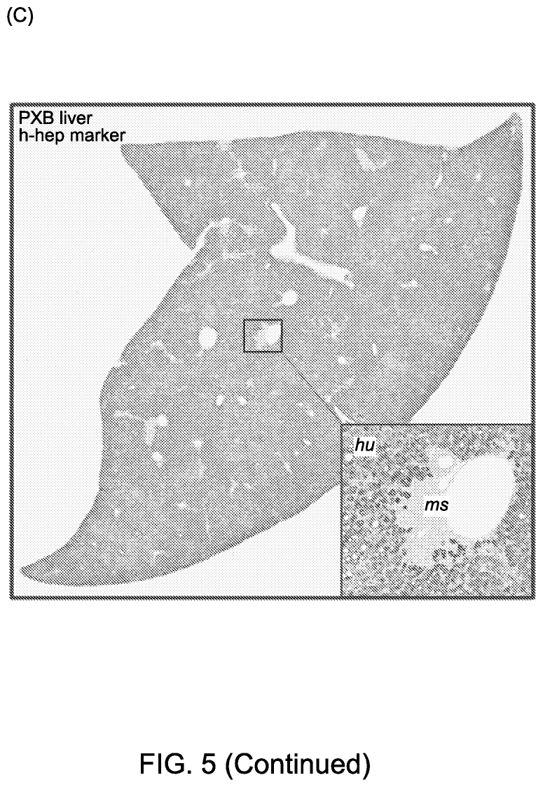

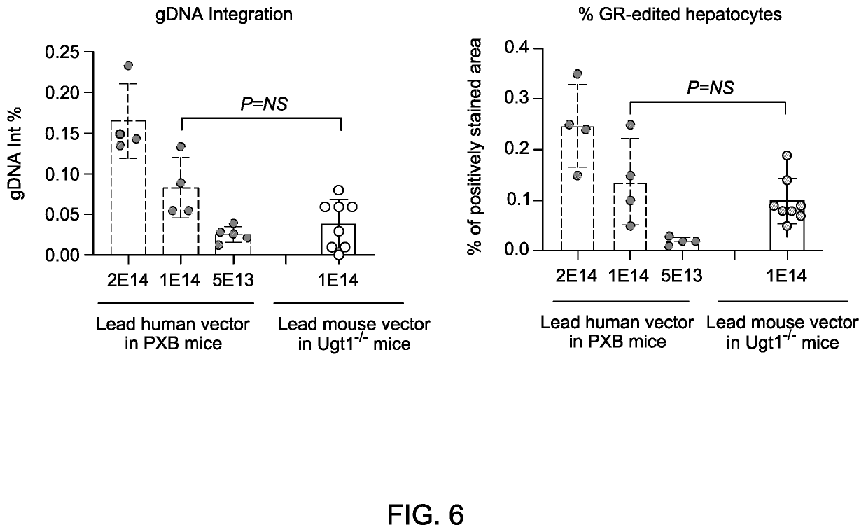

The present disclosure encompasses the recognition that different designs of homology arms may be advantageous in the context of gene editing in a species or a model system for a species (e.g., mouse, human, or models thereof). For example, in some embodiments, as demonstrated in FIG. 5, if a viral vector comprises an expression cassette comprising a transgene, a 2A sequence (e.g., P2A), and ITRs that totals less than 2.7 kb (e.g., leaving more than 2 kb for homology arms given the packaging capacity of a typical AAV), then use of homology arms of different lengths (i.e., an unbalanced design) with a 3′ homology arm of 1 kb, and a longer 5′ homology arm (e.g., the max allowed by the packaging capacity of the vector (e.g., 1.6 kb)) is preferred and may provide improved integration efficiency in a species (e.g., humans). In contrast, in some embodiments, as demonstrated in FIG. 2, if viral vectors as described herein, comprise an expression cassette comprising a transgene, a 2A sequence (e.g., P2A), and ITRs that total less than 2.7 kb (e.g., leaving more than 2 kb for homology arms given the packaging capacity of a typical AAV), then use of homology arms of different lengths (i.e., an unbalanced design) with a 5′ homology arm of 1 kb and a longer 3′ homology arm (e.g., the max allowed by the packaging capacity of the vector (e.g., 1.6 kb)) is preferred and may provide improved integration efficiency in a species (e.g., mice).

### Methods of Treatment

Compositions and constructs disclosed herein may be used in any in vitro or in vivo application wherein expression of a payload (e.g. transgene) from a particular target locus in a cell, while maintaining expression of endogenous genes at and surrounding the target locus, is desired. For example, compositions and constructs disclosed herein may be used to treat a disorder, disease, or medical condition in a subject (e.g., through gene therapy).

In some embodiments, treatment comprises obtaining or maintaining a desired pharmacologic and/or physiologic effect. In some embodiments, a desired pharmacologic and/or physiologic effect may comprise completely or partially preventing a disease (e.g., preventing symptoms of disease). In some embodiments, a desired pharmacologic and/or physiologic effect may comprise completely or partially curing a disease (e.g., curing adverse effects associated with a disease). In some embodiments, a desired pharmacologic and/or physiologic effect may comprise preventing recurrence of a disease. In some embodiments, a desired pharmacologic and/or physiologic effect may comprise slowing progression of a disease. In some embodiments, a desired pharmacologic and/or physiologic effect may comprise relieving symptoms of a disease. In some embodiments, a desired pharmacologic and/or physiologic effect may comprise preventing regression of a disease. In some embodiments, a desired pharmacologic and/or physiologic effect may comprise stabilizing and/or reducing symptoms associated with a disease.

In some embodiments, treatment comprises administering a composition before, during, or after onset of a disease (e.g., before, during, or after appearance of symptoms associated with a disease). In some embodiments, treatment comprises combination therapy (e.g., with one or more therapies, including different types of therapies).

### Diseases of Interest

In some embodiments, compositions and constructs disclosed herein may be used to treat any disease of interest that includes a genetic deficiency or abnormality as a component of the disease.

By way of specific example, in some embodiments, compositions and constructs such as those disclosed herein may be used to treat branched-chain organic acidurias (e.g., Maple Syrup Urine Disease (MSUD), methylmalonic acidemia (MMA), propionic acidemia (PA), isovaleric acidemia (IVA), argininosuccinic aciduria). In some embodiments, treatment comprises introduction of a polynucleotide sequence encoding one or more transgenes of interest (e.g., BCKDH complex (E1a, E1b, and E2 subunits), methylmalonyl-CoA mutase, propionyl-CoA carboxylase (alpha and beta subunits), isovaleryl CoA dehydrogenase, argininosuccinate lyase (ASL), and/or variants thereof). In some embodiments, treatment comprises reduction of aberrant proteins (e.g., non-functional proteins) associated with branched chain organic acidurias. In some embodiments, treatment comprises reduction of signs and/or symptoms associated with branched chain organic acidurias (e.g., hypotonia, developmental delay, seizures, optic atrophy, acute encephalopathy, hyperventilation, respiratory distress, temperature instability, recurrent vomiting, ketoacidosis, pancreatitis, constipation, neutropenia, pancytopenia, secondary hemophagocytosis, cardiac arrhythmia, cardiomyopathy, chronic renal failure, dermatitis, hearing loss).

In some embodiments, compositions and constructs disclosed herein may be used to treat fatty acid oxidation disorders (e.g., trifunctional protein deficiency, Long-chain L-3 hydroxyacyl-CoA dehydrogenase (LCAD) deficiency, Medium-chain acyl-CoA dehydrogenase (MCHAD) deficiency, Very long-chain acyl-CoA dehydrogenase (VLCHAD) deficiency). In some embodiments, treatment comprises introduction of a polynucleotide sequence encoding one or more transgenes of interest (e.g., HADHA, HADHB, LCHAD, ACADM, ACADVL, and/or variants thereof). In some embodiments, treatment comprises reduction of aberrant proteins (e.g., non-functional proteins) associated with fatty acid oxidation disorders. In some embodiments, treatment comprises reduction of signs and/or symptoms associated with fatty acid oxidation disorders (e.g., enlarged liver, delayed mental and physical development, cardiac muscle weakness, cardiac arrhythmia, nerve damage, abnormal liver function, rhabdomyolysis, myoglobinuria, hypoglycemia, metabolic acidosis, respiratory distress, hepatomegaly, hypotonia, cardiomyopathy).

In some embodiments, compositions and constructs disclosed herein may be used to treat glycogen storage diseases (e.g., glycogen storage disease type 1 (GSD1), glycogen storage disease type 2 (Pompe disease, GSD2). In some embodiments, treatment comprises introduction of a polynucleotide sequence encoding one or more transgenes of interest (e.g., G6PC (GSD1a), G6PT1 (GSD1b), SLC17A3, SLC37A4 (GSD1c), acid alpha-glucosidase, and/or variants thereof). In some embodiments, treatment comprises reduction of aberrant proteins (e.g., non-functional proteins) associated with glycogen storage diseases. In some embodiments, treatment comprises reduction of signs and/or symptoms associated with glycogen storage diseases (e.g., enlarged liver, hypoglycemia, muscle weakness, muscle cramps, fatigue, delayed development, obesity, bleeding disorders, abnormal liver function, abnormal kidney function, abnormal respiratory function, abnormal cardiac function, mouth sores, gout, cirrhosis, fibrosis, liver tumors).

In some embodiments, compositions and constructs disclosed herein may be used to treat carnitine cycle disorders. In some embodiments, treatment comprises introduction of a polynucleotide sequence encoding one or more transgenes of interest (e.g., OCTN2, CPT1, CACT, CPT2, and/or variants thereof). In some embodiments, treatment comprises reduction of aberrant proteins (e.g., non-functional proteins) associated with carnitine cycle disorders. In some embodiments, treatment comprises reduction of signs and/or symptoms associated with carnitine cycle disorders (e.g., hypoketotic hypoglycemia, cardiomyopathy, muscle weakness, fatigue, delayed motor development, edema).

In some embodiments, compositions and constructs disclosed herein may be used to treat urea cycle disorders. In some embodiments, treatment comprises introduction of a polynucleotide sequence encoding one or more transgenes of interest (e.g., CPS1, ARG1, ASL, OTC, and/or variants thereof). In some embodiments, treatment comprises reduction of aberrant proteins (e.g., non-functional proteins) associated with urea cycle disorders. In some embodiments, treatment comprises reduction of signs and/or symptoms associated with urea cycle disorders (e.g., vomiting, nausea, behavior abnormalities, fatigue, coma, psychosis, lethargy, cyclical vomiting, myopia, hyperammonemia, elevated ornithine levels).

In some embodiments, compositions and constructs disclosed herein may be used to treat Crigler-Najjar syndrome. In some embodiments, treatment comprises introduction of a polynucleotide sequence encoding one or more transgenes of interest (e.g., UGT1A1, and/or variants thereof). In some embodiments, treatment comprises reduction of aberrant proteins (e.g., non-functional proteins) associated with Crigler-Najjar syndrome. In some embodiments, treatment comprises reduction of signs and/or symptoms associated with Crigler-Najjar syndrome (e.g., jaundice, kemicterus, lethargy, vomiting, fever, abnormal reflexes, muscle spasms, opisthotonus, spasticity, hypotonia, athetosis, elevated bilirubin levels, diarrhea, slurred speech, confusion, difficulty swallowing, seizures).

In some embodiments, compositions and constructs disclosed herein may be used to treat hereditary tyrosinemia. In some embodiments, treatment comprises introduction of a polynucleotide sequence encoding one or more transgenes of interest (e.g., FAH, and/or variants thereof). In some embodiments, treatment comprises reduction of aberrant proteins (e.g., non-functional proteins) associated with hereditary tyrosinemia. In some embodiments, treatment comprises reduction of signs and/or symptoms associated with hereditary tyrosinemia (e.g., hepatomegaly, jaundice, liver disease, cirrhosis, hepatocarcinoma, fever, diarrhea, melena, vomiting, splenomegaly, edema, coagulopathy, abnormal kidney function, rickets, weakness, hypertonia, ileus, tachycardia, hypertension, neurological crises, respiratory failure, cardiomyopathy).

In some embodiments, compositions and constructs disclosed herein may be used to treat epidermolysis bullosa. In some embodiments, treatment comprises introduction of a polynucleotide sequence encoding one or more transgenes of interest (e.g., COL7A1, COL17A1, MMP1, KRT5, LAMA3, LAMB3, LAMC2, ITGB4, and/or variants thereof). In some embodiments, treatment comprises reduction of aberrant proteins (e.g., non-functional proteins) associated with epidermolysis bullosa. In some embodiments, treatment comprises reduction of signs and/or symptoms associated with epidermolysis bullosa (e.g., fragile skin, abnormal nail growth, blisters, thickened skin, scarring alopecia, atrophic scarring, milia, dental problems, dysphagia, skin itching and pain).

In some embodiments, compositions and constructs disclosed herein may be used to treat alpha-1 antitrypsin deficiency (A1ATD). In some embodiments, treatment comprises introduction of a polynucleotide sequence encoding one or more transgenes of interest (e.g., alpha-1 antitrypsin (A1AT), and/or variants thereof). In some embodiments, treatment comprises reduction of aberrant proteins (e.g., non-functional proteins) associated with alpha-1 antitrypsin deficiency. In some embodiments, treatment comprises reduction of signs and/or symptoms associated with A1ATD (e.g., emphysema, chronic cough, phlegm production, wheezing, chronic respiratory infections, jaundice, enlarged liver, bleeding, abnormal fluid accumulation, elevated liver enzymes, liver dysfunction, portal hypertension, fatigue, edema, chronic active hepatitis, cirrhosis, hepatocarcinoma, panniculitis).

In some embodiments, compositions and constructs disclosed herein may be used to treat Wilson's disease. In some embodiments, treatment comprises introduction of a polynucleotide sequence encoding one or more transgenes of interest (e.g., ATP7B, and/or variants thereof). In some embodiments, treatment comprises reduction of aberrant proteins (e.g., non-functional proteins) associated with Wilson's disease. In some embodiments, treatment comprises reduction of signs and/or symptoms associated with Wilson's disease (e.g., fatigue, lack of appetite, abdominal pain, jaundice, Kayser-Fleischer rings, edema, speech problems, problems swallowing, loss of physical coordination, uncontrolled movements, muscle stiffness, liver disease, anemia, depression, schizophrenia, ammenorrhea, infertility, kidney stones, renal tubular damage, arthritis, osteoporosis, osteophytes)

In some embodiments, compositions and constructs disclosed herein may be used to treat hematologic diseases (e.g., hemophilia A, hemophilia B). In some embodiments, treatment comprises introduction of a polynucleotide sequence encoding one or more transgenes of interest (e.g., Factor IX (FIX), Factor VIII (FVIII), and/or variants thereof). In some embodiments, treatment comprises reduction of aberrant proteins (e.g., non-functional proteins) associated with hematologic diseases. In some embodiments, treatment comprises reduction of signs and/or symptoms associated with hematologic diseases (e.g., excessive bleeding, abnormal bruising, joint pain and swelling, bloody urine, bloody stool, abnormal nosebleeds, headache, lethargy, vomiting, double vision, weakness, convulsions, seizures).

In some embodiments, compositions and constructs disclosed herein may be used to treat hereditary angioedema. In some embodiments, treatment comprises introduction of a polynucleotide sequence encoding one or more transgenes of interest (e.g., C1 esterase inhibitor (C1-inh)). In some embodiments, treatment comprises reduction of aberrant proteins (e.g., non-functional proteins) associated with hereditary angioedema. In some embodiments, treatment comprises reduction of signs and/or symptoms associated with hereditary angioedema (e.g., edema, pruritus, urticaria, nausea, vomiting, acute abdominal pain, dysphagia, dysphonia, stridor).

In some embodiments, compositions and constructs disclosed herein may be used to treat Parkinson's disease. In some embodiments, treatment comprises introduction of a polynucleotide sequence encoding one or more transgenes of interest (e.g., dopamine decarboxylase (DDC)). In some embodiments, treatment comprises reduction of aberrant proteins (e.g., non-functional proteins) associated with Parkinson's disease. In some embodiments, treatment comprises reduction of signs and/or symptoms associated with Parkinson's disease (e.g., tremors, bradykinesia, muscle stiffness, impaired posture and balance, loss of automatic movements, speech changes, writing changes).

In some embodiments, compositions and constructs disclosed herein may be used to treat muscular diseases. In some embodiments, treatment comprises introduction of a polynucleotide sequence encoding one or more transgenes of interest (e.g., muscular dystrophies, Duchenne's muscular dystrophy (DMD), limb girdle muscular dystrophy). X-linked myotubular myopathy). In some embodiments, treatment comprises reduction of aberrant proteins (e.g., non-functional proteins) associated with muscular diseases. In some embodiments, treatment comprises reduction of signs and/or symptoms associated with muscular diseases (e.g., difficult movement, enlarged calf muscles, muscle pain and stiffness, delayed development, learning disabilities, unusual gait, scoliosis, breathing problems, difficulty swallowing, arrhythmia, cardiomyopathy, abnormal joint function, hypotonia, respiratory distress, absence of reflexes).

In some embodiments, compositions and constructs disclosed herein may be used to treat mucopolysaccharidosis (MPS) (e.g., MPS IH, MPS IH/S, MPS IS, MPS II, MPS IIIA, MPS IIIB, MPS IIIC, MPS HID, MPS IVA, MPS IVB, MPS V, MPS VI, MPS VII, MPS IX). In some embodiments, treatment comprises introduction of a polynucleotide sequence encoding one or more transgenes of interest (e.g., IDUA, IDS, SGSH, NAGLU, HGSNAT, GNS, GALNS, GLB1, ARSB, GUSB, HYAL1). In some embodiments, treatment comprises reduction of aberrant proteins (e.g., non-functional proteins) associated with mucopolysaccharidosis. In some embodiments, treatment comprises reduction of signs and/or symptoms associated with MPS (e.g., heart abnormalities, breathing irregularities, enlarged liver, enlarged spleen, neurological abnormalities, developmental delays, recurring infections, persistent nasal discharge, noisy breathing, clouding of the cornea, enlarged tongue, spine deformities, joint stiffness, carpal tunnel, aortic regurgitation, progressive hearing loss, seizures, unsteady gait, accumulation of heparan sulfate, enzyme deficiencies, abnormal skeleton and musculature, heart disease, cysts, soft-tissue masses).

In some embodiments, compositions and constructs disclosed herein may be used to treat lysosomal acid lipase deficiency. In some embodiments, treatment comprises introduction of a polynucleotide sequence encoding one or more transgenes of interest (e.g., LIPA and/or variants thereof). In some embodiments, treatment comprises reduction of aberrant proteins (e.g., non-functional proteins) associated with lysosomal acid lipase deficiency. In some embodiments, treatment comprises reductions of signs and/or symptoms associated with lysosomal acid lipase deficiency (e.g., vomiting, diarrhea, swelling of the abdomen, and failure to gain weight, weight loss, jaundice, fever, calcification, anemia, liver dysfunction or failure, cachexia, malabsorption, bile duct problems, cardiac disease, stroke).

In some embodiments, compositions and constructs disclosed herein may be used to treat homocystinuria (HCU). In some embodiments, treatment comprises introduction of a polynucleotide sequence encoding one or more transgenes of interest (e.g., cystathionine-beta-synthase, or CBS gene, MTHFR, and/or variants thereof). In some embodiments, treatment comprises an increase in the ability of a subject to metabolize methionine. In some embodiments, treatment comprises reductions of signs and/or symptoms associated with HCU (e.g., nearsightedness, intellectual impairment, in ability to gain weight, weak bones, seizures, blood clots).

In some embodiments, compositions and constructs disclosed herein may be used to treat disorders associated with bile acid metabolism, transport, and/or cholestasis. In some embodiments, treatment comprises introduction of a polynucleotide sequence encoding one or more transgene of interest (e.g., PFIC1, PFIC2, PFIC3, ABCB4, and/or variant thereof). In some embodiments, treatment comprises reduction of aberrant proteins (e.g., non-functional proteins) associated with bile acid metabolism, transport, and/or cholestasis. In some embodiments, treatment comprises reductions of signs and/or symptoms associated with bile acid metabolism, transport, and/or cholestasis (e.g., itching, jaundice, failure to thrive, portal hypertension, hepatosplenomegaly, diarrhea, pancreatitis, hepatocellular carcinoma).

In some embodiments, compositions and constructs disclosed herein may be used to treat phenylketonuria. In some embodiments, treatment comprises introduction of a polynucleotide sequence encoding one or more transgene of interest (e.g., phenylalanine hydroxylase (PAH) and/or variant thereof). In some embodiments, treatment comprises reduction of aberrant proteins (e.g., non-functional proteins) associated with phenylketonuria. In some embodiments, treatment comprises reductions of signs and/or symptoms associated with phenylketonuria (e.g., musty odor in the breath, skin and/or urine, seizures, skin rashes, microcephaly, hyperactivity, intellectual disability, asthma, eczema, anemia, weight gain, renal insufficiency, osteoporosis, gastritis, esophagus, and kidney deficiencies, kidney stones, hypertension, psychiatric problems, dizziness).

In some embodiments, compositions and constructs disclosed herein may be used to treat primary hyperoxaluria. In some embodiments, treatment comprises introduction of a polynucleotide sequence encoding one or more transgene of interest (e.g., AGT, AGXT, GRHPR, HOGA1, and/or variant thereof). In some embodiments, treatment comprises reduction of aberrant proteins (e.g., non-functional proteins) associated with primary hyperoxaluria. In some embodiments, treatment comprises reductions of signs and/or symptoms associated with phenylketonuria (e.g., flank pain, oxalosis, kidney stones and/or stones elsewhere in the urinary tract such as the bladder or urethra, nephrocalcinosis, hematuria, dysuria, the urge to urinate often, renal colic, blockage of the urinary tract, repeated urinary tract infections, kidney damage, kidney failure, failure to thrive).

In some embodiments, compositions and constructs disclosed herein may be used to treat porphyrias. In some embodiments, treatment comprises introduction of a polynucleotide sequence encoding one or more transgene of interest (e.g., ALAD, HMBS, UROS, UROD, CPOX, PPOCX, FECH, ALAS2, and/or variant thereof). In some embodiments, treatment comprises reduction of aberrant proteins (e.g., non-functional proteins) associated with porphyrias. In some embodiments, treatment comprises reductions of signs and/or symptoms associated with porphyrias (e.g., abdominal pain, pain in the arms and leg, generalized weakness, vomiting, confusion, constipation, tachycardia, fluctuating blood pressure, urinary retention, psychosis, hallucinations, seizures, abrasions, blisters, erosions of the skin, skin lesions, nausea, increased blood pressure, confusion).

In some embodiments, compositions and constructs disclosed herein may be used to treat disorders associated with production of antibodies (e.g., autoimmune disorders). In some embodiments, treatment comprises introduction of a polynucleotide sequence encoding one or more transgenes of interest (e.g., POLB, HLA-DRB1, IL7R, CYP27B1, TNFRSF1A, HLA-B, HLA-DPB1, HLA-DRB1, IRF5, PTPN22, RBPJ, RUNX1, STAT4, and/or variants thereof). In some embodiments, treatment comprises reduction of aberrant proteins (e.g., non-functional proteins) associated with production of antibodies. In some embodiments, treatment comprises reduction of signs and/or symptoms associated with production of antibodies (e.g., swollen joints, joint stiffness, fatigue, fever, appetite loss, vision problems, tremor, unsteady gait, dizziness, skin rash, lesions, hyperalgesia).

In some embodiments, compositions and constructs disclosed herein may be used to treat disorders associated with production of secreted proteins. In some embodiments, treatment comprises introduction of a polynucleotide sequence encoding one or more transgenes of interest. In some embodiments, treatment comprises reduction of aberrant proteins (e.g., non-functional proteins) associated with production of secreted proteins. In some embodiments, treatment comprises reduction of signs and/or symptoms associated with production of secreted proteins.

### Targeted Integration

In some embodiments, compositions and constructs provided herein direct integration of a payload (e.g., a transgene and/or functional nucleic acid) at a target locus (e.g., an endogenous gene). In some embodiments, compositions and constructs provided herein direct integration of a payload at a target locus in a specific cell type (e.g., tissue-specific loci). In some embodiments, integration of a payload occurs in a specific tissue (e.g., liver, central nervous system (CNS), muscle, kidney, vascular. lung). In some embodiments, integration of a payload occurs in multiple tissues (e.g., liver, central nervous system (CNS), muscle, kidney, vascular, lung).

In some embodiments, compositions and constructs provided herein direct integration of a payload at a target locus that is considered a safe-harbor site (e.g., albumin, Apolipoprotein A2 (ApoA2), Haptoglobin). In some embodiments, a target locus may be selected from any genomic site appropriate for use with methods and compositions provided herein. In some embodiments, a target locus encodes a polypeptide. In some embodiments, a target locus encodes a polypeptide that is highly expressed in a subject (e.g., a subject not suffering from a disease, disorder, or condition, or a subject suffering from a disease, disorder, or condition). In some embodiments, integration of a payload occurs at a 5′ or 3′ end of one or more endogenous genes (e.g., genes encoding polypeptides). In some embodiments, integration of a payload occurs between a 5′ or 3′ end of one or more endogenous genes (e.g., genes encoding polypeptides).

In some embodiments, compositions and constructs provided herein direct integration of a payload at a target locus with minimal or no off-target integration (e.g., integration at a non-target locus). In some embodiments, compositions and constructs provided herein direct integration of a payload at a target locus with reduced off-target integration compared to a reference composition or construct (e.g., relative to a composition or construct without flanking homology sequences).

In some embodiments, integration of a transgene at a target locus allows expression of a payload without disrupting endogenous gene expression. In some embodiments, integration of a transgene at a target locus allows expression of a payload from an endogenous promoter. In some embodiments, integration of a transgene at a target locus disrupts endogenous gene expression. In some embodiments, integration of a transgene at a target locus disrupts endogenous gene expression without adversely affecting a target cell and/or subject (e.g., by targeting a safe-harbor site). In some embodiments, integration of a transgene at a target locus does not require use of a nuclease (e.g., Cas proteins, endonucleases, TALENs, ZFNs). In some embodiments, integration of a transgene at a target locus is assisted by use of a nuclease (e.g., Cas proteins, endonucleases, TALENs, ZFNs).

In some embodiments, integration of a transgene at a target locus confers a selective advantage (e.g., increased survival rate in a plurality of cells relative to other cells in a tissue). In some embodiments, a selective advantage may produce an increased percentage of cells in one or more tissues expressing a transgene.

### Compositions

In some embodiments, compositions can be produced using methods and constructs provided herein (e.g., viral vectors). In some embodiments, compositions include liquid, solid, and gaseous compositions. In some embodiments, compositions comprise additional ingredients (e.g., diluents, stabilizer, excipients, adjuvants). In some embodiments, additional ingredients can comprise buffers (e.g., phosphate, citrate, organic acid buffers), antioxidants (e.g., ascorbic acid), low molecular weight polypeptides (e.g., less than 10 residues), various proteins (e.g., serum albumin, gelatin, immunoglobulins), hydrophilic polymers (e.g., polyvinylpyrrolidone), amino acids (e.g., glycine, glutamine, asparagine, arginine, lysine), carbohydrates (e.g., monosaccharides, disaccharides, glucose, mannose, dextrins), chelating agents (e.g., EDTA), sugar alcohols (e.g., mannitol, sorbitol), salt-forming counterions (e.g., sodium, potassium), and/or nonionic surfactants (e.g. Tween™, Pluronics™, polyethylene glycol (PEG)), among other things. In some embodiments, an aqueous carrier is an aqueous pH buffered solution.

In some embodiments, compositions provided herein may be provided in a range of dosages. In some embodiments, compositions provided herein may be provided in a single dose. In some embodiments, compositions provided herein may be provided in multiple dosages. In some embodiments, compositions are provided over a period of time. In some embodiments, compositions are provided at specific intervals (e.g., varying intervals, set intervals). In some embodiments, dosages may vary depending upon dosage form and route of administration. In some embodiments, compositions provided herein may be provided in dosages between 1e11 and 1e14 vg/kg. In some embodiments, compositions provided herein may be provided in dosages between 1e11 and 1e12 vg/kg. In some embodiments, compositions provided herein may be provided in dosages between 1e12 and 1e13 vg/kg. In some embodiments, compositions provided herein may be provided in dosages between 1e12 and 1e14 vg/kg. In some embodiments, compositions provided herein may be provided in dosages between 1e14 and 1e15 vg/kg. In some embodiments, compositions provided herein may be provided in dosages of no more than 1e14 vg/kg. In some embodiments, compositions provided herein may be provided in dosages of no more than 1e15 vg/kg.

In some embodiments, compositions provided herein may be administered to a subject at a particular timepoint (e.g., age of a subject). In some embodiments, compositions provided herein may be administered to a newborn subject. In some embodiments, compositions provided herein may be administered to a neonatal subject. In some embodiments, a neonatal mouse subject is between 0 and 7 days of age. In some embodiments, a neonatal human subject is between 0 days and 1 month of age. In some embodiments compositions provided herein may be administered to a subject between 7 days of age and 30 days of age. In some embodiments, compositions provided herein may be administered to a subject between 3 months of age and 1 year of age. In some embodiments, compositions provided herein may be administered to a subject between 1 year of age and 5 years of age. In some embodiments, compositions provided herein may be administered to a subject between 4 years of age and 7 years of age. In some embodiments, compositions provided herein may be administered to a subject at 5 years of age or older.

In some embodiments, compositions provided herein may be administered to a subject at a particular timepoint based upon growth stage (e.g., percentage of estimated/average adult size or weight) of a particular tissue or organ. In some embodiments, compositions provided herein may be administered to a subject wherein a tissue or organ (e.g., liver, muscle, CNS, lung, etc.) is at least 10%, at least 20%, at least 30%, at least 40%, at least 50%, at least 60%, at least 70%, at least 80%, at least 90%, or at least 99% of estimated/average adult size or weight. In some embodiments, compositions provided herein may be administered to a subject wherein a tissue or organ is approximately 20% (+/−5%) of estimated/average adult size or weight. In some embodiments, compositions provided herein may be administered to a subject wherein a tissue or organ is approximately 50% (+/−5%) of estimated/average adult size or weight. In some embodiments, compositions provided herein may be administered to a subject wherein a tissue or organ is approximately 60% (+/−5%) of estimated/average adult size or weight. In some embodiments, estimated/average adult size or weight of a particular tissue or organ may be determined as described in the art (See, Noda et al. Pediatric radiology, 1997; Johnson et al. Liver transplantation, 2005; and Szpinda et al. Biomed research international, 2015, each of which is incorporated herein by reference in its entirety.

### Routes of Administration

In some embodiments, compositions provided herein may be administered to a subject via any one (or more) of a variety of routes known in the art (e.g., parenteral, subcutaneous, intravenous, intracranial, intraspinal, intraocular, intramuscular, intravaginal, intraperitoneal, epicutaneous, intradermal, rectal, pulmonary, intraosseous, oral, buccal, intraportal, intra-arterial, intratracheal, or nasal). In some embodiments, compositions provided herein may be introduced into cells, which are then introduced into a subject (e.g., liver, muscle, central nervous system (CNS), lung, hematologic cells). In some embodiments, compositions provided herein may be introduced via delivery methods known in the art (e.g., injection, catheter).

### Methods of Producing Viral Vectors

### Production of Viral Vectors

In some embodiments, production of viral vectors (e.g., AAV viral vectors) may include both upstream steps to generate viral vectors (e.g. cell-based culturing) and downstream steps to process viral vectors (e.g., purification, formulation, etc.). In some embodiments, upstream steps may comprise one or more of cell expansion, cell culture, cell transfection, cell lysis, viral vector production, and/or viral vector harvest.

In some embodiments, downstream steps may comprise one or more of separation, filtration, concentration, clarification, purification, chromatography (e.g., affinity, ion exchange, hydrophobic, mixed-mode), centrifugation (e.g., ultracentrifugation), and/or formulation.

In some embodiments, constructs and methods described herein are designed to increase viral vector yields (e.g., AAV vector yields), reduce levels of replication-competent viral vectors (e.g., replication competent AAV (rcAAV)), improve viral vectors packaging efficiency (e.g., AAV vector capsid packaging), and/or any combinations thereof, relative to a reference construct or method, for example those in Xiao et al. 1998 and Grieger et al. 2015, each of which is incorporated herein by reference in its entirety.

### Cell Lines and Transfection Reagents

In some embodiments, production of viral vectors comprises use of cells (e.g., cell culture). In some embodiments, production of viral vectors comprises use cell culture of one or more cell lines (e.g., mammalian cell lines). In some embodiments, production of viral vectors comprises use of HEK293 cell lines or variants thereof (e.g., HEK293T, HEK293F cell lines). In some embodiments, cells are capable of being grown in suspension. In some embodiments, cells are comprised of adherent cells. In some embodiments, cells are capable of being grown in media that does not comprise animal components (e.g. animal serum). In some embodiments, cells are capable of being grown in serum-free media (e.g., F17 media, Expi293 media). In some embodiments, production of viral vectors comprises transfection of cells with expression constructs (e.g., plasmids). In some embodiments, cells are selected for high expression of viral vectors (e.g. AAV vectors). In some embodiments, cells are selected for high packaging efficiency of viral vectors (e.g., capsid packaging of AAV vectors). In some embodiments, cells are selected for improved transfection efficiency (e.g., with chemical transfection reagents, including cationic molecules). In some embodiments, cells are engineered for high expression of viral vectors (e.g. AAV vectors). In some embodiments, cells are engineered for high packaging efficiency of viral vectors (e.g., capsid packaging of AAV vectors). In some embodiments, cells are engineered for improved transfection efficiency (e.g., with chemical transfection reagents, including cationic molecules). In some embodiments, cells may be engineered or selected for two or more of the above attributes. In some embodiments, cells are contacted with one or more expression constructs (e.g. plasmids). In some embodiments, cells are contacted with one or more transfection reagents (e.g., chemical transfection reagents, including lipids, polymers, and cationic molecules) and one or more expression constructs. In some embodiments, cells are contacted with one or more cationic molecules (e.g., cationic lipid, PEI reagent) and one or more expression constructs. In some embodiments, cells are contacted with a PEIMAX reagent and one or more expression constructs. In some embodiments, cells are contacted with a FectoVir-AAV reagent and one or more expression constructs. In some embodiments, cells are contacted with one or more transfection reagents and one or more expression constructs at particular ratios. In some embodiments, ratios of transfection reagents to expression constructs improves production of viral vectors (e.g., improved vector yield, improved packaging efficiency, and/or improved transfection efficiency).

### Expression Constructs

In some embodiments, expression constructs are or comprise one or more polynucleotide sequences (e.g., plasmids). In some embodiments, expression constructs comprise particular polynucleotide sequence elements (e.g., payloads, promoters, viral genes, etc.). In some embodiments, expression constructs comprise polynucleotide sequences encoding viral genes (e.g., a rep or cap gene or gene variant, one or more helper virus genes or gene variants). In some embodiments, expression constructs of a particular type comprise specific combinations of polynucleotide sequence elements. In some embodiments, expression constructs of a particular type do not comprise specific combinations of polynucleotide sequence elements. In some embodiments, a particular expression construct does not comprise polynucleotide sequence elements encoding both rep and cap genes and/or gene variants.

In some embodiments, expression constructs comprise polynucleotide sequences encoding wild-type viral genes (e.g., wild-type rep genes, cap genes, viral helper genes, or combinations thereof). In some embodiments, expression constructs comprise polynucleotide sequences encoding viral helper genes or gene variants (e.g., herpesvirus genes or gene variants, adenovirus genes or gene variants). In some embodiments, expression constructs comprise polynucleotide sequences encoding one or more gene copies that express one or more wild-type Rep proteins (e.g., 1 copy, 2 copies, 3 copies, 4 copies, 5 copies, etc.). In some embodiments, expression constructs comprise polynucleotide sequences encoding a single gene copy that expresses one or more wild-type Rep proteins (e.g., Rep68, Rep40, Rep52, Rep78, or combinations thereof). In some embodiments, expression constructs comprise polynucleotide sequences encoding one or more wild-type Rep proteins (e.g., Rep68, Rep40, Rep52, Rep78, or combinations thereof). In some embodiments, expression constructs comprise polynucleotide sequences encoding at least four wild-type Rep proteins (e.g., Rep68, Rep40, Rep52, Rep78). In some embodiments, expression constructs comprise polynucleotide sequences encoding each of Rep68, Rep40, Rep52, and Rep78. In some embodiments, expression constructs comprise polynucleotide sequences encoding one or more wild-type adenoviral helper proteins (e.g., E2 and E4).

In some embodiments, expression constructs comprise wild-type polynucleotide sequences encoding wild-type viral genes (e.g., rep genes, cap genes, helper genes). In some embodiments, expression constructs comprise modified polynucleotide sequences (e.g., codon-optimized) encoding wild-type viral genes (e.g., rep genes, cap genes, helper genes). In some embodiments, expression constructs comprise modified polynucleotide sequences encoding modified viral genes (e.g., rep genes, cap genes, helper genes). In some embodiments, modified viral genes are designed and/or engineered for certain improvements (e.g., improved transduction, tissue specificity, reduced size, reduced immune response, improved packaging, reduced rcAAV levels, etc.).

In accordance with various embodiments, expression constructs disclosed herein may offer increased flexibility and modularity as compared to previous technologies. In some embodiments, expression constructs disclosed herein may allow swapping of various polynucleotide sequences (e.g., different rep genes, cap genes, payloads, helper genes, promoters, etc.) while providing certain improvements (e.g., increased viral vector yield, increased packaging, reduced rcAAV levels, etc.). In some embodiments, expression constructs disclosed herein are compatible with various upstream production processes (e.g., different cell culture conditions, different transfection reagents, etc.) while providing certain improvements (e.g., increased viral vector yield, increased packaging, reduced rcAAV levels, etc.)

In some embodiments, expression constructs of different types comprise different combinations of polynucleotide sequences. In some embodiments, an expression construct of one type comprises one or more polynucleotide sequence elements (e.g., payloads, promoters, viral genes, etc.) that is not present in an expression construct of a different type. In some embodiments, an expression construct of one type comprises polynucleotide sequence elements encoding a viral gene (e.g., a rep or cap gene or gene variant) and polynucleotide sequence elements encoding a payload (e.g., a transgene and/or functional nucleic acid). In some embodiments, an expression construct of one type comprises polynucleotide sequence elements encoding one or more viral genes (e.g., a rep or cap gene or gene variant and/or one or more helper virus genes). In some embodiments, an expression construct of one type comprises polynucleotide sequence elements encoding one or more viral genes, wherein the viral genes are from one or more virus types (e.g., genes or gene variants from AAV and adenovirus). In some embodiments, viral genes from adenovirus are genes and/or gene variants. In some embodiments, viral genes from adenovirus are one or more of E2A (e.g., E2A DNA Binding Protein (DBP), E4 (e.g., E4 Open Reading Frame (ORF) 2, ORF3, ORF4, ORF6/7), VA, and/or variants thereof.

In some embodiments, expression constructs are used for production of viral vectors (e.g. through cell culture). In some embodiments, expression constructs are contacted with cells in combination with one or more transfection reagents (e.g., chemical transfection reagents). In some embodiments, expression constructs are contacted with cells at particular ratios in combination with one or more transfection reagents. In some embodiments, expression constructs of different types are contacted with cells at particular ratios (e.g., weight ratios) in combination with one or more transfection reagents. In some embodiments, expression constructs of different types are contacted with cells at about a 10:1, 9:1, 8:1, 7:1, 6:1, 5:1, 4:1, 3:1, 2:1, 1.5:1, 1:1, 1:1.5, 1:2, 1:3, 1:4, 1:5, 1:6, 1:7, 1:8, 1:9, or 1:10 ratio (e.g., weight ratio). In some embodiments, a first expression construct comprising one or more viral helper genes and a second expression construct comprising one or more payloads are contacted with cells at about a 10:1, 9:1, 8:1, 7:1, 6:1, 5:1, 4:1, 3:1, 2:1, 1.5:1, 1:1, 1:1.5, 1:2, 1:3, 1:4, 1:5, 1:6, 1:7, 1:8, 1:9, or 1:10 ratio (e.g., weight ratio) of the first expression construct to the second expression construct. In some embodiments, a first expression construct comprising one or more payloads and a second expression construct comprising one or more viral helper genes are contacted with cells at about a 10:1, 9:1, 8:1, 7:1, 6:1, 5:1, 4:1, 3:1, 2:1, 1.5:1, 1:1, 1:1.5, 1:2, 1:3, 1:4, 1:5, 1:6, 1:7, 1:8, 1:9, or 1:10 ratio (e.g., weight ratio) of the first expression construct to the second expression construct. In some embodiments, particular ratios of expression constructs improve production of AAV (e.g., increased viral vector yields, increased packaging efficiency, and/or increased transfection efficiency. In some embodiments, cells are contacted with two or more expression constructs(e.g., sequentially or substantially simultaneously). In some embodiments, three or more expression constructs are contacted with cells. In some embodiments, expression constructs comprise one or more promoters (e.g., one or more exogenous promoters). In some embodiments, promoters are or comprise CMV, RSV, CAG, EF1alpha, PGK, A1AT, C5-12, MCK, desmin, p5, p40, or combinations thereof. In some embodiments, expression constructs comprise one or more promoters upstream of a particular polynucleotide sequence element (e.g., a rep or cap gene or gene variant). In some embodiments, expression constructs comprise one or more promoters downstream of a particular polynucleotide sequence element (e.g., a rep or cap gene or gene variant).

In some embodiments, expression constructs comprise one or more polynucleotide sequences encoding elements (e.g., selection markers, origins of replication) necessary for cell culture (e.g., bacterial cell culture, mammalian cell culture). In some embodiments, expression constructs comprise one or more polynucleotide sequences encoding antibiotic resistance genes (e.g., kanamycin resistance genes, ampicillin resistance genes). In some embodiments, expression constructs comprise one or more polynucleotide sequences encoding a bacterial original of replication (e.g., colE1 origin of replication).

In some embodiments, expression constructs comprise one or more transcription termination sequences (e.g., a polyA sequence). In some embodiments, expression constructs comprise one or more of BGH polyA, FIX polyA, SV40 polyA, synthetic polyA, or combinations thereof. In some embodiments, expression constructs comprise one or more transcription termination sequences downstream of a particular sequence element (e.g., a rep or cap gene or gene variant). In some embodiments, expression constructs comprise one or more transcription termination sequences upstream of a particular sequence element (e.g., a rep or cap gene or gene variant).

In some embodiments, expression constructs comprise one or more intron sequences. In some embodiments, expression constructs comprise one or more of introns of different origins (e.g., known genes), including but not limited to FIX intron, Albumin intron, or combinations thereof. In some embodiments, expression constructs comprise one or more introns of different lengths (e.g., 133 bp to 4 kb). In some embodiments, expression constructs comprise one or more intron sequences upstream of a particular sequence element (e.g., a rep or cap gene or gene variant). In some embodiments, expression constructs comprise one or more intron sequences within a particular sequence element (e.g., a rep or cap gene or gene variant). In some embodiments, expression constructs comprise one or more intron sequences downstream of particular sequence element (e.g., a rep or cap gene or gene variant). In some embodiments, expression constructs comprise one or more intron sequences after a promoter (e.g., a p5 promoter). In some embodiments, expression constructs comprise one or more intron sequences before a rep gene or gene variant. In some embodiments, expression constructs comprise one or more intron sequences between a promoter and a rep gene or gene variant.

### Methods of Characterizing AAV Viral Vectors

In accordance with various embodiments, viral vectors may be characterized through assessment of various characteristics and/or features. In some embodiments, assessment of viral vectors can be conducted at various points in a production process. In some embodiments, assessment of viral vectors can be conducted after completion of upstream production steps. In some embodiments, assessment of viral vectors can be conducted after completion of downstream production steps.

### Viral Yields

In some embodiments, characterization of viral vectors comprises assessment of viral yields (e.g., viral titer). In some embodiments, characterization of viral vectors comprises assessment of viral yields prior to purification and/or filtration. In some embodiments, characterization of viral vectors comprises assessment of viral yields after purification and/or filtration. In some embodiments, characterization of viral vectors comprises assessing whether viral yield is greater than or equal to 1e10 vg/mL.

In some embodiments, characterization of viral vectors comprises assessing whether viral yield in crude cell lysates is greater than or equal to 1e11 vg/mL. In some embodiments, characterization of viral vectors comprises assessing whether viral yield in crude cell lysates is greater than or equal to 5e11 vg/mL. In some embodiments, characterization of viral vectors comprises assessing whether viral yield in crude cell lysates is greater than or equal to 1e12 vg/mL. In some embodiments, characterization of viral vectors comprises assessing whether viral yield in crude lysates is between 5e9 vg/mL and 5e11 vg/mL. In some embodiments, characterization of viral vectors comprises assessing whether viral yield in crude lysates is between 5e9 vg/mL and 1e10 vg/mL. In some embodiments, characterization of viral vectors comprises assessing whether viral yield in crude lysates is between 1e10 vg/mL and 1e11 vg/mL. In some embodiments, characterization of viral vectors comprises assessing whether viral yield in crude lysates is between 1e11 vg/mL and 1e12 vg/mL. In some embodiments, characterization of viral vectors comprises assessing whether viral yield in crude lysates is between 1e12 vg/mL and 1e13 vg/mL.

In some embodiments, characterization of viral vectors comprises assessing whether viral yield in purified drug product is greater than or equal to 1e11 vg/mL. In some embodiments, characterization of viral vectors comprises assessing whether viral yield in purified drug product is greater than or equal to 1e12 vg/mL. In some embodiments, characterization of viral vectors comprises assessing whether viral yield in purified drug product is between 1e10 vg/mL and 1e15 vg/mL. In some embodiments, characterization of viral vectors comprises assessing whether viral yield in purified drug product is between Tell vg/mL and 1e15 vg/mL. In some embodiments, characterization of viral vectors comprises assessing whether viral yield in purified drug product is between 1e12 vg/mL and 1e14 vg/mL. In some embodiments, characterization of viral vectors comprises assessing whether viral yield in purified drug product is between 1e13 and 1e14 vg/mL.

In some embodiments, methods and compositions provided herein can provide comparable or increased viral vector yields as compared to previous methods known in the art. For example, in some embodiments, provided methods for producing and/or manufacturing viral vectors comprising use of a two-plasmid transfection system provide comparable or increased viral vector yields as compared to a three-plasmid system. In some embodiments, provided methods for producing and/or manufacturing viral vectors comprising use of a two-plasmid transfection system with particular combinations of sequence elements provide comparable or increased viral vector yields as compared to a two-plasmid system with a different combination of sequence elements. In some embodiments, provided methods for producing and/or manufacturing viral vectors comprising use of a two-plasmid transfection system with particular plasmid ratios provide comparable or increased viral vector yields as compared to a two-plasmid system with different plasmid ratios.

### Viral Packaging

In some embodiments, characterization of viral vectors comprises assessment of viral packaging efficiency (e.g., percent of full versus empty capsids). In some embodiments, characterization of viral vectors comprises assessment of viral packaging efficiency prior to purification and/or full capsid enrichment (e.g., cesium chloride-based density gradient, iodixanol-based density gradient or ion exchange chromatography). In some embodiments, characterization of viral vectors comprises assessing whether viral packaging efficiency is greater than or equal to 20% prior to purification and/or filtration (e.g., 20%, 25%, 30%, 35%, 40%, 45%, 50%, 55%, 60%, 65%, 70%, 75%, 80%, 85%, 90%, 95%, 99%, 100%). In some embodiments, characterization of viral vectors comprises assessment of viral packaging efficiency after purification and/or full capsid enrichment. In some embodiments, characterization of viral vectors comprises assessing whether viral packaging efficiency is greater than or equal to 50% after purification and/or filtration (e.g., 50%, 55%, 60%, 65%, 70%, 75%, 80%, 85%, 90%, 95%, 99%, 100%).

In some embodiments, methods and compositions provided herein can provide comparable or increased packaging efficiency as compared to previous methods known in the art. For example, in some embodiments, provided methods for producing and/or manufacturing viral vectors comprising use of a two-plasmid transfection system provide comparable or increased packaging efficiency as compared to a three-plasmid system. In some embodiments, provided methods for producing and/or manufacturing viral vectors comprising use of a two-plasmid transfection system with particular combinations of sequence elements provide comparable or increased packaging efficiency as compared to a two-plasmid system with a different combination of sequence elements. In some embodiments, provided methods for producing and/or manufacturing viral vectors comprising use of a two-plasmid transfection system with particular plasmid ratios provide comparable or increased packaging efficiency as compared to a two-plasmid system with different plasmid ratios.

### Replication Competent Vector Levels

In some embodiments, characterization of viral vectors comprises assessment of levels of replication competent vectors. In some embodiments, characterization of viral vectors comprises assessment of levels of replication competent vectors prior to purification and/or filtration. In some embodiments, characterization of viral vectors comprises assessment of levels of replication competent vectors after purification and/or filtration. In some embodiments, characterization of viral vectors comprises assessing whether replication competent vector levels are less than or equal to 1 rcAAV in 1E10 vg.

In some embodiments, methods and compositions provided herein can provide comparable or reduced replication competent vector levels as compared to previous methods known in the art. For example, in some embodiments, provided methods for producing viral vectors comprising use of a two-plasmid transfection system provide comparable or reduced replication competent vector levels as compared to a three-plasmid system. In some embodiments, provided methods for producing viral vectors comprising use of a two-plasmid transfection system with particular combinations of sequence elements provide comparable or reduced replication competent vector levels as compared to a two-plasmid system with a different combination of sequence elements. In some embodiments, provided methods for producing viral vectors comprise use of a two-plasmid transfection system with one or more intronic sequences inserted in the rep gene provide comparable or reduced replication competent vector levels as compared to a two-plasmid system without said intronic sequence(s).

### Exemplification

### Example 1: Materials and Methods

### Animals

Animal handling and all experimental procedures were conducted in accordance with the guidelines of the Institutional Animal Care and Use Committee of LogicBio Therapeutics. FvB or C57BL/6 animals (referred as wild-type mice) were purchased from Jackson lab (Bar Harbor, ME) and PXB animals were purchased from Phoenix Bio (Hiroshima, Japan). All animals were acclimatized for a minimum of 5 days before the study. The B6.129-Ugt1tm1Rhtu/J mouse model (also denoted as Ugt1^{-/-}) mice used in the study were described previously. Breeders of Ugt1^{-/-} mice were obtained from Jackson lab (Bar Harbor, ME) after 9 backcrosses with C57Bl/6 WT mice. All animals were housed in a facility with controlled temperature (23±3° C.) and humility (50%±20%) with a 12-h light/dark cycle, and received standard diet and water ad libitum. For vector administration, animals were given rAAV vector compositions with balanced (same length) or unbalanced (different length) homology arm designs at dosages of 3e13, 1e14, 2e14, or 3E14 vg/kg. Neonatal animals were given the vector via temporal vein; pediatric animals (14-days postnatal, PND14) were given by retro-orbital injection; pediatric animals of PND28 (28-days postnatal) to adult age were introduced via lateral tail vein. Blood samples were collected via facial vein during the study period at several time points. At the conclusion of study, animals were euthanized and blood samples were collected via cardiac puncture. Liver was harvested and flash frozen.

### Phototherapy

Newborns of Ugt1^{-/-} mice were exposed to blue fluorescent light (λ=450 nm; averagely 25 μW/cm2/nm; Philips TL 20 W/52 lamps) for 12 h/days (synchronized with the light period of the light-dark cycle) up to 21 days after birth, and then maintained under normal light conditions. Intensity of the lamps will be monitored monthly with an ILT74 Hyperbilirubinemia Light Meter (International Light Technologies).

### Example 2: Uneven Homology Arms May Improve Editing Activity in Mice

The present example demonstrates that, among other things, viral vectors comprising unbalanced homology arms may improve editing activity in a mouse model system.

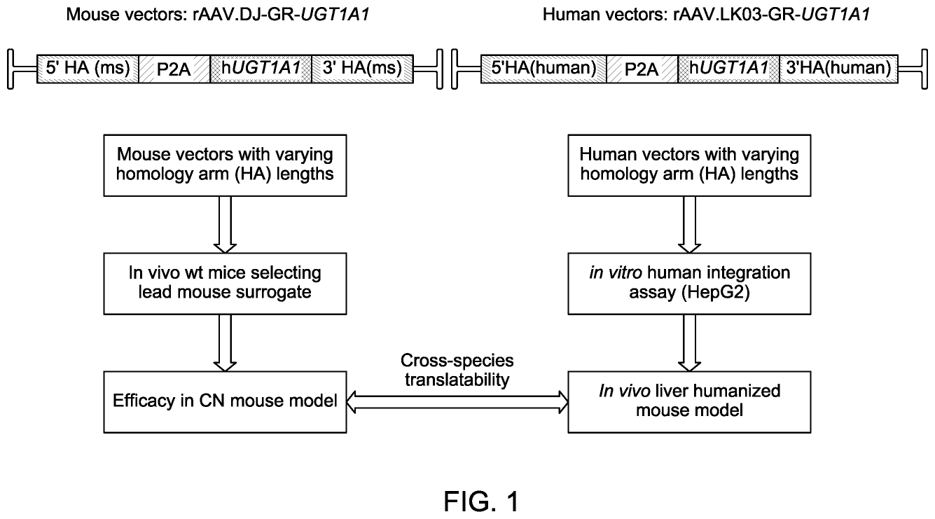

Viral vectors comprising an AAV-DJ viral capsid, human UGT1A1 (hUGT1A1) transgene, P2A sequence, and flanking homology arms were constructed (FIG. 1). Viral vector compositions were administered to C57Bl/6 wild-type mice at 3E13 vg/kg dosage at 28 days postnatal (PND28). Levels of fused mRNA and ALB-2A biomarker were measured to determine GeneRide activity. Fixed liver samples were sectioned and stained against hUGT1A1 transgene (the antibody is specific to human UGT1A1 protein and does not cross-react to endogenous mouse UGT1A1) to identify the presence of GeneRide-edited hepatocytes (FIG. 2). Shown in FIG. 2 panels A and B are representative IHC images from animals treated with 1.0/1.0 vector and 1.0/1.6 vector, respectively. The brown spots represent GeneRide-edited hepatocytes that are expressing hUGT1A1 transgene. The images were analyzed in a blind fashion using a semi-quantitative algorithm (ImageJ, NIH) to calculate the frequency of hepatocyte editing.

Tested vectors comprised homology arms of various lengths. As shown in FIG. 2, a construct denoted as 1.0/1.6 comprises a 5′ homology arm 1000 nt in length and a 3′ homology arm 1600 nt in length.

Among other things, the present disclosure demonstrates that certain configurations of homology arms of differing lengths may improve editing activity in animals (e.g., mice). In some embodiments, as demonstrated in FIG. 2, viral vectors comprising unbalanced homology arms may provide improved editing activity relative to viral vectors comprising balanced homology arms. In some embodiments, viral vectors may comprise one or more transgenes (e.g., UGT1A1).

### Example 3: Viral Vector Compositions May Offer Increased Editing Activity at Particular Timepoints

The present example demonstrates that, among other things, a viral vector composition may improve editing activity in a mouse model system when administered at specific timepoints.

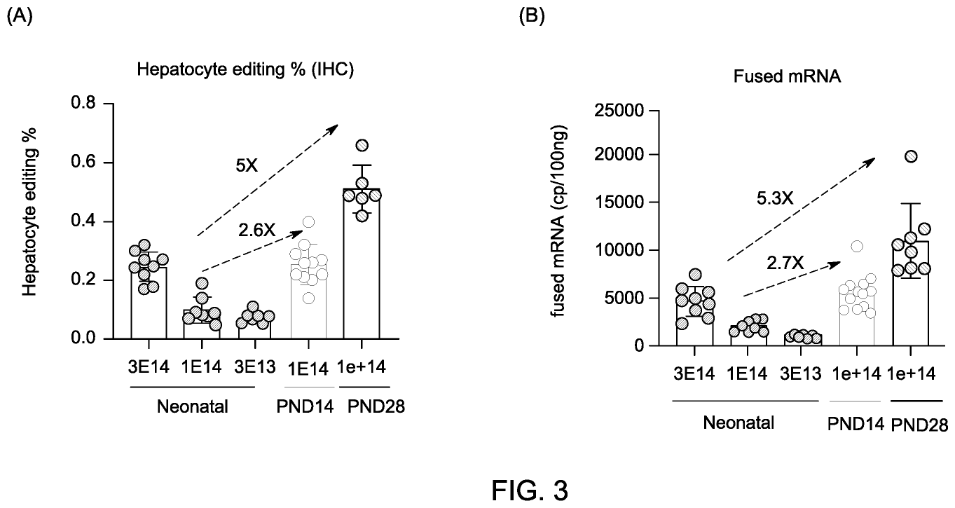

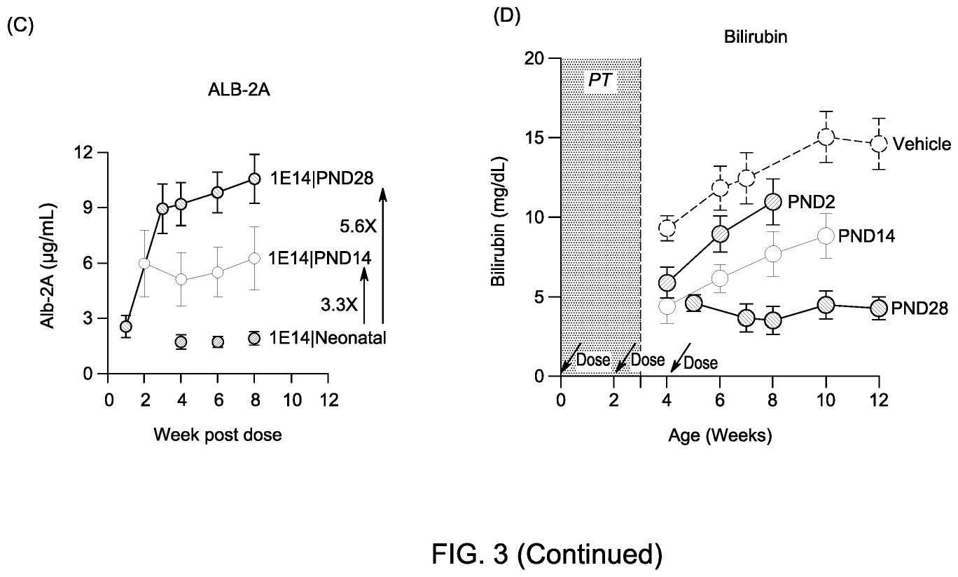

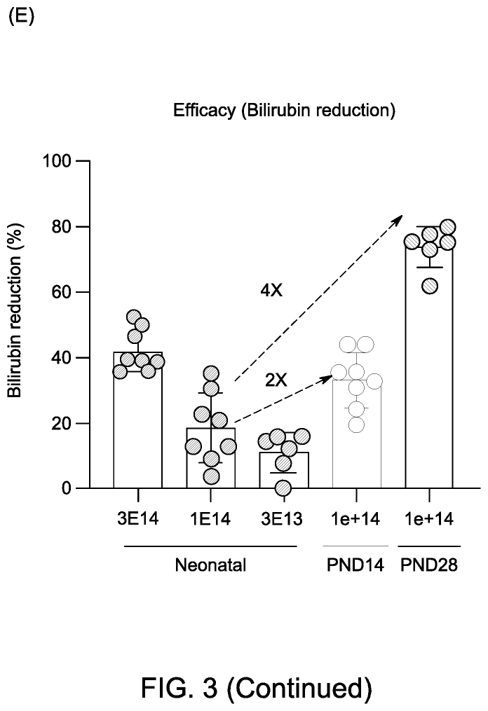

A viral vector comprising an AAV-DJ viral capsid, mouse UGT1A1 (mUGT1A1) transgene, P2A sequence, a 5′ homology arm 1000 nt in length and a 3′ homology arm 1600 nt in length was constructed (FIG. 3). Viral vector compositions were administered at various dosages (vg/kg) to neonatal, 14-days postnatal (PND14), and 28-days postnatal (PND28) Ugt1^{-/-} mice. Levels of fused mRNA and ALB-2A biomarker were measured to determine editing activity. Mouse hepatocytes were also stained with mUGT1A1 (un-edited hepatocytes are negative in mUGT1A1 staining due to Ugt1 KO) to identify presence of GeneRide-edited hepatocytes. The images were analyzed in a blind fashion using a semi-quantitative algorithm (ImageJ, NIH) to calculate the frequency of hepatocyte editing. Bilirubin levels (efficacy biomarker) were measured throughout the course of the experiment.

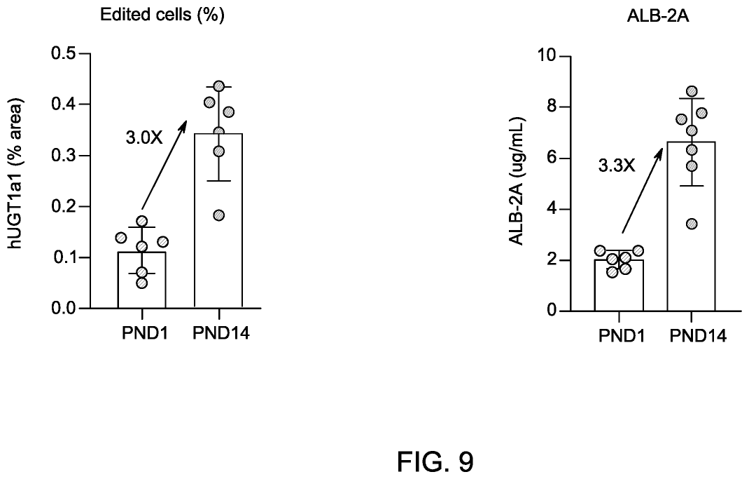

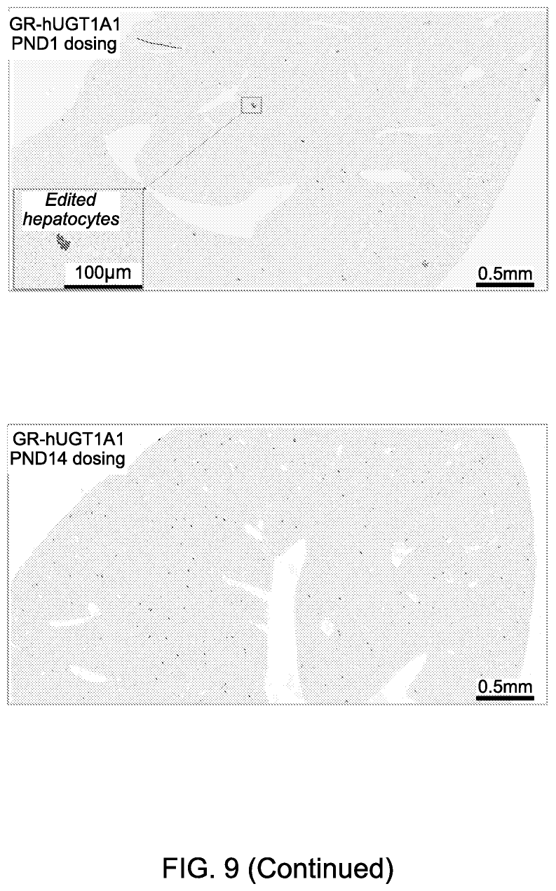

A viral vector comprising an AAV-DJ viral capsid, human UGT1A1 (hUGT1A1) transgene, P2A sequence, a 5′ homology arm 1300 nt in length and a 3′ homology arm 1400 nt in length was constructed (FIG. 9). Viral vector compositions were administered at a dosage of 3E13 vg/kg to neonatal (PND1) and 14-days postnatal (PND14), C57B6 mice. Mouse hepatocytes were stained with hUGT1A1 to identify presence of GeneRide-edited hepatocytes and calculate percentage of edited cells.

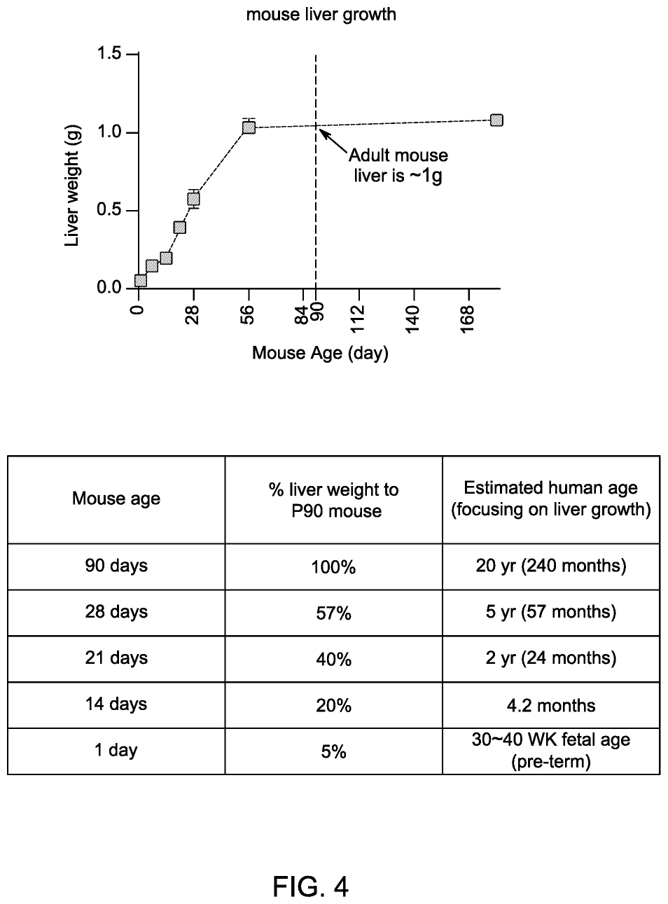

Among other things, the present disclosure demonstrates that viral vectors may provide improved editing activity when administered at a particular postnatal timepoint (e.g., PND14 or PND28) as compared to neonatal administration. Furthermore, administration of viral vectors comprising uneven homology arms at a particular timepoint may provide synergistic improvements in gene editing efficiency. As demonstrated in FIG. 3, administration of viral vectors with a 5′ homology arm 1000 nt in length and a 3′ homology arm 1600 nt in length at PND14 and PND28 displayed increased editing activity and reduced levels of bilirubin over the course of 12 weeks. As shown in FIG. 9, administration of viral vectors with a 5′ homology arm 1300 nt in length and a 3′ homology arm 1400 nt in length displayed increased editing activity when administered at PND14 as compared to a neonatal (PND1) timepoint. These dosing timepoints may inform timing of dosing in future human experiments, as mouse age may be correlated with human age based upon percentage of liver weight at each timepoint (FIG. 4).

### Example 4: Uneven Homology Arms May Improve Editing Activity in Humans

The present example demonstrates that, among other things, viral vectors comprising unbalanced homology arms may improve editing activity in human cells.

Viral vectors comprising an AAV-LK03 viral capsid, human UGT1A1 (hUGT1A1) transgene, P2A sequence, and flanking homology arms were constructed (FIG. 1). Viral vector compositions were administered to a human cell line (HepG2) in vitro (FIG. 5, panel A) or a liver humanized PXB mouse model (FIG. 5, panel B). Levels of fused mRNA were measured for in vitro experiments and genomic DNA (gDNA) integration rates were measured for PXB mouse experiments.

Tested vectors comprised homology arms of various lengths. As shown in FIG. 5, a construct denoted as 1.0/1.6 comprises a 5′ homology arm 1000 nt in length and a 3′ homology arm 1600 nt in length.

Among other things, the present disclosure demonstrates that certain configurations of homology arms of differing lengths may improve editing activity in humans. In some embodiments, as demonstrated in FIG. 5, viral vectors comprising unbalanced homology arms may provide improved editing activity relative to viral vectors comprising balanced homology arms.

### Example 5: Uneven Homology Arms May Improve Editing Efficiency Across Different Species

The present example demonstrates that, among other things, viral vectors of the present disclosure comprising unbalanced homology arms may provide editing activity in different species or model systems for different species (e.g., mice and humans).

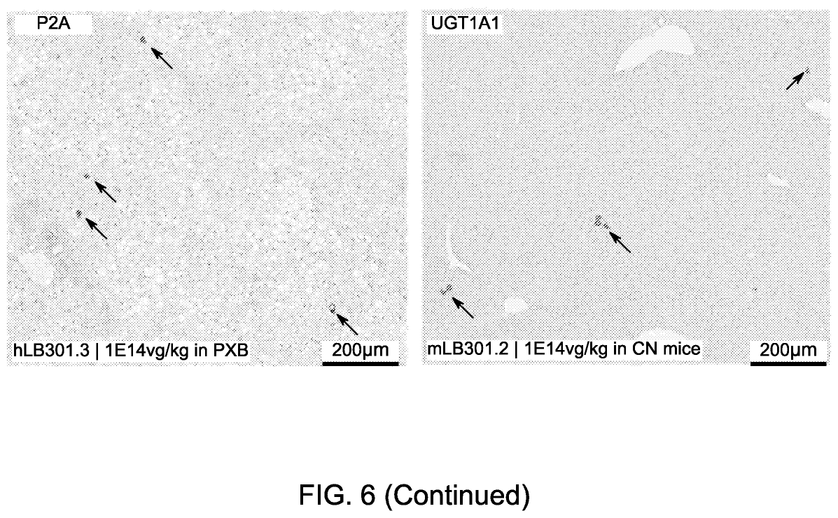

Viral vectors comprising a human UGT1A1 (hUGT1A1) transgene, P2A sequence, and flanking homology arms were constructed (FIG. 1). Vectors for administration in Ugt1^{-/-} mice comprised an AAV-DJ viral capsid, a 5′ homology arm 1000 nt in length, and a 3′ homology arm 1600 nt in length. Vectors for administration in humanized PXB mice comprised an AAV-LK03 capsid, a 5′ homology arm 1600 nt in length, and a 3′ homology arm 1000 nt in length. Viral vector compositions were administered to Ugt1^{-/-} or PXB mice at indicated concentrations (vg/kg). Levels of gDNA integration and UGT1A1 staining were measured (FIG. 6).

Among other things, the present disclosure demonstrates that viral vector compositions as disclosed herein may provide comparable editing activity in different species or model systems for different species (e.g., mice and humans). In some embodiments, as demonstrated in FIG. 6, viral vectors comprising specific configurations of unbalanced homology arms may provide improved editing activity in different species or model systems for different species (e.g., mice and humans). In some embodiments, viral vector compositions optimized for expression in one species or a model system for one species may differ from viral vector compositions optimized for expression in a different species or a model system for a different species (e.g., vectors may comprise different combination of homology arm lengths).

### Example 6: Homology Arm Length May Affect Editing Activity

The present example demonstrates that, among other things, viral vectors of the present disclosure comprising homology arms of certain lengths may provide editing activity.

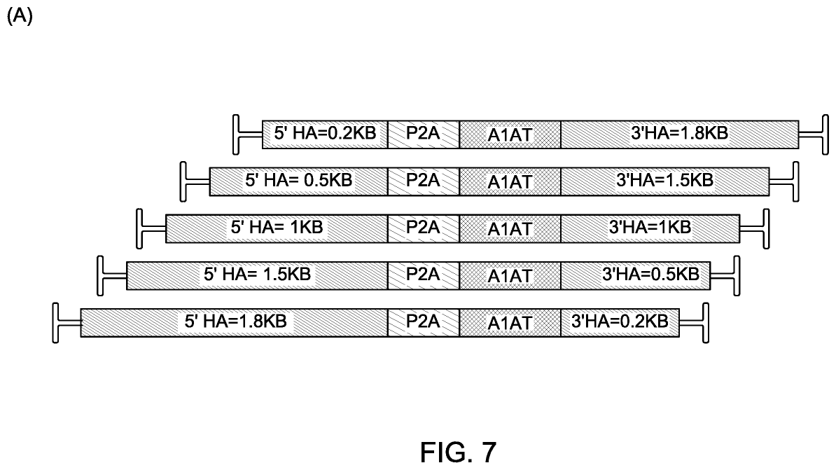

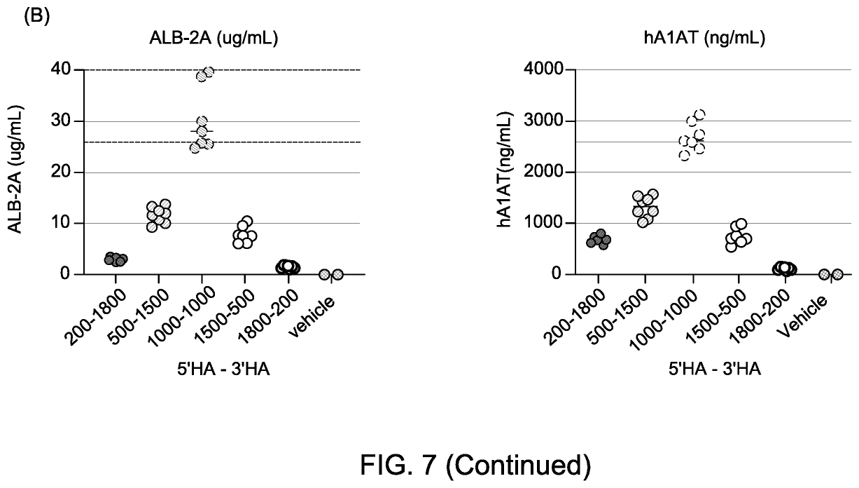

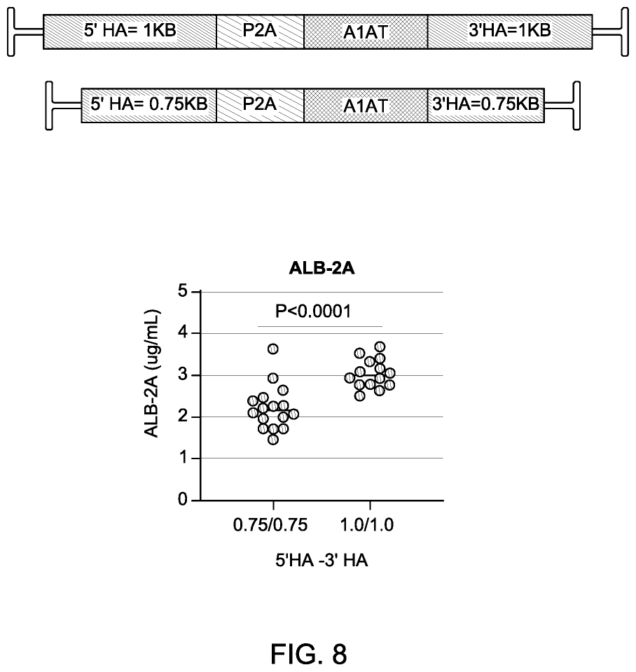

Viral vectors comprising a human A1AT (hA1AT) transgene, P2A sequence, and flanking homology arms were constructed (FIG. 7, FIG. 8). Vectors for administration in FvB wild-type mice comprised an AAV-DJ viral capsid, and homology arms of various lengths, as indicated in FIGS. 7 and 8. Viral vector compositions were administered 1E14 vg/kg dosage. Levels of ALB-2A biomarker (FIGS. 7 and 8) and hA1AT were measured (FIG. 7).

Among other things, the present disclosure demonstrates that viral vectors comprising homology arms may demonstrate reduced editing activity when at least one homology arm is below a certain length (e.g., less than or equal to 750 nt). In some embodiments, as demonstrated in FIG. 7, unbalanced homology arm designs with at least one arm of length 500 nt or less displayed reduced editing efficiency compared to a balanced homology arm design with 1000 nt lengths. Additionally, as demonstrated in FIG. 8, in some embodiments a balanced homology arm design with 1000 nt lengths displayed improved editing efficiency compared to a balanced homology arm design with 750 nt lengths.

### Example 7: Comparison of the Impact of Homology Arm on GENERIDE Activity

The present example confirms that, among other things, viral vectors of the present disclosure comprising homology arms of certain lengths (or ratios of certain lengths) may provide enhanced editing activity as compared to viral vectors that do not comprise such homology arms.

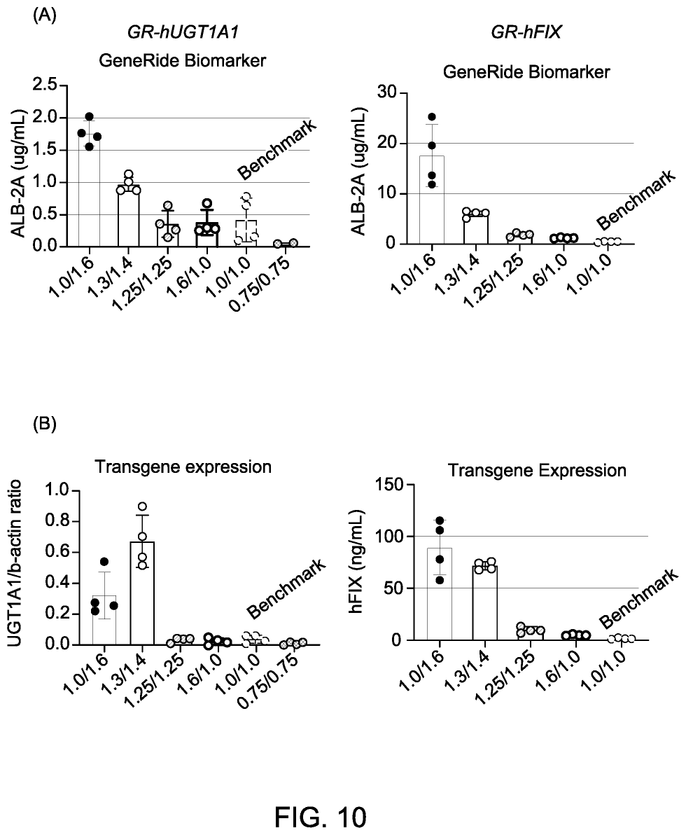

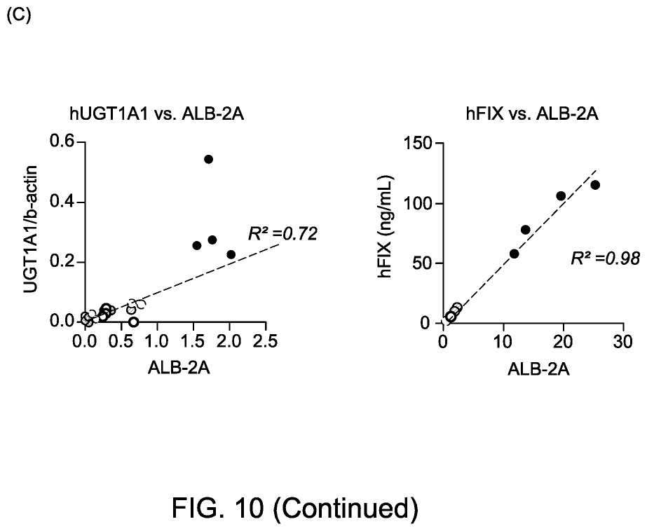

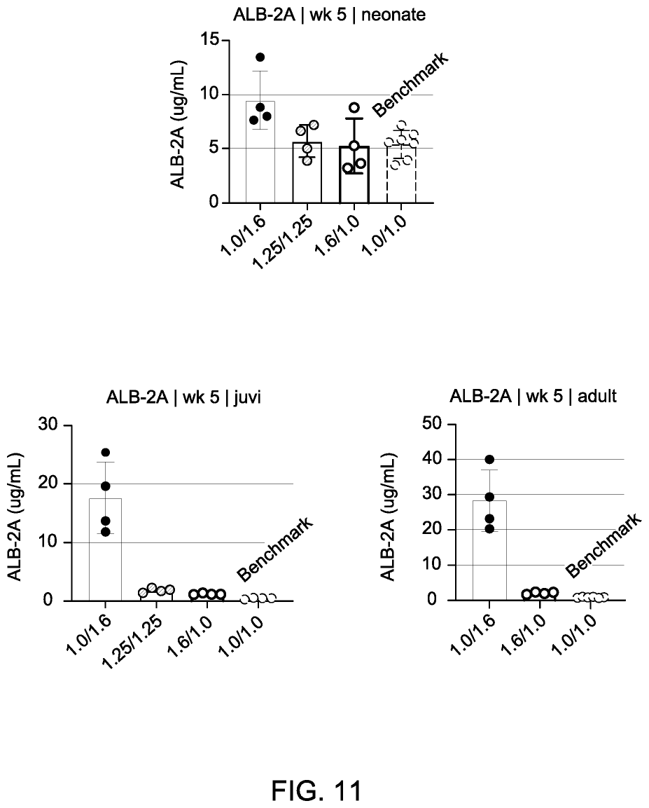

Viral vectors comprising a human UGT1A1 (hUGT1A1) or FIX (hFIX) transgene, P2A sequence, and flanking homology arms were constructed (see FIG. 10). Vectors for administration in mice comprised an AAV-DJ viral capsid, and homology arms of various lengths, as indicated in FIG. 10. Viral vector compositions were administered at an appropriate dosage. Levels of ALB-2A biomarker and transgene (e.g., hUGT1A1 and/or hFIX) to actin ratios were measured (FIG. 10, panels A-B). Transgene expression levels were compared to ALB-2A levels (FIG. 10, panel C).

Among other things, this example demonstrates that certain configurations of homology arms of differing lengths may improve editing activity in animals (e.g., mice). In some embodiments, as demonstrated in FIG. 10, viral vectors comprising unbalanced homology arms may provide improved editing activity relative to viral vectors comprising balanced homology arms. In some embodiments, as demonstrated in FIG. 10, a GeneRide vector comprising a 5′ 1000 nt and a 3′ 1600 nt homology arm flanking a human UGT1A1 and/or FIX transgene may provide improved editing activity (as measured by ALB-2A levels and/or transgene (UGT1A1 and/or FIX) expression) to actin ratios in animals (e.g., mice), relative to viral vectors comprising balanced homology arms.

### Example 8: Uneven Homology Arms May Improve Editing Activity in Older Mice

The present example confirms, among other things, viral vectors comprising unbalanced homology arms may improve editing activity in a mouse model system when administered at specific timepoints (e.g., older dosing age) as compared to viral vectors that do not comprise such homology arms.

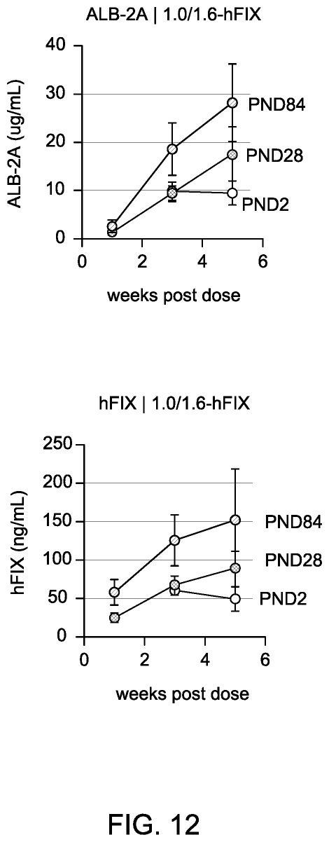

Viral vectors comprising an AAV-DJ viral capsid, human FIX (hFIX) transgene, P2A sequence, and flanking homology arms were constructed. Vectors for administration in mice comprised an AAV-DJ viral capsid, and homology arms of various lengths, as indicated in FIG. 11. Viral vector compositions were administered at 3e13 vg/kg to mice of varying ages. Editing activity was determined by measuring levels of ALB-2A biomarker (e.g., GENERIDE biomarker) and/or transgene expression (FIG. 12).

Among other things, this example demonstrates that viral vectors comprising unbalanced homology arms may provide improved editing activity when administered at a particular postnatal time point (e.g., juvenile and/or adult) as compared to neonatal administration. Furthermore, as demonstrated in FIG. 11 and FIG. 12, administration of viral vectors comprising a 5′ 1000 nt and a 3′ 1600 nt homology arm flanking a human (e.g. FIX) transgene at a particular postnatal time point (e.g., juvenile and/or adult) may provide synergistic improvements in gene editing efficiency in animals (e.g., mice), as compared to neonatal administration. In some embodiments, as demonstrated in FIG. 12, administration of viral vectors comprising a 5′ 1000 nt and a 3′ 1600 nt homology arm flanking a human (e.g. FIX) transgene at a particular postnatal time point (e.g., PND84) may provide improvements in gene editing efficiency at least 3 weeks post dose in animals (e.g., mice), as compared to neonatal administration. In some embodiments, as demonstrated in FIG. 12, administration of viral vectors comprising a 5′ 1000 nt and a 3′ 1600 nt homology arm flanking a human (e.g. FIX) transgene at a particular postnatal time point (e.g., PND84) may provide sustained improvements in gene editing efficiency for at least 6 weeks post dose in animals (e.g., mice), as compared to neonatal administration.

In some embodiments, as shown in FIG. 4, these dosing time points may inform timing of dosing in future human experiments, as mouse age may be correlated with human age based upon percentage of liver weight at each time point.

### Example 9: Uneven Homology Arms May Improve Liver Function In Vivo

This example demonstrates that, among other things, viral vectors comprising unbalanced homology arms flanking a sequence encoding fumarylacetoacetate hydrolase (FAH) may be used to treat or prevent tyrosinemia in vivo (e.g., in one or more mouse models).

### Materials and Methods

### Animal Study

FAH knock out (Fah−/−, KO) and heterozygous Fah+/− littermates (HET) animals are purchased from Jackson Laboratories. FRG mice were purchased from Yecuris corporation.

### GeneRide Proof of Concept (PoC) in Tyrosinemia FRG Mice

Four-week-old FRG male animals were treated with either vehicle or rAAV.DJ-GR-hFAH at 1e14 vg/kg via retro-orbital sinus under anesthesia. All mice were kept on 8 mg/L of Nitisinone (NTBC) prior to the initiation of the study and one week post dosing, and then NTBC were cycled up to 5 weeks post dosing based on body weight loss and then NTBC was discontinued. During the study, animals were sampled periodically by submandibular bleed and plasma was collected and stored at −80° C. until further analysis. Terminal harvest was conducted at week 9 and 16 post dosing. At sacrifices, blood was collected for plasma via cardiac puncture. Animal were either dissected the whole liver or underwent liver perfusion to collect hepatocyte. For animals which the whole liver was dissected, one lobe of liver was fixed 10% formalin, and the remaining was snap-frozen and stored at −80° C. Next day, formalin-fixed liver was transferred to 70% ethanol for paraffin embedding. Following the liver perfusion, isolated hepatocytes were centrifuged at 300×g for 5 min at 4° C. and stored at −80° C.

### Dose-Response PoC of GeneRide in FAH Mice

Pediatric Fah−/− (KO) and Fah+/−(HET) animals at 14 days old were treated with rAAV.DJ-GR-mFAH at dosages of 1e13, 3e13, or 1e14 vg/kg via retro-orbital sinus under anesthesia. All mice were kept on 8 mg/L of NTBC prior to the initiation of the study and one week post dosing, and then NTBC were cycled up to 2 weeks post dosing based on body weight loss. During the study, animals were sampled periodically by submandibular bleed and plasma was collected and stored at −80° C. until further analysis. Terminal harvest was conducted at 16 weeks post dosing. At sacrifices, blood was collected for plasma via cardiac puncture. Animal underwent liver perfusion to collect hepatocyte. One lobe of liver was sutured and dissected for formalin fixation prior perfusion began. Following the liver perfusion, isolated hepatocytes were centrifuged at 300×g for 5 min at 4° C. and stored at −80° C. Next day, formalin-fixed liver was transferred to 70% ethanol for paraffin embedding.

### Assessment of Hepatocellular Carcinoma (HCC) Risk Between NTBC Vs GeneRide

Fah−/− animals were maintained on 8 mg/L NTBC since birth. At Four-week-old age, a group of Fah−/− animals were randomly selected and treated with rAAV.DJ-GR-mFAH at dosages of 1e14 vg/kg via retro-orbital sinus under anesthesia and then withdrawn from NTBC (GeneRide treatment group). Another group of Fah−/− animals were maintained on 8 mg/L NTBC (Standard of Care group). A third group of Fah+/− littermates were enrolled in the study but not receiving any treatment (no NTBC or GeneRide). All animals were followed up till one year of age and HCC biomarker (AFP level) were assessed periodically.

### Assessment of Compatibility Between NTBC (Standard of Care) and GeneRide

Four-week-old Fah−/− animals were treated with rAAV.DJ-GR-mFAH at dosages of 1e14 vg/kg via retro-orbital sinus under anesthesia. All mice were kept on 8 mg/L of NTBC prior to the initiation of the study until 4 weeks post dosing, and then NTBC were either maintained at 8 mg/L (control) or titrated down to 3 mg/L, 0.8 mg/L, or 0.3 mg/L for 8 weeks. During the study, animals were sampled periodically by submandibular bleed and plasma was collected and stored at −80° C. until further analysis. At sacrifices, blood was collected for plasma via cardiac puncture. For liver dissection, one lobe of liver was fixed by 10% formalin, and the remaining was snap-frozen and stored at −80° C. Next day, formalin-fixed liver was transferred to 70% ethanol for paraffin embedding.

### Targeted Genomic DNA Integration in Liver

Genomic DNA was extracted from frozen liver tissues and targeted genomic DNA integration was analyzed by long-range polymerase chain reaction (PCR) amplification, followed by quantitative polymerase chain reaction (qPCR) quantification using a qualified method (see below figure). Long Range PCR was performed using a forward primer (F1) and a reverse primer (R1). The PCR product was cleaned by solid phase reversible immobilization beads (ABM, G950) and used as template for qPCR using the forward primer (F1), a reverse primer (R2) and a probe (P1). The primers and probes are (F1) 5′-ATGTTCCACGAAGAAGCCA-3′, (R1) 5′-TCAGCAGGCTGAAATTGGT-3, (R2) 5′-AGCTGTTTCTTACTCCATTCTCA-3′, (P1) 5′-AGGCAACGTCATGGGTGTGACTTT-3′. The mouse transferrin receptor (Tfrc) was used as an internal control in qPCR.

### Plasma Albumin-2A Fusion Protein Quantification

Mouse Albumin-2A in plasma was measured by chemoluminescence ELISA, using a proprietary rabbit polyclonal anti-2A antibody for capture and an HRP-labeled polyclonal goat anti-mouse Albumin antibody (abeam ab19195) for detection. Recombinant mouse Albumin-2A expressed in mammalian cells and affinity-purified was used to build the standard curve in 1% control mouse plasma to account for matrix effects. Milk at 1% (Cell Signaling 9999S) in PBS was used for blocking and BSA at 1% for sample dilution in PBST.

### Plasma Liver Injury Biomarkers Quantification

Alanine aminotransferase (ALT) activity and total bilirubin level in mouse plasma were quantified as biomarkers for liver injury. Plasma ALT activity was quantified using an alanine aminotransferase activity colorimetric assay kit (BioVision) following the vendor's instructions. Total bilirubin in plasma was measured using a certified clinical analyzer, Advanced® BR2 Bilirubin Stat-Analyzer™ (Advanced Instruments, LLC) according to the manufacturer's protocol.

### Plasma Alpha-Fetoprotein Quantification

Plasma alpha-fetoprotein was quantified using chemoluminescence ELISA kit (R&D Systems) according to the manufacturer's protocol.

### Immunohistochemistry

Immunohistochemistry was performed on a robotic platform (Ventana discover Ultra Staining Module, Ventana Co., Tucson, AZ). Tissue sections (4 μm) were deparaffinized and underwent heat-induced antigen retrieval for 64 min. Endogenous peroxidases were blocked with peroxidase inhibitor (CM1) for 8 min before incubating the section with anti-FAH antibody (Yecuris, Portland, OR) at 1:400 dilution for 60 min at room temperature. Antigen-antibody complex was then detected using DISC. OmniMap anti-rabbit multimer RUO detection system and DISCOVERY ChromoMap DAB Kit Ventana Co., Tucson, AZ). All the slides were counterstained with hematoxylin (Fisher Sci, Waltham, MA) subsequently, dehydrated, cleared and mounted for image scanning using digital slide scanner (Hamamatsu, Bridgewater, NJ). Scanned images were evaluated in a blinded fashion using ImageJ software to quantify the area of positive staining.

### Exemplary Sequences Used in the Present Examples and in Example 10 are Provided Below:

SEQ ID NO. 1: AAV-DJ-mha-mFAH (PM-0550)
cagatcctctacgccggacgcatcgtggccggcatcaccggcgccacaggtgcggttgct
ggcgcctatatcgccgacatcaccgatggggaagatcgggctcgccacttcgggctcatg
agcgcttgtttcggcgtgggtatggtggcaggccccgtggccgggggactgttgggcgcc
atctccttgcatgcaccattccttgcggcggcggtgctcaacggcctcaacctactactg
ggctgcttcctaatgcaggagtcgcataagggagagcgtcgaatggtgcactctcagtac
aatctgctctgatgccgcatagttaagccagccccgacacccgccaacacccgctgacgc
gccctgacgggcttgtctgctcccggcatccgcttacagacaagctgtgaccgtctccgg
gagctgcatgtgtcagaggttttcaccgtcatcaccgaaacgcgcgagacgaaagggcct
cgtgatacgcctatttttataggttaatgtcatgataataatggtttcttagacgtcagg
tggcacttttcggggaaatgtgcgcggaacccctatttgtttatttttctaaatacattc
aaatatgtatccgctcatgagacaataaccctgataaatgcttcaataatattgaaaaag
gaagagtatgagccatattcaacgggaaacgtcgaggccgcgattaaattccaacatgga
tgctgatttatatgggtataaatgggctcgcgataatgtcgggcaatcaggtgcgacaat
ctatcgcttgtatgggaagcccgatgcgccagagttgtttctgaaacatggcaaaggtag
cgttgccaatgatgttacagatgagatggtcagactaaactggctgacggaatttatgcc
tcttccgaccatcaagcattttatccgtactcctgatgatgcatggttactcaccactgc
gatccccggaaaaacagcattccaggtattagaagaatatcctgattcaggtgaaaatat
tgttgatgcgctggcagtgttcctgcgccggttgcattcgattcctgtttgtaattgtcc
ttttaacagcgatcgcgtatttcgtctcgctcaggcgcaatcacgaatgaataacggttt
ggttgatgcgagtgattttgatgacgagcgtaatggctggcctgttgaacaagtctggaa
agaaatgcataaacttttgccattctcaccggattcagtcgtcactcatggtgatttctc
acttgataaccttatttttgacgaggggaaattaataggttgtattgatgttggacgagt
cggaatcgcagaccgataccaggatcttgccatcctatggaactgcctcggtgagttttc
tccttcattacagaaacggctttttcaaaaatatggtattgataatcctgatatgaataa
attgcagtttcatttgatgctcgatgagtttttctaactgtcagaccaagtttactcata
tatactttagattgatttaaaacttcatttttaatttaaaaggatctaggtgaagatcct
ttttgataatctcatgaccaaaatcccttaacgtgagttttcgttccactgagcgtcaga
ccccgtagaaaagatcaaaggatcttcttgagatcctttttttctgcgcgtaatctgctg
cttgcaaacaaaaaaaccaccgctaccagcggtggtttgtttgccggatcaagagctacc
aactctttttccgaaggtaactggcttcagcagagcgcagataccaaatactgttcttct
agtgtagccgtagttaggccaccacttcaagaactctgtagcaccgcctacatacctcgc
tctgctaatcctgttaccagtggctgctgccagtggcgataagtcgtgtcttaccgggtt
ggactcaagacgatagttaccggataaggcgcagcggtcgggctgaacggggggttcgtg
cacacagcccagcttggagcgaacgacctacaccgaactgagatacctacagcgtgagct
atgagaaagcgccacgcttcccgaagggagaaaggcggacaggtatccggtaagcggcag
ggtcggaacaggagagcgcacgagggagcttccagggggaaacgcctggtatctttatag
tcctgtcgggtttcgccacctctgacttgagcgtcgatttttgtgatgctcgtcaggggg
gcggagcctatggaaaaacgccagcaacgcggcctttttacggttcctggccttttgctg
gccttttgctcacatgttctttcctgcgttatcccctgattctgtggataaccgtattac
cgcctttgagtgagctgataccgctcgccgcagccgaacgaccgagcgcagcgagtcagt
gagcgaggaagcggaagagcgcccaatacgcaaaccgcctctccccgcgcgttggccgat
tcattaatgcagctgtggaatgtgtgtcagttagggtgtggaaagtccccaggctcccca
gcaggcagaagtatgcaaagcatgcatctcaattagtcagcaaccaggtgtggaaagtcc
ccaggctccccagcaggcagaagtatgcaaagcatgcatctcaattagtcagcaaccata
gtcccgcccctaactccgcccatcccgcccctaactccgcccagttccgcccattctccg
ccccatggctgactaattttttttatttatgcagaggccgaggccgcctcggcctctgag
ctattccagaagtagtgaggaggcttttttggaggcctaggcttttgcaaaaagttggcc
actccctctctgcgcgctcgctcgctcactgaggccgcccgggcaaagcccgggcgtcgg
gcgacctttggtcgcccggcctcagtgagcgagcgagcgcgcagagagggagtggccaac
tccatcactaggggttcctTACGTAATGCATGGATCCCCTAGGgcggccgcCTGAAACTA
GACAAAACCCGTGTGACTGGCATCGATTATTCTATTTGATCTAGCTAGTCCTAGCAAAGT
GACAACTGCTACTCCCCTCCTACACAGCCAAGATTCCTAAGTTGGCAGTGGCATGCTTAA
TCCTCAAAGCCAAAGTTACTTGGCTCCAAGATTTATAGCCTTAAACTGTGGCCTCACATT
CCTTCCTATCTTACTTTCCTGCACTGGGGTAAATGTCTCCTTGCTCTTCTTGCTTTCTGT
CCTACTGCAGGGCTCTTGCTGAGCTGGTGAAGCACAAGCCCAAGGCTACAGCGGAGCAAC
TGAAGACTGTCATGGATGACTTTGCACAGTTCCTGGATACATGTTGCAAGGCTGCTGACA
AGGACACCTGCTTCTCGACTGAGGTCAGAAACGTTTTTGCATTTTGACGATGTTCAGTTT
CCATTTTCTGTGCACGTGGTCAGGTGTAGCTCTCTGGAACTCACACACTGAATAACTCCA
CCAATCTAGATGTTGTTCTCTACGTAACTGTAATAGAAACTGACTTACGTAGCTTTTAAT
TTTTATTTTCTGCCACACTGCTGCCTATTAAATACCTATTATCACTATTTGGTTTCAAAT
TTGTGACACAGAAGAGCATAGTTAGAAATACTTGCAAAGCCTAGAATCATGAACTCATTT
AAACCTTGCCCTGAAATGTTTCTTTTTGAATTGAGTTATTTTACACATGAATGGACAGTT
ACCATTATATATCTGAATCATTTCACATTCCCTCCCATGGCCTAACAACAGTTTATCTTC
TTATTTTGGGCACAACAGATGTCAGAGAGCCTGCTTTAGGAATTCTAAGTAGAACTGTAA
TTAAGCAATGCAAGGCACGTACGTTTACTATGTCATTGCCTATGGCTATGAAGTGCAAAT
CCTAACAGTCCTGCTAATACTTTTCTAACATCCATCATTTCTTTGTTTTCAGGGTCCAAA
CCTTGTCACTAGATGCAAAGACGCCTTAGCCggaagcggcgccaccaatttcagcctgct
gaaacaggccggcgacgtggaagagaaccctggccctTCCTTTATTCCAGTGGCCGAGGA
CTCCGACTTTCCCATCCAAAACCTGCCCTATGGTGTTTTCTCCACTCAAAGCAACCCAAA
GCCACGGATTGGTGTAGCCATCGGTGACCAGATCTTGGACCTGAGTGTCATTAAACACCT
CTTTACCGGACCTGCCCTTTCCAAACATCAACATGTCTTCGATGAGACAACTCTCAATAA
CTTCATGGGTCTGGGTCAAGCTGCATGGAAGGAGGCAAGAGCATCCTTACAGAACTTACT
GTCTGCCAGCCAAGCCCGGCTCAGAGATGACAAGGAGCTTCGGCAGCGTGCATTCACCTC
CCAGGCTTCTGCGACAATGCACCTTCCTGCTACCATAGGAGACTACACGGACTTCTACTC
TTCTCGGCAGCATGCCACCAATGTTGGCATTATGTTCAGAGGCAAGGAGAATGCGCTGTT
GCCAAATTGGCTCCACTTACCTGTGGGATACCATGGCCGAGCTTCCTCCATTGTGGTATC
TGGAACCCCGATTCGAAGACCCATGGGGCAGATGAGACCTGATAACTCAAAGCCTCCTGT
GTATGGTGCCTGCAGACTCTTAGACATGGAGTTGGAAATGGCTTTCTTCGTAGGCCCTGG
GAACAGATTCGGAGAGCCAATCCCCATTTCCAAAGCCCATGAACACATTTTCGGGATGGT
CCTCATGAACGACTGGAGCGCACGAGACATCCAGCAATGGGAGTACGTCCCACTTGGGCC
ATTCCTGGGGAAAAGCTTTGGAACCACAATCTCCCCGTGGGTGGTGCCTATGGATGCCCT
CATGCCCTTTGTGGTGCCAAACCCAAAGCAGGACCCCAAGCCCTTGCCATATCTCTGCCA
CAGCCAGCCCTACACATTTGATATCAACCTGTCTGTCTCTTTGAAAGGAGAAGGAATGAG
CCAGGCGGCTACCATCTGCAGGTCTAACTTTAAGCACATGTACTGGACCATGCTGCAGCA
ACTCACACACCACTCTGTTAATGGATGCAACCTGAGACCTGGGGACCTCTTGGCTTCTGG
AACCATCAGTGGATCAGACCCTGAAAGCTTTGGCTCCATGCTGGAACTGTCCTGGAAGGG
AACAAAGGCCATCGATGTGGGGCAGGGGCAGACCAGGACCTTCCTGCTGGACGGCGATGA
AGTCATCATAACAGGTCACTGCCAGGGGGACGGCTACCGTGTTGGCTTTGGCCAGTGTGC
TGGGAAAGTGCTGCCTGCCCTTTCACCAGCCTAAACACATCACAACCACAACCTTCTCAG
GTAACTATACTTGGGACTTAAAAAACATAATCATAATCATTTTTCCTAAAACGATCAAGA
CTGATAACCATTTGACAAGAGCCATACAGACAAGCACCAGCTGGCACTCTTAGGTCTTCA
CGTATGGTCATCAGTTTGGGTTCCATTTGTAGATAAGAAACTGAACATATAAAGGTCTAG
GTTAATGCAATTTACACAAAAGGAGACCAAACCAGGGAGAGAAGGAACCAAAATTAAAAA
TTCAAACCAGAGCAAAGGAGTTAGCCCTGGTTTTGCTCTGACTTACATGAACCACTATGT
GGAGTCCTCCATGTTAGCCTAGTCAAGCTTATCCTCTGGATGAAGTTGAAACCATATGAA
GGAATATTTGGGGGGTGGGTCAAAACAGTTGTGTATCAATGATTCCATGTGGTTTGACCC
AATCATTCTGTGAATCCATTTCAACAGAAGATACAACGGGTTCTGTTTCATAATAAGTGA
TCCACTTCCAAATTTCTGATGTGCCCCATGCTAAGCTTTAACAGAATTTATCTTCTTATG
ACAAAGCAGCCTCCTTTGAAAATATAGCCAACTGCACACAGCTATGTTGATCAATTTTGT
TTATAATCTTGCAGAAGAGAATTTTTTAAAATAGGGCAATAATGGAAGGCTTTGGCAAAA
AAATTGTTTCTCCATATGAAAACAAAAAACTTATTTTTTTATTCAAGCAAAGAACCTATA
GACATAAGGCTATTTCAAAATTATTTCAGTTTTAGAAAGAATTGAAAGTTTTGTAGCATT
CTGAGAAGACAGCTTTCATTTGTAATCATAGGTAATATGTAGGTCCTCAGAAATGGTGAG
ACCCCTGACTTTGACACTTGGGGACTCTGAGGGACCAGTGATGAAGAGGGCACAACTTAT
ATCACACATGCACGAGTTGGGGTGAGAGGGTGTCACAACATCTATCAGTGTGTCATCTGC
CCACCAAGTAACAGATGTCAGCTAAGACTAGGTCATGTGTAGGCTGTCTACACCAGTGAA
AATCGCAAAAAGAATCTAAGAAATTCCACATTTCTAGAAAATAGGTTTGGAAACCGTATT
CCATTTTACAAAGGACACTTACATTTCTCTTTTTGTTTTCCAGGCTACCCTGAGAAAAAA
AGACATGAAGACTCAGGACTCATCTTTTCTGTTGGTGTAAAATCAACACCCTAAGGAACA
CAAATTTCTTTAAACATTTGACTTCTTGTCTCTGTGCTGCAATTAATAAAAAATGGAAAG
AATCTACTCTGTGGTTCAGAACTCTATCTTCCAAAGGCGCGCTTCACCCTAGCAGCCTCT
TTGGCTCAGAGGAATCCCTGCCTTTCCTCCCTTCATCTCAGCAGAGAATGTAGTTCCACA
TGGGCAACACAATGAAAATAAACGTTAATACTCTCCCATCTTATGGGTGGTGACCCTAGA
AACCAATACTTCAACATTACGAGAATTCTGAATGAGAGACTAAAAGCTTATGAACTGTGG
CTTTCCTTTGTCAGTGGGACTCTAAGAATGAGTTGGGGACAAAAGAGATAGGAATGGCTT
TAAAGGTGACTAGTTGAACTGATAAAGTAAATGAACTGAGGAAAAAAAATATCACTCAAC
ctgcaggGGACGTCCTACGTAATGCATaggaacccctagtgatggagttggccactccct
ctctgcgcgctcgctcgctcactgaggccgggcgaccaaaggtcgcccgacgcccgggct
ttgcccgggcggcctcagtgagcgagcgagcgcgcagagagggagtggccaagaattaat
tccgtgtattctatagtgtcacctaaatcgtatgtgtatgatacataaggttatgtatta
attgtagccgcgttctaacgacaatatgtacaagcctaattgtgtagcatctggcttact
gaagcagaccctatcatctctctcgtaaactgccgtcagagtcggtttggttggacgaac
cttctgagtttctggtaacgccgtcccgcacccggaaatggtcagcgaaccaatcagcag
ggtcatcgctagc
SEQ ID NO. 2: AAV-DJ-mha-hFAH (PM-0549)
cagatcctctacgccggacgcatcgtggccggcatcaccggcgccacaggtgcggttgct
ggcgcctatatcgccgacatcaccgatggggaagatcgggctcgccacttcgggctcatg
agcgcttgtttcggcgtgggtatggtggcaggccccgtggccgggggactgttgggcgcc
atctccttgcatgcaccattccttgcggcggcggtgctcaacggcctcaacctactactg
ggctgcttcctaatgcaggagtcgcataagggagagcgtcgaatggtgcactctcagtac
aatctgctctgatgccgcatagttaagccagccccgacacccgccaacacccgctgacgc
gccctgacgggcttgtctgctcccggcatccgcttacagacaagctgtgaccgtctccgg
gagctgcatgtgtcagaggttttcaccgtcatcaccgaaacgcgcgagacgaaagggcct
cgtgatacgcctatttttataggttaatgtcatgataataatggtttcttagacgtcagg
tggcacttttcggggaaatgtgcgcggaacccctatttgtttatttttctaaatacattc
aaatatgtatccgctcatgagacaataaccctgataaatgcttcaataatattgaaaaag
gaagagtatgagccatattcaacgggaaacgtcgaggccgcgattaaattccaacatgga
tgctgatttatatgggtataaatgggctcgcgataatgtcgggcaatcaggtgcgacaat
ctatcgcttgtatgggaagcccgatgcgccagagttgtttctgaaacatggcaaaggtag
cgttgccaatgatgttacagatgagatggtcagactaaactggctgacggaatttatgcc
tcttccgaccatcaagcattttatccgtactcctgatgatgcatggttactcaccactgc
gatccccggaaaaacagcattccaggtattagaagaatatcctgattcaggtgaaaatat
tgttgatgcgctggcagtgttcctgcgccggttgcattcgattcctgtttgtaattgtcc
ttttaacagcgatcgcgtatttcgtctcgctcaggcgcaatcacgaatgaataacggttt
ggttgatgcgagtgattttgatgacgagcgtaatggctggcctgttgaacaagtctggaa
agaaatgcataaacttttgccattctcaccggattcagtcgtcactcatggtgatttctc
acttgataaccttatttttgacgaggggaaattaataggttgtattgatgttggacgagt
cggaatcgcagaccgataccaggatcttgccatcctatggaactgcctcggtgagttttc
tccttcattacagaaacggctttttcaaaaatatggtattgataatcctgatatgaataa
attgcagtttcatttgatgctcgatgagtttttctaactgtcagaccaagtttactcata
tatactttagattgatttaaaacttcatttttaatttaaaaggatctaggtgaagatcct
ttttgataatctcatgaccaaaatcccttaacgtgagttttcgttccactgagcgtcaga
ccccgtagaaaagatcaaaggatcttcttgagatcctttttttctgcgcgtaatctgctg
cttgcaaacaaaaaaaccaccgctaccagcggtggtttgtttgccggatcaagagctacc
aactctttttccgaaggtaactggcttcagcagagcgcagataccaaatactgttcttct
agtgtagccgtagttaggccaccacttcaagaactctgtagcaccgcctacatacctcgc
tctgctaatcctgttaccagtggctgctgccagtggcgataagtcgtgtcttaccgggtt
ggactcaagacgatagttaccggataaggcgcagcggtcgggctgaacggggggttcgtg
cacacagcccagcttggagcgaacgacctacaccgaactgagatacctacagcgtgagct
atgagaaagcgccacgcttcccgaagggagaaaggcggacaggtatccggtaagcggcag
ggtcggaacaggagagcgcacgagggagcttccagggggaaacgcctggtatctttatag
tcctgtcgggtttcgccacctctgacttgagcgtcgatttttgtgatgctcgtcaggggg
gcggagcctatggaaaaacgccagcaacgcggcctttttacggttcctggccttttgctg
gccttttgctcacatgttctttcctgcgttatcccctgattctgtggataaccgtattac
cgcctttgagtgagctgataccgctcgccgcagccgaacgaccgagcgcagcgagtcagt
gagcgaggaagcggaagagcgcccaatacgcaaaccgcctctccccgcgcgttggccgat
tcattaatgcagctgtggaatgtgtgtcagttagggtgtggaaagtccccaggctcccca
gcaggcagaagtatgcaaagcatgcatctcaattagtcagcaaccaggtgtggaaagtcc
ccaggctccccagcaggcagaagtatgcaaagcatgcatctcaattagtcagcaaccata
gtcccgcccctaactccgcccatcccgcccctaactccgcccagttccgcccattctccg
ccccatggctgactaattttttttatttatgcagaggccgaggccgcctcggcctctgag
ctattccagaagtagtgaggaggcttttttggaggcctaggcttttgcaaaaagttggcc
actccctctctgcgcgctcgctcgctcactgaggccgcccgggcaaagcccgggcgtcgg
gcgacctttggtcgcccggcctcagtgagcgagcgagcgcgcagagagggagtggccaac
tccatcactaggggttcctTACGTAATGCATGGATCCCCTAGGgcggccgcCTGAAACTA
GACAAAACCCGTGTGACTGGCATCGATTATTCTATTTGATCTAGCTAGTCCTAGCAAAGT
GACAACTGCTACTCCCCTCCTACACAGCCAAGATTCCTAAGTTGGCAGTGGCATGCTTAA
TCCTCAAAGCCAAAGTTACTTGGCTCCAAGATTTATAGCCTTAAACTGTGGCCTCACATT
CCTTCCTATCTTACTTTCCTGCACTGGGGTAAATGTCTCCTTGCTCTTCTTGCTTTCTGT
CCTACTGCAGGGCTCTTGCTGAGCTGGTGAAGCACAAGCCCAAGGCTACAGCGGAGCAAC
TGAAGACTGTCATGGATGACTTTGCACAGTTCCTGGATACATGTTGCAAGGCTGCTGACA
AGGACACCTGCTTCTCGACTGAGGTCAGAAACGTTTTTGCATTTTGACGATGTTCAGTTT
CCATTTTCTGTGCACGTGGTCAGGTGTAGCTCTCTGGAACTCACACACTGAATAACTCCA
CCAATCTAGATGTTGTTCTCTACGTAACTGTAATAGAAACTGACTTACGTAGCTTTTAAT
TTTTATTTTCTGCCACACTGCTGCCTATTAAATACCTATTATCACTATTTGGTTTCAAAT
TTGTGACACAGAAGAGCATAGTTAGAAATACTTGCAAAGCCTAGAATCATGAACTCATTT
AAACCTTGCCCTGAAATGTTTCTTTTTGAATTGAGTTATTTTACACATGAATGGACAGTT
ACCATTATATATCTGAATCATTTCACATTCCCTCCCATGGCCTAACAACAGTTTATCTTC
TTATTTTGGGCACAACAGATGTCAGAGAGCCTGCTTTAGGAATTCTAAGTAGAACTGTAA
TTAAGCAATGCAAGGCACGTACGTTTACTATGTCATTGCCTATGGCTATGAAGTGCAAAT
CCTAACAGTCCTGCTAATACTTTTCTAACATCCATCATTTCTTTGTTTTCAGGGTCCAAA
CCTTGTCACTAGATGCAAAGACGCCTTAGCCggaagcggcgccaccaatttcagcctgct
gaaacaggccggcgacgtggaagagaaccctggcccttccttcatcccggtggccgagga
ttccgacttccccatccacaacctgccctacggcgtcttctcgaccagaggcgacccaag
accgaggataggtgtggccattggcgaccagatcctggacctcagcatcatcaagcacct
ctttactggtcctgtcctctccaaacaccaggatgtcttcaatcagcctacactcaacag
cttcatgggcctgggtcaggctgcctggaaggaggcgagagtgttcttgcagaacttgct
gtctgtgagccaagccaggctcagagatgacaccgaacttcggaagtgtgcattcatctc
ccaggcttctgccacgatgcaccttccagccaccataggagactacacagacttctattc
ctctcggcagcatgctaccaacgtcggaatcatgttcagggacaaggagaatgcgttgat
gccaaattggctgcacttaccagtgggctaccatggccgtgcctcctctgtcgtggtgtc
tggcaccccaatccgaaggcccatgggacagatgaaacctgatgactctaagcctcccgt
atatggtgcctgcaagctcttggacatggagctggaaatggctttttttgtaggccctgg
aaacagattgggagagccgatccccatttccaaggcccatgagcacatttttggaatggt
ccttatgaacgactggagtgcacgagacattcagaagtgggagtatgtccctctcgggcc
attccttgggaagagttttgggaccactgtctctccgtgggtggtgcccatggatgctct
catgccctttgctgtgcccaacccgaagcaggaccccaggcccctgccgtatctgtgcca
tgacgagccctacacatttgacatcaacctctctgttaacctgaaaggagaaggaatgag
ccaggcggctaccatatgcaagtccaattttaagtacatgtactggacgatgctgcagca
gctcactcaccactctgtcaacggctgcaacctgcggccgggggacctcctggcttctgg
gaccatcagcgggccggagccagaaaacttcggctccatgttggaactgtcgtggaaggg
aacgaagcccatagacctggggaatggtcagaccaggaagtttctgctggacggggatga
agtcatcataacagggtactgccagggggatggttaccgcatcggctttggccagtgtgc
tggaaaagtgctgcctgctctcctgccatcaTAAACACATCACAACCACAACCTTCTCAG
GTAACTATACTTGGGACTTAAAAAACATAATCATAATCATTTTTCCTAAAACGATCAAGA
CTGATAACCATTTGACAAGAGCCATACAGACAAGCACCAGCTGGCACTCTTAGGTCTTCA
CGTATGGTCATCAGTTTGGGTTCCATTTGTAGATAAGAAACTGAACATATAAAGGTCTAG
GTTAATGCAATTTACACAAAAGGAGACCAAACCAGGGAGAGAAGGAACCAAAATTAAAAA
TTCAAACCAGAGCAAAGGAGTTAGCCCTGGTTTTGCTCTGACTTACATGAACCACTATGT
GGAGTCCTCCATGTTAGCCTAGTCAAGCTTATCCTCTGGATGAAGTTGAAACCATATGAA
GGAATATTTGGGGGGTGGGTCAAAACAGTTGTGTATCAATGATTCCATGTGGTTTGACCC
AATCATTCTGTGAATCCATTTCAACAGAAGATACAACGGGTTCTGTTTCATAATAAGTGA
TCCACTTCCAAATTTCTGATGTGCCCCATGCTAAGCTTTAACAGAATTTATCTTCTTATG
ACAAAGCAGCCTCCTTTGAAAATATAGCCAACTGCACACAGCTATGTTGATCAATTTTGT
TTATAATCTTGCAGAAGAGAATTTTTTAAAATAGGGCAATAATGGAAGGCTTTGGCAAAA
AAATTGTTTCTCCATATGAAAACAAAAAACTTATTTTTTTATTCAAGCAAAGAACCTATA
GACATAAGGCTATTTCAAAATTATTTCAGTTTTAGAAAGAATTGAAAGTTTTGTAGCATT
CTGAGAAGACAGCTTTCATTTGTAATCATAGGTAATATGTAGGTCCTCAGAAATGGTGAG
ACCCCTGACTTTGACACTTGGGGACTCTGAGGGACCAGTGATGAAGAGGGCACAACTTAT
ATCACACATGCACGAGTTGGGGTGAGAGGGTGTCACAACATCTATCAGTGTGTCATCTGC
CCACCAAGTAACAGATGTCAGCTAAGACTAGGTCATGTGTAGGCTGTCTACACCAGTGAA
AATCGCAAAAAGAATCTAAGAAATTCCACATTTCTAGAAAATAGGTTTGGAAACCGTATT
CCATTTTACAAAGGACACTTACATTTCTCTTTTTGTTTTCCAGGCTACCCTGAGAAAAAA
AGACATGAAGACTCAGGACTCATCTTTTCTGTTGGTGTAAAATCAACACCCTAAGGAACA
CAAATTTCTTTAAACATTTGACTTCTTGTCTCTGTGCTGCAATTAATAAAAAATGGAAAG
AATCTACTCTGTGGTTCAGAACTCTATCTTCCAAAGGCGCGCTTCACCCTAGCAGCCTCT
TTGGCTCAGAGGAATCCCTGCCTTTCCTCCCTTCATCTCAGCAGAGAATGTAGTTCCACA
TGGGCAACACAATGAAAATAAACGTTAATACTCTCCCATCTTATGGGTGGTGACCCTAGA
AACCAATACTTCAACATTACGAGAATTCTGAATGAGAGACTAAAAGCTTATGAACTGTGG
CTTTCCTTTGTCAGTGGGACTCTAAGAATGAGTTGGGGACAAAAGAGATAGGAATGGCTT
TAAAGGTGACTAGTTGAACTGATAAAGTAAATGAACTGAGGAAAAAAAATATCACTCAAc
ctgcaggGGACGTCCTACGTAATGCATaggaacccctagtgatggagttggccactccct
ctctgcgcgctcgctcgctcactgaggccgggcgaccaaaggtcgcccgacgcccgggct
ttgcccgggcggcctcagtgagcgagcgagcgcgcagagagggagtggccaagaattaat
tccgtgtattctatagtgtcacctaaatcgtatgtgtatgatacataaggttatgtatta
attgtagccgcgttctaacgacaatatgtacaagcctaattgtgtagcatctggcttact
gaagcagaccctatcatctctctcgtaaactgccgtcagagtcggtttggttggacgaac
cttctgagtttctggtaacgccgtcccgcacccggaaatggtcagcgaaccaatcagcag
ggtcatcgctagc
SEQ ID NO. 3: AAV-DJ-mha-mFAH (PM-0549)
gtatatatgagtaaacttggtctgacagttaccaatgcttaatcagtgaggcacctatct
cagcgatctgtctatttcgttcatccatagttgcctgactccccgtcgtgtagataacta
cgatacgggagggcttaccatctggccccagtgctgcaatgataccgcgagacccacgct
caccggctccagatttatcagcaataaaccagccagccggaagggccgagcgcagaagtg
gtcctgcaactttatccgcctccatccagtctattaattgttgccgggaagctagagtaa
gtagttcgccagttaatagtttgcgcaacgttgttgccattgctacaggcatcgtggtgt
cacgctcgtcgtttggtatggcttcattcagctccggttcccaacgatcaaggcgagtta
catgatcccccatgttgtgcaaaaaagcggttagctccttcggtcctccgatcgttgtca
gaagtaagttggccgcagtgttatcactcatggttatggcagcactgcataattctctta
ctgtcatgccatccgtaagatgcttttctgtgactggtgagtactcaaccaagtcattct
gagaatagtgtatgcggcgaccgagttgctcttgcccggcgtcaatacgggataataccg
cgccacatagcagaactttaaaagtgctcatcattggaaaacgttcttcggggcgaaaac
tctcaaggatcttaccgctgttgagatccagttcgatgtaacccactcgtgcacccaact
gatcttcagcatcttttactttcaccagcgtttctgggtgagcaaaaacaggaaggcaaa
atgccgcaaaaaagggaataagggcgacacggaaatgttgaatactcatactcttccttt
ttcaatattattgaagcatttatcagggttattgtctcatgagcggatacatatttgaat
gtatttagaaaaataaacaaataggggttccgcgcacatttccccgaaaagtgccacctg
acgtctaagaaaccattattatcatgacattaacctataaaaataggcgtatcacgaggc
cctttcgtctcgcgcgtttcggtgatgacggtgaaaacctctgacacatgcagctcccgg
agacggtcacagcttgtctgtaagcggatgccgggagcagacaagcccgtcagggcgcgt
cagcgggtgttggcgggtgtcggggctggcttaactatgcggcatcagagcagattgtac
tgagagtgcaccattcgacgctctcccttatgcgactcctgcattaggaagcagcccagt
agtaggttgaggccgttgagcaccgccgccgcaaggaatggtgcatgcaaggagatggcg
cccaacagtcccccggccacggggcctgccaccatacccacgccgaaacaagcgctcatg
agcccgaagtggcgagcccgatcttccccatcggtgatgtcggcgatataggcgccagca
accgcacctgtggcgccggtgggtcaccaagcaggaagtcaaagactttttccggtgggc
aaaggatcacgtggttgaggtggagcatgaattctacgtcaaaaagggtggagccaagaa
aagacccgcccccagtgacgcagatataagtgagcccaaacgggtgcgcgagtcagttgc
gcagccatcgacgtcagacgcggaagcttcgatcaactacgcagacaggtaccaaaacaa
atgttctcgtcacgtgggcatgaatctgatgctgtttccctgcagacaatgcgagagaat
gaatcagaattcaaatatctgcttcactcacggacagaaagactgtttagagtgctttcc
cgtgtcagaatctcaacccgtttctgtcgtcaaaaaggcgtatcagaaactgtgctacat
tcatcatatcatgggaaaggtgccagacgcttgcactgcctgcgatctggtcaatgtgga
tttggatgactgcatctttgaacaataaatgATTTAAATCAGGTatggctgccgatggtt
atcttccagattggctcgaggacactctctctgaaggaataagacagtggtggaagctca
aacctggcccaccaccaccaaagcccgcagagcggcataaggacgacagcaggggtcttg
tgcttcctgggtacaagtacctcggacccttcaacggactcgacaagggagagccggtca
acgaggcagacgccgcggccctcgagcacgacaaagcctacgaccggcagctcgacagcg
gagacaacccgtacctcaagtacaaccacgccgacgccgagttccaggagcggctcaaag
aagatacgtcttttgggggcaacctcgggcgagcagtcttccaggccaaaaagaggcttc
ttgaacctcttggtctggttgaggaagcggctaagacggctcctggaaagaagaggcctg
tagagcactctcctgtggagccagactcctcctcgggaaccggaaaggcgggccagcagc
ctgcaagaaaaagattgaattttggtcagactggagacgcagactcagtcccagaccctc
aaccaatcggagaacctcccgcagccccctcaggtgtgggatctcttacaatggctgcag
gcggtggcgcaccaatggcagacaataacgagggcgccgacggagtgggtaattcctcgg
gaaattggcattgcgattccacatggatgggcgacagagtcatcaccaccagcacccgaa
cctgggccctgcccacctacaacaaccacctctacaagcaaatctccaacagcacatctg
gaggatcttcaaatgacaacgcctacttcggctacagcaccccctgggggtattttgact
ttaacagattccactgccacttttcaccacgtgactggcagcgactcatcaacaacaact
ggggattccggcccaagagactcagcttcaagctcttcaacatccaggtcaaggaggtca
cgcagaatgaaggcaccaagaccatcgccaataacctcaccagcaccatccaggtgttta
cggactcggagtaccagctgccgtacgttctcggctctgcccaccagggctgcctgcctc
cgttcccggcggacgtgttcatgattccccagtacggctacctaacactcaacaacggta
gtcaggccgtgggacgctcctccttctactgcctggaatactttccttcgcagatgctga
gaaccggcaacaacttccagtttacttacaccttcgaggacgtgcctttccacagcagct
acgcccacagccagagcttggaccggctgatgaatcctctgattgaccagtacctgtact
acttgtctcggactcaaacaacaggaggcacgacaaatacgcagactctgggcttcagcc
aaggtgggcctaatacaatggccaatcaggcaaagaactggctgccaggaccctgttacc
gccagcagcgagtatcaaagacatctgcggataacaacaacagtgaatactcgtggactg
gagctaccaagtaccacctcaatggcagagactctctggtgaatccgggcccggccatgg
caagccacaaggacgatgaagaaaagttttttcctcagagcggggttctcatctttggga
agcaaggctcagagaaaacaaatgtggacattgaaaaggtcatgattacagacgaagagg
aaatcaggacaaccaatcccgtggctacggagcagtatggttctgtatctaccaacctcc
agagaggcaacagacaagcagctaccgcagatgtcaacacacaaggcgttcttccaggca
tggtctggcaggacagagatgtgtaccttcaggggcccatctgggcaaagattccacaca
cggacggacattttcacccctctcccctcatgggtggattcggacttaaacaccctccgc
ctcagatcctgatcaagaacacgcctgtacctgcggatcctccgaccaccttcaaccagt
caaagctgaactctttcatcacccagtattctactggccaagtcagcgtggagatcgagt
gggagctgcagaaggaaaacagcaagcgctggaaccccgagatccagtacacctccaact
actacaaatctacaagtgtggactttgctgttaatacagaaggcgtgtactctgaacccc
gccccattggcacccgttacctcacccgtaatctgtaaTTGCTTGTTAATCAATAAACCG
TTTAATTCGTTTCAGTTGAACTTTGGTCTCTGCGTATTTCTTTCTTATCTAGTTTCCATA
TGCATGTAGATAAGTAGCATGGCGGGTTAATCATTAACTAAccggtacctctagaactat
agctagcgatgaccctgctgattggttcgctgaccatttccgggtgcgggacggcgttac
cagaaactcagaaggttcgtccaaccaaaccgactctgacggcagtttacgagagagatg
atagggtctgcttcagtaagccagatgctacacaattaggcttgtacatattgtcgttag
aacgcggctacaattaatacataaccttatgtatcatacacatacgatttaggtgacact
atagaatacacggaattaattcttggccactccctctctgcgcgctcgctcgctcactga
ggccgcccgggcaaagcccgggcgtcgggcgacctttggtcgcccggcctcagtgagcga
gcgagcgcgcagagagggagtggccaactccatcactaggggttccttaccgaCTGAAAC
TAGACAAAACCCGTGTGACTGGCATCGATTATTCTATTTGATCTAGCTAGTCCTAGCAAA
GTGACAACTGCTACTCCCCTCCTACACAGCCAAGATTCCTAAGTTGGCAGTGGCATGCTT
AATCCTCAAAGCCAAAGTTACTTGGCTCCAAGATTTATAGCCTTAAACTGTGGCCTCACA
TTCCTTCCTATCTTACTTTCCTGCACTGGGGTAAATGTCTCCTTGCTCTTCTTGCTTTCT
GTCCTACTGCAGGGCTCTTGCTGAGCTGGTGAAGCACAAGCCCAAGGCTACAGCGGAGCA
ACTGAAGACTGTCATGGATGACTTTGCACAGTTCCTGGATACATGTTGCAAGGCTGCTGA
CAAGGACACCTGCTTCTCGACTGAGGTCAGAAACGTTTTTGCATTTTGACGATGTTCAGT
TTCCATTTTCTGTGCACGTGGTCAGGTGTAGCTCTCTGGAACTCACACACTGAATAACTC
CACCAATCTAGATGTTGTTCTCTACGTAACTGTAATAGAAACTGACTTACGTAGCTTTTA
ATTTTTATTTTCTGCCACACTGCTGCCTATTAAATACCTATTATCACTATTTGGTTTCAA
ATTTGTGACACAGAAGAGCATAGTTAGAAATACTTGCAAAGCCTAGAATCATGAACTCAT
TTAAACCTTGCCCTGAAATGTTTCTTTTTGAATTGAGTTATTTTACACATGAATGGACAG
TTACCATTATATATCTGAATCATTTCACATTCCCTCCCATGGCCTAACAACAGTTTATCT
TCTTATTTTGGGCACAACAGATGTCAGAGAGCCTGCTTTAGGAATTCTAAGTAGAACTGT
AATTAAGCAATGCAAGGCACGTACGTTTACTATGTCATTGCCTATGGCTATGAAGTGCAA
ATCCTAACAGTCCTGCTAATACTTTTCTAACATCCATCATTTCTTTGTTTTCAGGGTCCA
AACCTTGTCACTAGATGCAAAGACGCCTTAGCCggaagcggcgccaccaatttcagcctg
ctgaaacaggccggcgacgtggaagagaaccctggccctTCCTTTATTCCAGTGGCCGAG
GACTCCGACTTTCCCATCCAAAACCTGCCCTATGGTGTTTTCTCCACTCAAAGCAACCCA
AAGCCACGGATTGGTGTAGCCATCGGTGACCAGATCTTGGACCTGAGTGTCATTAAACAC
CTCTTTACCGGACCTGCCCTTTCCAAACATCAACATGTCTTCGATGAGACAACTCTCAAT
AACTTCATGGGTCTGGGTCAAGCTGCATGGAAGGAGGCAAGAGCATCCTTACAGAACTTA
CTGTCTGCCAGCCAAGCCCGGCTCAGAGATGACAAGGAGCTTCGGCAGCGTGCATTCACC
TCCCAGGCTTCTGCGACAATGCACCTTCCTGCTACCATAGGAGACTACACGGACTTCTAC
TCTTCTCGGCAGCATGCCACCAATGTTGGCATTATGTTCAGAGGCAAGGAGAATGCGCTG
TTGCCAAATTGGCTCCACTTACCTGTGGGATACCATGGCCGAGCTTCCTCCATTGTGGTA
TCTGGAACCCCGATTCGAAGACCCATGGGGCAGATGAGACCTGATAACTCAAAGCCTCCT
GTGTATGGTGCCTGCAGACTCTTAGACATGGAGTTGGAAATGGCTTTCTTCGTAGGCCCT
GGGAACAGATTCGGAGAGCCAATCCCCATTTCCAAAGCCCATGAACACATTTTCGGGATG
GTCCTCATGAACGACTGGAGCGCACGAGACATCCAGCAATGGGAGTACGTCCCACTTGGG
CCATTCCTGGGGAAAAGCTTTGGAACCACAATCTCCCCGTGGGTGGTGCCTATGGATGCC
CTCATGCCCTTTGTGGTGCCAAACCCAAAGCAGGACCCCAAGCCCTTGCCATATCTCTGC
CACAGCCAGCCCTACACATTTGATATCAACCTGTCTGTCTCTTTGAAAGGAGAAGGAATG
AGCCAGGCGGCTACCATCTGCAGGTCTAACTTTAAGCACATGTACTGGACCATGCTGCAG
CAACTCACACACCACTCTGTTAATGGATGCAACCTGAGACCTGGGGACCTCTTGGCTTCT
GGAACCATCAGTGGATCAGACCCTGAAAGCTTTGGCTCCATGCTGGAACTGTCCTGGAAG
GGAACAAAGGCCATCGATGTGGGGCAGGGGCAGACCAGGACCTTCCTGCTGGACGGCGAT
GAAGTCATCATAACAGGTCACTGCCAGGGGGACGGCTACCGTGTTGGCTTTGGCCAGTGT
GCTGGGAAAGTGCTGCCTGCCCTTTCACCAGCCTAAACACATCACAACCACAACCTTCTC
AGGTAACTATACTTGGGACTTAAAAAACATAATCATAATCATTTTTCCTAAAACGATCAA
GACTGATAACCATTTGACAAGAGCCATACAGACAAGCACCAGCTGGCACTCTTAGGTCTT
CACGTATGGTCATCAGTTTGGGTTCCATTTGTAGATAAGAAACTGAACATATAAAGGTCT
AGGTTAATGCAATTTACACAAAAGGAGACCAAACCAGGGAGAGAAGGAACCAAAATTAAA
AATTCAAACCAGAGCAAAGGAGTTAGCCCTGGTTTTGCTCTGACTTACATGAACCACTAT
GTGGAGTCCTCCATGTTAGCCTAGTCAAGCTTATCCTCTGGATGAAGTTGAAACCATATG
AAGGAATATTTGGGGGGTGGGTCAAAACAGTTGTGTATCAATGATTCCATGTGGTTTGAC
CCAATCATTCTGTGAATCCATTTCAACAGAAGATACAACGGGTTCTGTTTCATAATAAGT
GATCCACTTCCAAATTTCTGATGTGCCCCATGCTAAGCTTTAACAGAATTTATCTTCTTA
TGACAAAGCAGCCTCCTTTGAAAATATAGCCAACTGCACACAGCTATGTTGATCAATTTT
GTTTATAATCTTGCAGAAGAGAATTTTTTAAAATAGGGCAATAATGGAAGGCTTTGGCAA
AAAAATTGTTTCTCCATATGAAAACAAAAAACTTATTTTTTTATTCAAGCAAAGAACCTA
TAGACATAAGGCTATTTCAAAATTATTTCAGTTTTAGAAAGAATTGAAAGTTTTGTAGCA
TTCTGAGAAGACAGCTTTCATTTGTAATCATAGGTAATATGTAGGTCCTCAGAAATGGTG
AGACCCCTGACTTTGACACTTGGGGACTCTGAGGGACCAGTGATGAAGAGGGCACAACTT
ATATCACACATGCACGAGTTGGGGTGAGAGGGTGTCACAACATCTATCAGTGTGTCATCT
GCCCACCAAGTAACAGATGTCAGCTAAGACTAGGTCATGTGTAGGCTGTCTACACCAGTG
AAAATCGCAAAAAGAATCTAAGAAATTCCACATTTCTAGAAAATAGGTTTGGAAACCGTA
TTCCATTTTACAAAGGACACTTACATTTCTCTTTTTGTTTTCCAGGCTACCCTGAGAAAA
AAAGACATGAAGACTCAGGACTCATCTTTTCTGTTGGTGTAAAATCAACACCCTAAGGAA
CACAAATTTCTTTAAACATTTGACTTCTTGTCTCTGTGCTGCAATTAATAAAAAATGGAA
AGAATCTACTCTGTGGTTCAGAACTCTATCTTCCAAAGGCGCGCTTCACCCTAGCAGCCT
CTTTGGCTCAGAGGAATCCCTGCCTTTCCTCCCTTCATCTCAGCAGAGAATGTAGTTCCA
CATGGGCAACACAATGAAAATAAACGTTAATACTCTCCCATCTTATGGGTGGTGACCCTA
GAAACCAATACTTCAACATTACGAGAATTCTGAATGAGAGACTAAAAGCTTATGAACTGT
GGCTTTCCTTTGTCAGTGGGACTCTAAGAATGAGTTGGGGACAAAAGAGATAGGAATGGC
TTTAAAGGTGACTAGTTGAACTGATAAAGTAAATGAACTGAGGAAAAAAAATATCACTCA
Atcggtaaggaacccctagtgatggagttggccactccctctctgcgcgctcgctcgctc
actgaggccgggcgaccaaaggtcgcccgacgcccgggctttgcccgggcggcctcagtg
agcgagcgagcgcgcagagagggagtggccaactttttgcaaaagcctaggcctccaaaa
aagcctcctcactacttctggaatagctcagaggccgaggcggcctcggcctctgcataa
ataaaaaaaattagtcagccatggggcggagaatgggcggaactgggcggagttaggggc
gggatgggcggagttaggggcgggactatggttgctgactaattgagatgcatgctttgc
atacttctgcctgctggggagcctggggactttccacacctggttgctgactaattgaga
tgcatgctttgcatacttctgcctgctggggagcctggggactttccacaccctaactga
cacacattccacagctgcattaatgaatcggccaacgcgcggggagaggcggtttgcgta
ttgggcgctcttccgcttcctcgctcactgactcgctgcgctcggtcgttcggctgcggc
gagcggtatcagctcactcaaaggcggtaatacggttatccacagaatcaggggataacg
caggaaagaacatgtgagcaaaaggccagcaaaaggccaggaaccgtaaaaaggccgcgt
tgctggcgtttttccataggctccgcccccctgacgagcatcacaaaaatcgacgctcaa
gtcagaggtggcgaaacccgacaggactataaagataccaggcgtttccccctggaagct
ccctcgtgcgctctcctgttccgaccctgccgcttaccggatacctgtccgcctttctcc
cttcgggaagcgtggcgctttctcatagctcacgctgtaggtatctcagttcggtgtagg
tcgttcgctccaagctgggctgtgtgcacgaaccccccgttcagcccgaccgctgcgcct
tatccggtaactatcgtcttgagtccaacccggtaagacacgacttatcgccactggcag
cagccactggtaacaggattagcagagcgaggtatgtaggcggtgctacagagttcttga
agtggtggcctaactacggctacactagaagaacagtatttggtatctgcgctctgctga
agccagttaccttcggaaaaagagttggtagctcttgatccggcaaacaaaccaccgctg
gtagcggtggtttttttgtttgcaagcagcagattacgcgcagaaaaaaaggatctcaag
aagatcctttgatcttttctacggggtctgacgctcagtggaacgaaaactcacgttaag
ggattttggtcatgagattatcaaaaaggatcttcacctagatccttttaaattaaaaat
gaagttttaaatcaatctaaa

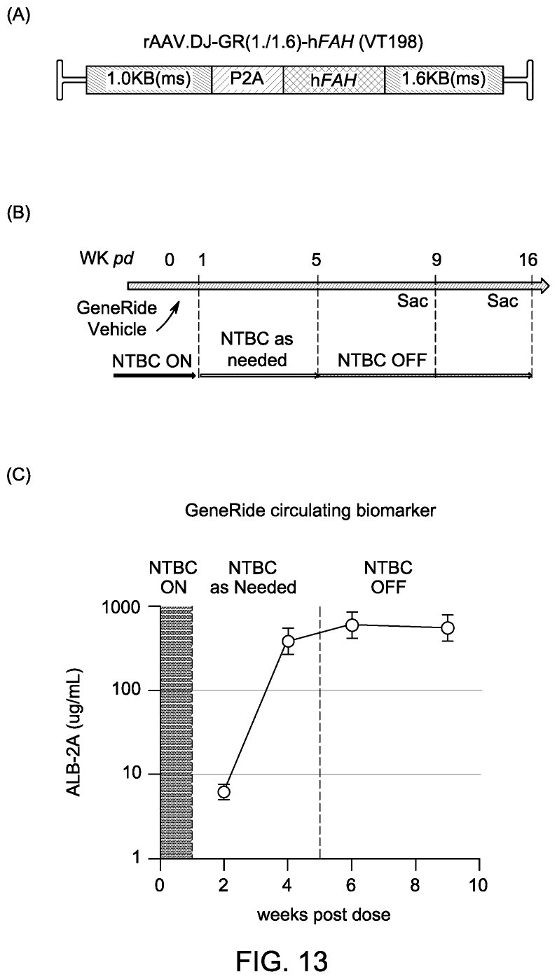

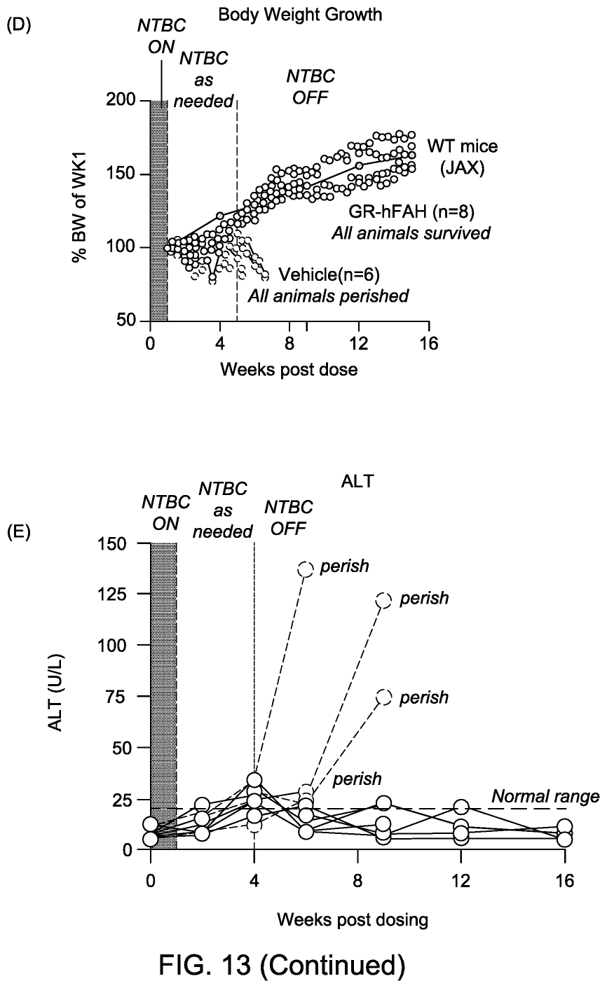

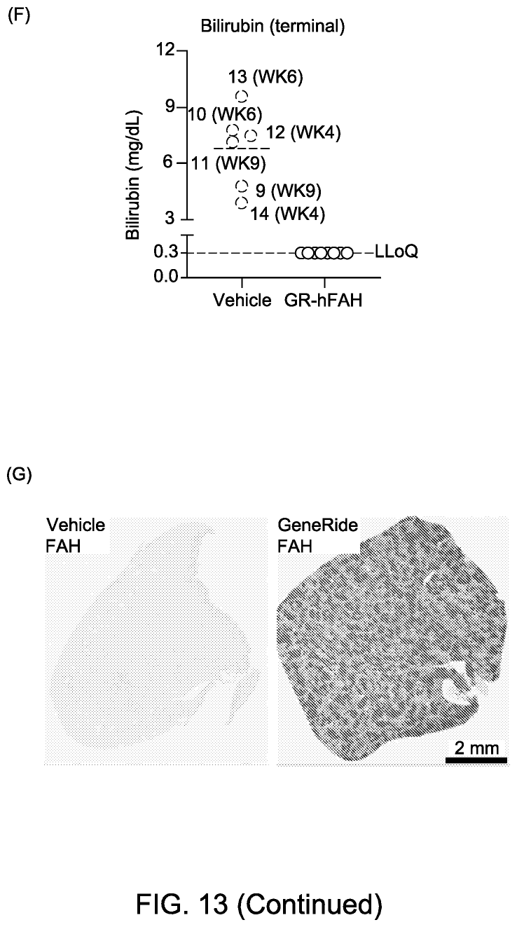

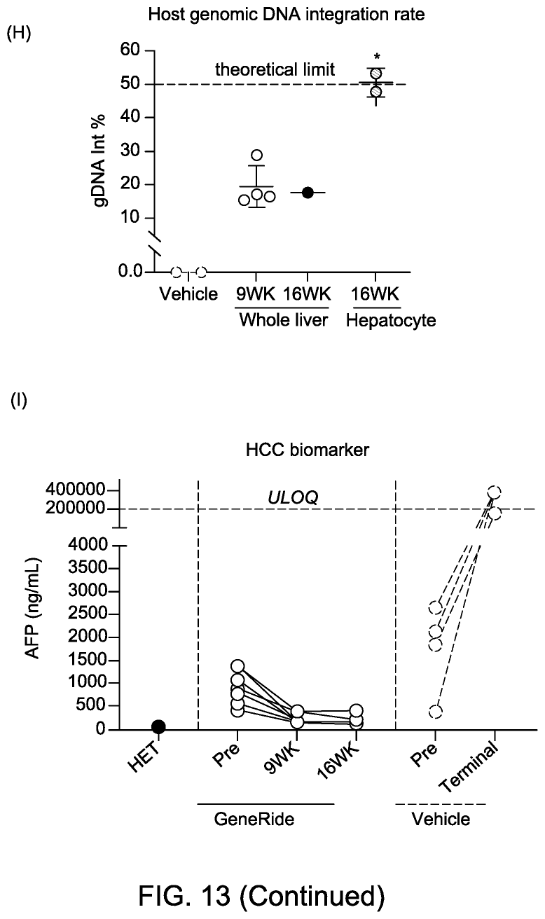

Viral vectors comprising an AAV-DJ viral capsid, human FAH (hFAH) transgene, P2A sequence, a flanking 5′ homology arm 1000 nucleotides (nt) in length, and a 3′ homology arm 1600 nt in length were constructed (FIG. 13A). Homology arms were designed for complementarity to a mouse genomic albumin target integration site. Viral vectors were administered to FRG mice, a mouse model of tyrosinemia, at 4 weeks of age via intravenous injection and maintained on NTBC drinking water (8 mg/L) since birth until 1 week post dosing (FIG. 13B). Between 1 and 4 weeks post dosing, NTBC dosages were adjusted as needed (e.g., if animal body weight dropped 10% below previous week measurement, animal was cycled back to 8 mg/L NTBC drinking water for one week). From 4 weeks post dosing until sacrifice (at 9 or 16 weeks post dosing), NTBC administration was discontinued.

Mice were assessed for circulating GeneRide biomarkers (e.g., levels of ALB-2A) for up to 9 weeks post-treatment (FIG. 13C) and for body growth (through percent body weight change) for up to 16 weeks post-treatment (FIG. 13D). Markers of liver function (e.g., alanine aminotransferase (ALT), bilirubin) were also assessed (FIG. 13E and FIG. 13F). Mice were sacrificed at 9 weeks (9WK; 4 animals harvested) and 16 weeks (16WK; 4 animals harvested) post-treatment and mouse livers were isolated. A subset of treated mice were sacrificed and liver samples were harvested and analyzed through immunohistochemical staining with anti-FAH antibodies (FIG. 13G). Hepatocytes were isolated from a separate subset of treated mice whose livers had been perfused (n=2). Isolated hepatocytes were assessed for gDNA integration (FIG. 13H). Plasma samples from mice were also assessed for presence of alfa-fetoprotein (AFP), a marker for hepatocellular carcinoma (HCC) (FIG. 13I), as compared to healthy Fah+/− heterozygous (HET) mice and vehicle only treated mice.

### Results

Among other things, this example demonstrates that treatment with viral vectors comprising unbalanced homology arms flanking a sequence encoding human FAH (hFAH) may provide improved liver function in a subject suffering from hereditary tyrosinemia 1 (HT1) (e.g., in a FRG mouse model system) as compared to a reference (e.g., untreated or vehicle). In some embodiments, as demonstrated in FIG. 13, panel E and/or panel F, treatment of a subject with viral vectors of the present disclosure may provide reduced levels of biomarkers associated with reduced liver function (e.g., ALT, bilirubin) as compared to a reference (e.g., untreated or vehicle). In some embodiments, as demonstrated in FIG. 13, panel D, treatment of a subject (e.g., a subject suffering from hereditary tyrosinemia) with viral vectors of the present disclosure may allow or restore normal growth (e.g., measured through percentage body weight changes over time) relative to a reference (e.g., untreated or vehicle). In some embodiments, as demonstrated in FIG. 13, panel I, treatment of a subject (e.g., a subject suffering from hereditary tyrosinemia) with viral vectors of the present disclosure may provide reduced levels of a biomarker (e.g., AFP) associated with a disease (e.g., cancer, including HCC). In some embodiments, as demonstrated in FIG. 13, treatment of a subject (e.g., a subject suffering from hereditary tyrosinemia) with viral vectors of the present disclosure may provide one or more of improved liver function (e.g., measured through assessment of markers of liver function), normal growth (e.g., measured through percentage body weight changes over time) relative to a reference (e.g., untreated or vehicle), and reduced levels of a biomarker (e.g., AFP) associated with a disease (e.g., cancer, including HCC).

Among other things, as shown in FIG. 13, panel H, this demonstrates that viral vectors of the present disclosure are capable of integrating a transgene sequence (e.g., FAH) into a genomic target site in a subject. In some embodiments, integration of a transgene sequence (e.g., FAH) may provide a selective advantage for cells (e.g. liver cells) in a subject (e.g., a subject suffering from hereditary tyrosinemia). In some embodiments, treatment of a subject (e.g., a subject suffering from hereditary tyrosinemia) with viral vectors of the present disclosure may enable more than 10% (e.g., 20%, 30%, 40%, 50%) genomic DNA integration of a delivered transgene (e.g., FAH) in cells (e.g., liver cells) within a particular tissue type (e.g. liver). In some embodiments, treatment of a subject (e.g., a subject suffering from hereditary tyrosinemia) with viral vectors of the present disclosure may enable more than 50% (e.g., 60%, 70%, 80%, 90%, 95%, 99%, 100%, etc.) of cells (e.g., liver cells) within a particular tissue type (e.g. liver) to consist of cells that have successfully integrated a delivered transgene (e.g., FAH)

### Example 10: Uneven Homology Arms May Allow for Rapid Selective Expansion of Edited Cells In Vivo

The present example demonstrates that, among other things, viral vectors comprising unbalanced homology arms flanking a sequence encoding fumarylacetoacetate hydrolase (FAH) may be administered to a subject (e.g., a subject suffering from hereditary tyrosinemia type 1) at certain dosages and provide a selective advantage for cells that have successfully integrated a FAH-encoding sequence. Unless otherwise specified, the materials and methods used were as in example 9 above.

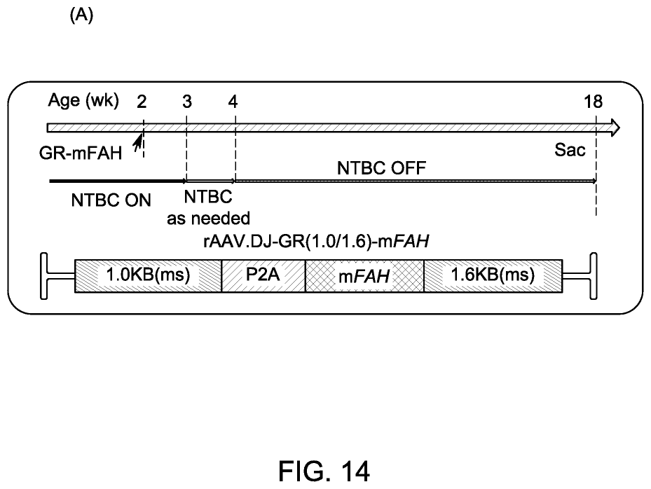

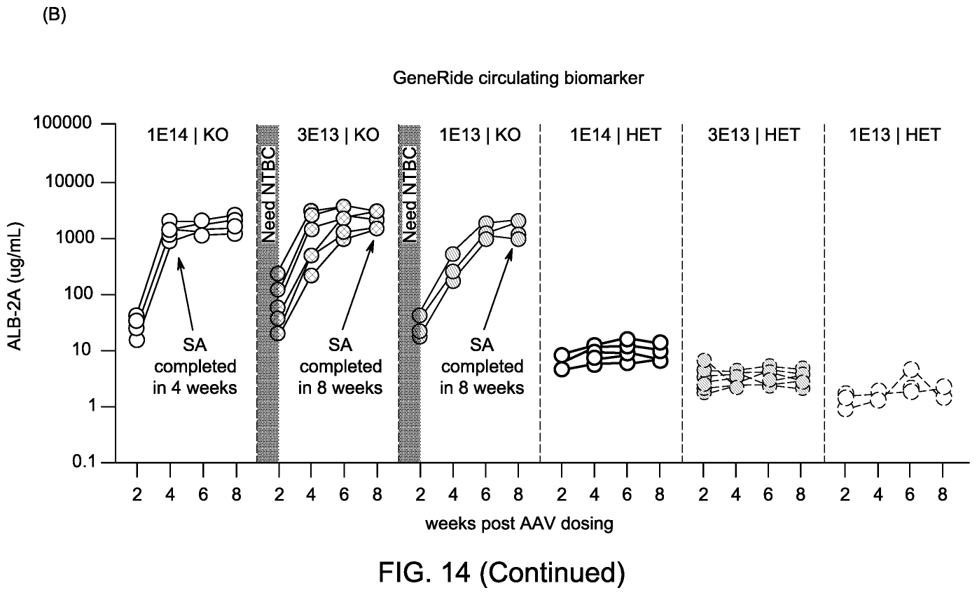

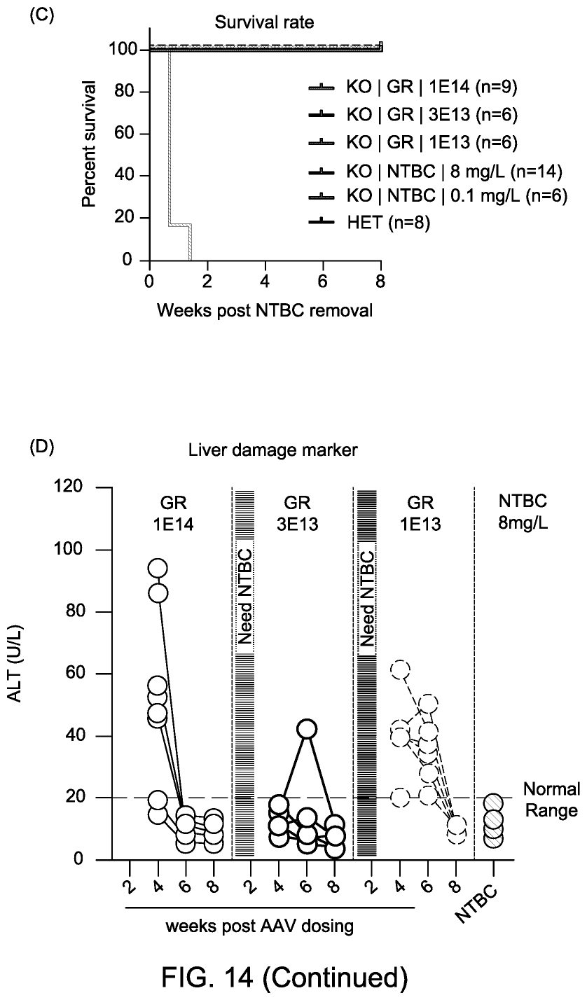

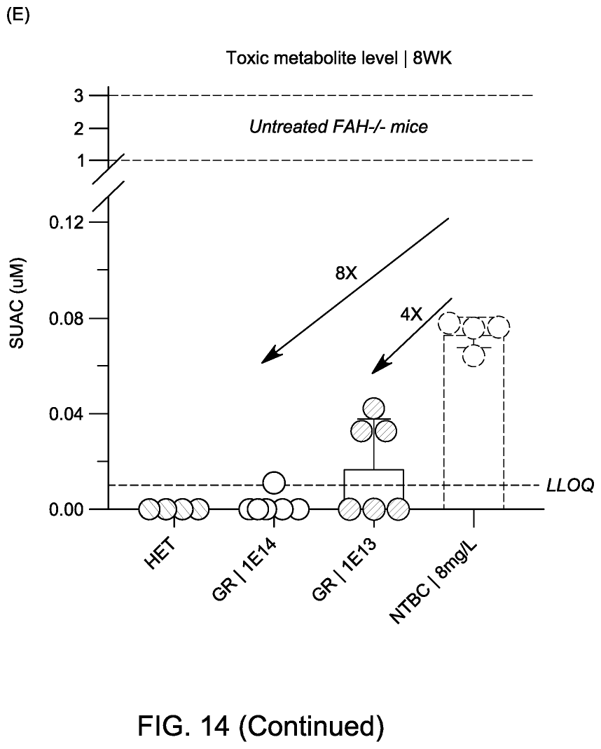

Viral vectors comprising an AAV-DJ viral capsid, mouse FAH (mFAH) transgene, P2A sequence, a flanking 5′ homology arm 1000 nucleotides (nt) in length, and a 3′ homology arm 1600 nt in length were constructed (see FIG. 14, panel A). Homology arms were designed for complementarity to a mouse genomic albumin target integration site. Vectors herein described were administered to Fah−/− mice via intravenous injection at two weeks of age (FIG. 14, panel A). Mice were supplied with 8 mg/L of NTBC from birth until 1 week post administration of GeneRide vectors (3 weeks of age). Between 3 and 4 weeks of age, 8 mg/L NTBC drinking water was only supplied to animals observed with 10% body weight drop from previous week. From 4 weeks of age until sacrifice (at least 18 weeks of age), NTBC administration was discontinued. Another group of Fah−/− mice was always maintained with 8 mg/L NTBC drinking water and served as standard of care control group. Mice were assessed for circulating biomarkers (e.g., levels of ALB-2A) for up to 8 weeks post-treatment (FIG. 14, panel B) and survival rate post NTBC removal (FIG. 14, panel C), markers of liver function (e.g., ALT) and presence of toxic metabolites (e.g., succinylacetone (SUAC)) were assessed (FIG. 14, panel D and FIG. 14, panel E).

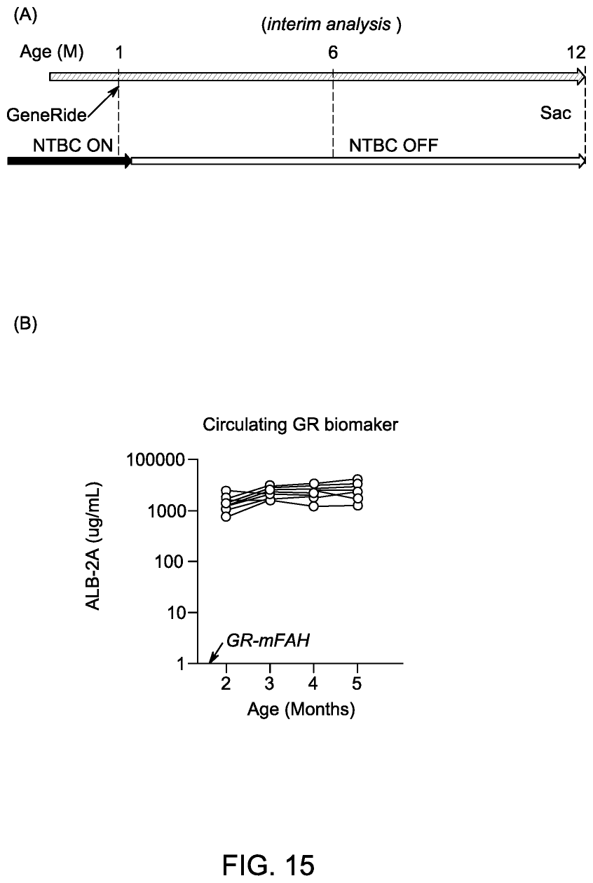

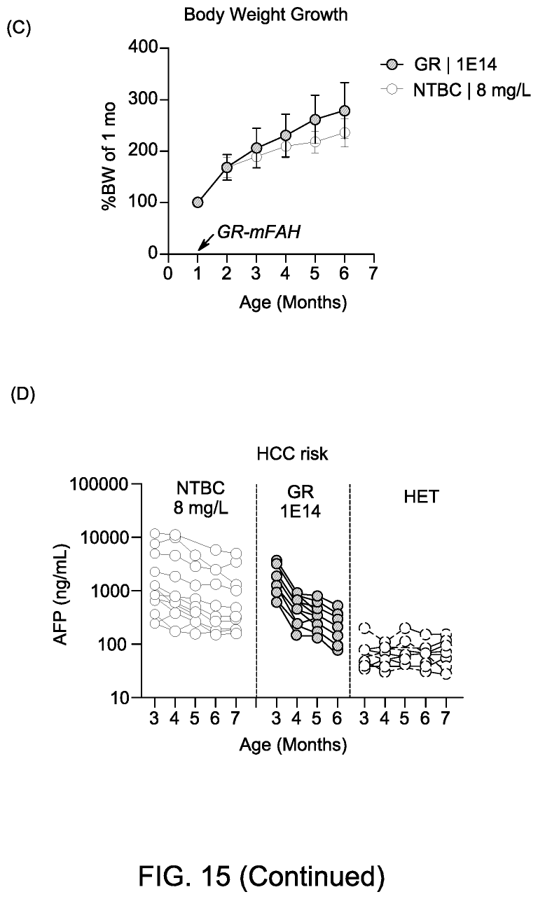

Viral vectors comprising an AAV-DJ viral capsid, mouse FAH (mFAH) transgene, P2A sequence, a flanking 5′ homology arm 1000 nucleotides (nt) in length, and a 3′ homology arm 1600 nt in length were constructed (FIG. 14, panel A). Homology arms were designed for complementarity to a mouse genomic albumin target integration site. Vectors herein described were administered to Fah−/− mice via intravenous injection at 1 month of age (see FIG. 15, panel A). Mice were treated with 8 mg/L of NTBC from birth until administration of GeneRide vectors. From 1 month of age until sacrifice (at least 12 month of age), NTBC administration was discontinued. Mice were assessed for circulating biomarkers (e.g., levels of ALB-2A) for at least 5 months of age (FIG. 15, panel B) and body weight was monitored for at least 6 months post treatment (FIG. 15, panel C). HCC risk (AFP level) was also assessed in these mice (at least 6 months of age) (FIG. 15, panel D).

Among other things, this example demonstrates that treatment of a subject (e.g., a subject suffering from hereditary tyrosinemia type 1) with viral vectors of the present disclosure may provide a rapid selective advantage for cells (e.g. liver cells), leading to complete repopulation of the diseased liver within 4 weeks post-treatment. In some embodiments, treatment of a subject (e.g., a subject suffering from hereditary tyrosinemia) with viral vectors of the present disclosure may enable more than 50% (e.g., 60%, 70%, 80%, 90%, 95%, 99%, 100%, etc.) of cells (e.g., liver cells) within a particular tissue type (e.g. liver) to consist of cells that have successfully integrated a delivered transgene (e.g., FAH) within 4 weeks post-treatment. In some embodiments, treatment a subject (e.g., a subject suffering from hereditary tyrosinemia) with viral vectors of the present disclosure at certain doses (e.g., 3E13 vg/kg, 1E13 vg/kg, 1E14 vg/kg) may provide a selective advantage for cells (e.g. liver cells) within 4 weeks post-treatment.

Among other things, this example demonstrates that treatment with viral vectors comprising a sequence encoding mouse FAH (mFAH) may provide improved liver function in a subject suffering from hereditary tyrosinemia 1 (HT1) (e.g., in a FAH−/− mouse model system) as compared to a reference (e.g., untreated) (FIG. 14, panel B). In some embodiments, as demonstrated in FIG. 14, panel D, treatment of a subject with viral vectors of the present disclosure may provide reduced levels of biomarkers associated with reduced liver function (e.g., ALT) as compared to a reference (e.g., untreated). In some embodiments, as demonstrated in FIG. 14, panel E, treatment of a subject (e.g., a subject suffering from hereditary tyrosinemia) with viral vectors of the present disclosure may provide reduced levels of a harmful metabolite (e.g., SUAC) relative to a reference (e.g., untreated or NTBC-treated).

Among other things, this example demonstrates that treatment of a subject (e.g., a subject suffering from hereditary tyrosinemia) with viral vectors of the present disclosure may show continued transgene expression as adults. In some embodiments, as demonstrated in FIG. 15, panel B, administration of viral vectors of the present disclosure to a subject at least one month of age demonstrated continued expression of circulating biomarker (e.g., ALB-2A) at least 5 months post-dosing. In some embodiments, as demonstrated in FIG. 15, panel C, subjects treated with viral vectors of the present disclosure showed similar body weight as compared to a reference (e.g., NTBC-treated subjects).

Among other things, this example demonstrates that treatment of a subject (e.g., a subject suffering from hereditary tyrosinemia) with viral vectors of the present disclosure may lower HCC risk as compared to a reference (e.g., untreated or NTBC-treated subjects In some embodiments, as demonstrated in FIG. 15, panel D, treatment of a subject (e.g., a subject suffering from hereditary tyrosinemia) with viral vectors of the present disclosure may provide reduced levels of a biomarker (e.g., AFP) associated with a disease (e.g., cancer, including HCC) as compared to a reference (e.g., untreated or NTBC-treated subjects) at least 6 months of age.

### EQUIVALENTS

Those skilled in the art will recognize, or be able to ascertain using no more than routine experimentation, many equivalents to the specific embodiments of the invention described herein. The scope of the present invention is not intended to be limited to the above Description, but rather is as set forth in the following claims:

1. A recombinant viral vector for integrating a transgene into a target integration site in the genome of a cell, comprising:
(i) a polynucleotide comprising a first nucleic acid sequence and a second nucleic acid sequence, wherein the first nucleic acid sequence comprises at least one exogenous gene sequence and the second nucleic acid sequence is positioned 5′ or 3′ to the first nucleic acid sequence and promotes the production of two independent gene products upon integration into a target integration site;
(ii) a third nucleic acid sequence positioned 5′ to the polynucleotide and comprising a sequence that is homologous to a genomic sequence 5′ of the target integration site; and
(iii) a fourth nucleic acid sequence positioned 3′ to the polynucleotide and comprising a sequence that is homologous to a genomic sequence 3′ of the target integration site;
wherein each of the third nucleic acid sequence and fourth nucleic acid sequence are at least 1000 nucleotides (nt) in length and wherein the third nucleic acid sequence and fourth nucleic acid sequence are different lengths.

2. The recombinant viral vector of claim 1, wherein:
(i) the exogenous gene sequence is between 500 nt and 2500 nt in length, or
(ii) the exogenous gene sequence is between 1000 nt and 2000 nt in length.

3. (canceled)

4. The recombinant viral vector of claim 1, wherein:
(i) the third nucleic acid sequence is at least 750 nt in length;
(ii) the third nucleic acid sequence is at least 1000 nt in length; or
(iii) the third nucleic acid sequence is at least 1600 nt in length.

5-6. (canceled)

7. The recombinant viral vector of claim 1, wherein:
(i) the fourth nucleic acid sequence is at least 750 nt in length;
(ii) the fourth nucleic acid sequence is at least 1000 nt in length; or
(iii) the fourth nucleic acid sequence is at least 1600 nt in length.

8-9. (canceled)

10. The recombinant viral vector of claim 1, wherein;
(i) both the third and fourth nucleic acid sequences are at least 750 nt in length; or
(ii) both the third and fourth nucleic acid sequences are at least 1000 nt in length.

11. (canceled)

12. The recombinant viral vector of claim 1, wherein;
(i) the third nucleic acid sequence is 1000 nt in length and the fourth nucleic acid sequence is 1600 nt in length; or
(ii) the third nucleic acid sequence is 1600 nt in length and the fourth nucleic acid sequence is 1000 nt in length.

13. (canceled)

14. The recombinant viral vector of claim 1, wherein the viral vector is an AAV vector.

15. The recombinant viral vector of claim 14, wherein the viral vector is selected from AAV2, AAV8, AAV9, AAV-DJ, AAV-LK03, or AAV-sL65.

16. A composition comprising:
a recombinant viral vector of claim 1; and
one or more pharmaceutically acceptable excipients.

17. A method of integrating a transgene into the genome of one or more cells in a tissue in a subject, said method comprising administering to a subject in which cells in the tissue fail to express a functional protein encoded by a gene product, a composition of claim 16, wherein the target integration site is in the genome of the one or more cells.

18. The method according to claim 17, wherein:
(i) the one or more cells are non-dividing cells; and/or
(ii) the one or more cells are liver, muscle, lung, or CNS cells.

19. (canceled)

20. The method of claim 17, wherein the composition is administered during a postnatal period.

21. The method of claim 17, wherein:
(i) the composition is administered at least 7 days postnatal, at least 14 days postnatal, at least 21 days postnatal, or at least 28 days postnatal; or
(ii) the composition is administered at or before 28 days postnatal.

22. (canceled)

23. The method of claim 17, wherein the transgene is or comprises UGT1A1, FAH, Factor IX, A1AT, ASL, or LIPA.

24. The method of claim 17, wherein integration efficiency is improved relative to a reference composition.

25. The method of claim 17, wherein:
(i) the composition is administered at a dosage of at least 5E13 viral genomes per kilogram (vg/kg);
(ii) the composition is administered at a dosage of 1E14 vg/kg; or
(iii) the composition is administered at a dosage of 2E14 vg/kg.

26-27. (canceled)

28. The method of claim 17, wherein the subject is an animal and/or a human.

29. (canceled)

30. The method of claim 28, wherein the subject has or is suspected to have Crigler-Najjar syndrome, tyrosinemia, hemophilia, alpha-1 antitrypsin deficiency, argininosuccinic aciduria, or lysosomal acid lipase deficiency.

31. A method of determining homology arm lengths for a polynucleotide cassette comprising at least one payload, a 5′ homology arm, and a 3′ homology arm comprising
(a) determining the length of a polynucleotide cassette comprising at least one payload but not including any homology arms,
(b) if the lengths of step (a) is less than 2.7 kilobases (kb), then the length of the 3′ homology arm sequence is 1 kb or less and the length of the 5′ homology arm sequence is 2.7 kb—the length of the 3′ homology arm, and
(c) if the length of step (a) is greater than 2.7 kb, then the 5′ and 3′ homology arms are each [4.7 kb—the length of step (a)]/2 nucleotides in length

32. A recombinant viral vector for integrating a transgene into a target integration site in the genome of a cell, comprising:
(i) a polynucleotide comprising a first nucleic acid sequence and a second nucleic acid sequence, wherein the first nucleic acid sequence comprises at least one exogenous gene sequence and the second nucleic acid sequence is positioned 5′ or 3′ to the first nucleic acid sequence and promotes the production of two independent gene products upon integration into a target integration site;
(ii) a third nucleic acid sequence positioned 5′ to the polynucleotide and comprising a sequence that is homologous to a genomic sequence 5′ of the target integration site; and
(iii) a fourth nucleic acid sequence positioned 3′ to the polynucleotide and comprising a sequence that is homologous to a genomic sequence 3′ of the target integration site;
wherein the length of each of the third nucleic acid sequence and fourth nucleic acid sequence are determined according to claim 31.


SEQUENCE LISTING (12 sequences)

SEQ ID NO: 1 - DNA, 7393 bp, Artificial Sequence
  Synthetic AAV-DJ-mha-mFAH (PM-0550)
cagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtaatgcatggatcccctagggcggccgcctgaaactagacaaaacccgtgtgactggcatcgattattctatttgatctagctagtcctagcaaagtgacaactgctactcccctcctacacagccaagattcctaagttggcagtggcatgcttaatcctcaaagccaaagttacttggctccaagatttatagccttaaactgtggcctcacattccttcctatcttactttcctgcactggggtaaatgtctccttgctcttcttgctttctgtcctactgcagggctcttgctgagctggtgaagcacaagcccaaggctacagcggagcaactgaagactgtcatggatgactttgcacagttcctggatacatgttgcaaggctgctgacaaggacacctgcttctcgactgaggtcagaaacgtttttgcattttgacgatgttcagtttccattttctgtgcacgtggtcaggtgtagctctctggaactcacacactgaataactccaccaatctagatgttgttctctacgtaactgtaatagaaactgacttacgtagcttttaatttttattttctgccacactgctgcctattaaatacctattatcactatttggtttcaaatttgtgacacagaagagcatagttagaaatacttgcaaagcctagaatcatgaactcatttaaaccttgccctgaaatgtttctttttgaattgagttattttacacatgaatggacagttaccattatatatctgaatcatttcacattccctcccatggcctaacaacagtttatcttcttattttgggcacaacagatgtcagagagcctgctttaggaattctaagtagaactgtaattaagcaatgcaaggcacgtacgtttactatgtcattgcctatggctatgaagtgcaaatcctaacagtcctgctaatacttttctaacatccatcatttctttgttttcagggtccaaaccttgtcactagatgcaaagacgccttagccggaagcggcgccaccaatttcagcctgctgaaacaggccggcgacgtggaagagaaccctggcccttcctttattccagtggccgaggactccgactttcccatccaaaacctgccctatggtgttttctccactcaaagcaacccaaagccacggattggtgtagccatcggtgaccagatcttggacctgagtgtcattaaacacctctttaccggacctgccctttccaaacatcaacatgtcttcgatgagacaactctcaataacttcatgggtctgggtcaagctgcatggaaggaggcaagagcatccttacagaacttactgtctgccagccaagcccggctcagagatgacaaggagcttcggcagcgtgcattcacctcccaggcttctgcgacaatgcaccttcctgctaccataggagactacacggacttctactcttctcggcagcatgccaccaatgttggcattatgttcagaggcaaggagaatgcgctgttgccaaattggctccacttacctgtgggataccatggccgagcttcctccattgtggtatctggaaccccgattcgaagacccatggggcagatgagacctgataactcaaagcctcctgtgtatggtgcctgcagactcttagacatggagttggaaatggctttcttcgtaggccctgggaacagattcggagagccaatccccatttccaaagcccatgaacacattttcgggatggtcctcatgaacgactggagcgcacgagacatccagcaatgggagtacgtcccacttgggccattcctggggaaaagctttggaaccacaatctccccgtgggtggtgcctatggatgccctcatgccctttgtggtgccaaacccaaagcaggaccccaagcccttgccatatctctgccacagccagccctacacatttgatatcaacctgtctgtctctttgaaaggagaaggaatgagccaggcggctaccatctgcaggtctaactttaagcacatgtactggaccatgctgcagcaactcacacaccactctgttaatggatgcaacctgagacctggggacctcttggcttctggaaccatcagtggatcagaccctgaaagctttggctccatgctggaactgtcctggaagggaacaaaggccatcgatgtggggcaggggcagaccaggaccttcctgctggacggcgatgaagtcatcataacaggtcactgccagggggacggctaccgtgttggctttggccagtgtgctgggaaagtgctgcctgccctttcaccagcctaaacacatcacaaccacaaccttctcaggtaactatacttgggacttaaaaaacataatcataatcatttttcctaaaacgatcaagactgataaccatttgacaagagccatacagacaagcaccagctggcactcttaggtcttcacgtatggtcatcagtttgggttccatttgtagataagaaactgaacatataaaggtctaggttaatgcaatttacacaaaaggagaccaaaccagggagagaaggaaccaaaattaaaaattcaaaccagagcaaaggagttagccctggttttgctctgacttacatgaaccactatgtggagtcctccatgttagcctagtcaagcttatcctctggatgaagttgaaaccatatgaaggaatatttggggggtgggtcaaaacagttgtgtatcaatgattccatgtggtttgacccaatcattctgtgaatccatttcaacagaagatacaacgggttctgtttcataataagtgatccacttccaaatttctgatgtgccccatgctaagctttaacagaatttatcttcttatgacaaagcagcctcctttgaaaatatagccaactgcacacagctatgttgatcaattttgtttataatcttgcagaagagaattttttaaaatagggcaataatggaaggctttggcaaaaaaattgtttctccatatgaaaacaaaaaacttatttttttattcaagcaaagaacctatagacataaggctatttcaaaattatttcagttttagaaagaattgaaagttttgtagcattctgagaagacagctttcatttgtaatcataggtaatatgtaggtcctcagaaatggtgagacccctgactttgacacttggggactctgagggaccagtgatgaagagggcacaacttatatcacacatgcacgagttggggtgagagggtgtcacaacatctatcagtgtgtcatctgcccaccaagtaacagatgtcagctaagactaggtcatgtgtaggctgtctacaccagtgaaaatcgcaaaaagaatctaagaaattccacatttctagaaaataggtttggaaaccgtattccattttacaaaggacacttacatttctctttttgttttccaggctaccctgagaaaaaaagacatgaagactcaggactcatcttttctgttggtgtaaaatcaacaccctaaggaacacaaatttctttaaacatttgacttcttgtctctgtgctgcaattaataaaaaatggaaagaatctactctgtggttcagaactctatcttccaaaggcgcgcttcaccctagcagcctctttggctcagaggaatccctgcctttcctcccttcatctcagcagagaatgtagttccacatgggcaacacaatgaaaataaacgttaatactctcccatcttatgggtggtgaccctagaaaccaatacttcaacattacgagaattctgaatgagagactaaaagcttatgaactgtggctttcctttgtcagtgggactctaagaatgagttggggacaaaagagataggaatggctttaaaggtgactagttgaactgataaagtaaatgaactgaggaaaaaaaatatcactcaacctgcaggggacgtcctacgtaatgcataggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagc

SEQ ID NO: 2 - DNA, 7393 bp, Artificial Sequence
  Synthetic AAV-DJ-mha-hFAH (PM-0549)
cagatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgaatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagccatattcaacgggaaacgtcgaggccgcgattaaattccaacatggatgctgatttatatgggtataaatgggctcgcgataatgtcgggcaatcaggtgcgacaatctatcgcttgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccggaaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataaacttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttacgtaatgcatggatcccctagggcggccgcctgaaactagacaaaacccgtgtgactggcatcgattattctatttgatctagctagtcctagcaaagtgacaactgctactcccctcctacacagccaagattcctaagttggcagtggcatgcttaatcctcaaagccaaagttacttggctccaagatttatagccttaaactgtggcctcacattccttcctatcttactttcctgcactggggtaaatgtctccttgctcttcttgctttctgtcctactgcagggctcttgctgagctggtgaagcacaagcccaaggctacagcggagcaactgaagactgtcatggatgactttgcacagttcctggatacatgttgcaaggctgctgacaaggacacctgcttctcgactgaggtcagaaacgtttttgcattttgacgatgttcagtttccattttctgtgcacgtggtcaggtgtagctctctggaactcacacactgaataactccaccaatctagatgttgttctctacgtaactgtaatagaaactgacttacgtagcttttaatttttattttctgccacactgctgcctattaaatacctattatcactatttggtttcaaatttgtgacacagaagagcatagttagaaatacttgcaaagcctagaatcatgaactcatttaaaccttgccctgaaatgtttctttttgaattgagttattttacacatgaatggacagttaccattatatatctgaatcatttcacattccctcccatggcctaacaacagtttatcttcttattttgggcacaacagatgtcagagagcctgctttaggaattctaagtagaactgtaattaagcaatgcaaggcacgtacgtttactatgtcattgcctatggctatgaagtgcaaatcctaacagtcctgctaatacttttctaacatccatcatttctttgttttcagggtccaaaccttgtcactagatgcaaagacgccttagccggaagcggcgccaccaatttcagcctgctgaaacaggccggcgacgtggaagagaaccctggcccttccttcatcccggtggccgaggattccgacttccccatccacaacctgccctacggcgtcttctcgaccagaggcgacccaagaccgaggataggtgtggccattggcgaccagatcctggacctcagcatcatcaagcacctctttactggtcctgtcctctccaaacaccaggatgtcttcaatcagcctacactcaacagcttcatgggcctgggtcaggctgcctggaaggaggcgagagtgttcttgcagaacttgctgtctgtgagccaagccaggctcagagatgacaccgaacttcggaagtgtgcattcatctcccaggcttctgccacgatgcaccttccagccaccataggagactacacagacttctattcctctcggcagcatgctaccaacgtcggaatcatgttcagggacaaggagaatgcgttgatgccaaattggctgcacttaccagtgggctaccatggccgtgcctcctctgtcgtggtgtctggcaccccaatccgaaggcccatgggacagatgaaacctgatgactctaagcctcccgtatatggtgcctgcaagctcttggacatggagctggaaatggctttttttgtaggccctggaaacagattgggagagccgatccccatttccaaggcccatgagcacatttttggaatggtccttatgaacgactggagtgcacgagacattcagaagtgggagtatgtccctctcgggccattccttgggaagagttttgggaccactgtctctccgtgggtggtgcccatggatgctctcatgccctttgctgtgcccaacccgaagcaggaccccaggcccctgccgtatctgtgccatgacgagccctacacatttgacatcaacctctctgttaacctgaaaggagaaggaatgagccaggcggctaccatatgcaagtccaattttaagtacatgtactggacgatgctgcagcagctcactcaccactctgtcaacggctgcaacctgcggccgggggacctcctggcttctgggaccatcagcgggccggagccagaaaacttcggctccatgttggaactgtcgtggaagggaacgaagcccatagacctggggaatggtcagaccaggaagtttctgctggacggggatgaagtcatcataacagggtactgccagggggatggttaccgcatcggctttggccagtgtgctggaaaagtgctgcctgctctcctgccatcataaacacatcacaaccacaaccttctcaggtaactatacttgggacttaaaaaacataatcataatcatttttcctaaaacgatcaagactgataaccatttgacaagagccatacagacaagcaccagctggcactcttaggtcttcacgtatggtcatcagtttgggttccatttgtagataagaaactgaacatataaaggtctaggttaatgcaatttacacaaaaggagaccaaaccagggagagaaggaaccaaaattaaaaattcaaaccagagcaaaggagttagccctggttttgctctgacttacatgaaccactatgtggagtcctccatgttagcctagtcaagcttatcctctggatgaagttgaaaccatatgaaggaatatttggggggtgggtcaaaacagttgtgtatcaatgattccatgtggtttgacccaatcattctgtgaatccatttcaacagaagatacaacgggttctgtttcataataagtgatccacttccaaatttctgatgtgccccatgctaagctttaacagaatttatcttcttatgacaaagcagcctcctttgaaaatatagccaactgcacacagctatgttgatcaattttgtttataatcttgcagaagagaattttttaaaatagggcaataatggaaggctttggcaaaaaaattgtttctccatatgaaaacaaaaaacttatttttttattcaagcaaagaacctatagacataaggctatttcaaaattatttcagttttagaaagaattgaaagttttgtagcattctgagaagacagctttcatttgtaatcataggtaatatgtaggtcctcagaaatggtgagacccctgactttgacacttggggactctgagggaccagtgatgaagagggcacaacttatatcacacatgcacgagttggggtgagagggtgtcacaacatctatcagtgtgtcatctgcccaccaagtaacagatgtcagctaagactaggtcatgtgtaggctgtctacaccagtgaaaatcgcaaaaagaatctaagaaattccacatttctagaaaataggtttggaaaccgtattccattttacaaaggacacttacatttctctttttgttttccaggctaccctgagaaaaaaagacatgaagactcaggactcatcttttctgttggtgtaaaatcaacaccctaaggaacacaaatttctttaaacatttgacttcttgtctctgtgctgcaattaataaaaaatggaaagaatctactctgtggttcagaactctatcttccaaaggcgcgcttcaccctagcagcctctttggctcagaggaatccctgcctttcctcccttcatctcagcagagaatgtagttccacatgggcaacacaatgaaaataaacgttaatactctcccatcttatgggtggtgaccctagaaaccaatacttcaacattacgagaattctgaatgagagactaaaagcttatgaactgtggctttcctttgtcagtgggactctaagaatgagttggggacaaaagagataggaatggctttaaaggtgactagttgaactgataaagtaaatgaactgaggaaaaaaaatatcactcaacctgcaggggacgtcctacgtaatgcataggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaagaattaattccgtgtattctatagtgtcacctaaatcgtatgtgtatgatacataaggttatgtattaattgtagccgcgttctaacgacaatatgtacaagcctaattgtgtagcatctggcttactgaagcagaccctatcatctctctcgtaaactgccgtcagagtcggtttggttggacgaaccttctgagtttctggtaacgccgtcccgcacccggaaatggtcagcgaaccaatcagcagggtcatcgctagc

SEQ ID NO: 3 - DNA, 10221 bp, Artificial Sequence
  Synthetic AAV-DJ-mha-mFAH (PM-0549)
gtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtctaagaaaccattattatcatgacattaacctataaaaataggcgtatcacgaggccctttcgtctcgcgcgtttcggtgatgacggtgaaaacctctgacacatgcagctcccggagacggtcacagcttgtctgtaagcggatgccgggagcagacaagcccgtcagggcgcgtcagcgggtgttggcgggtgtcggggctggcttaactatgcggcatcagagcagattgtactgagagtgcaccattcgacgctctcccttatgcgactcctgcattaggaagcagcccagtagtaggttgaggccgttgagcaccgccgccgcaaggaatggtgcatgcaaggagatggcgcccaacagtcccccggccacggggcctgccaccatacccacgccgaaacaagcgctcatgagcccgaagtggcgagcccgatcttccccatcggtgatgtcggcgatataggcgccagcaaccgcacctgtggcgccggtgggtcaccaagcaggaagtcaaagactttttccggtgggcaaaggatcacgtggttgaggtggagcatgaattctacgtcaaaaagggtggagccaagaaaagacccgcccccagtgacgcagatataagtgagcccaaacgggtgcgcgagtcagttgcgcagccatcgacgtcagacgcggaagcttcgatcaactacgcagacaggtaccaaaacaaatgttctcgtcacgtgggcatgaatctgatgctgtttccctgcagacaatgcgagagaatgaatcagaattcaaatatctgcttcactcacggacagaaagactgtttagagtgctttcccgtgtcagaatctcaacccgtttctgtcgtcaaaaaggcgtatcagaaactgtgctacattcatcatatcatgggaaaggtgccagacgcttgcactgcctgcgatctggtcaatgtggatttggatgactgcatctttgaacaataaatgatttaaatcaggtatggctgccgatggttatcttccagattggctcgaggacactctctctgaaggaataagacagtggtggaagctcaaacctggcccaccaccaccaaagcccgcagagcggcataaggacgacagcaggggtcttgtgcttcctgggtacaagtacctcggacccttcaacggactcgacaagggagagccggtcaacgaggcagacgccgcggccctcgagcacgacaaagcctacgaccggcagctcgacagcggagacaacccgtacctcaagtacaaccacgccgacgccgagttccaggagcggctcaaagaagatacgtcttttgggggcaacctcgggcgagcagtcttccaggccaaaaagaggcttcttgaacctcttggtctggttgaggaagcggctaagacggctcctggaaagaagaggcctgtagagcactctcctgtggagccagactcctcctcgggaaccggaaaggcgggccagcagcctgcaagaaaaagattgaattttggtcagactggagacgcagactcagtcccagaccctcaaccaatcggagaacctcccgcagccccctcaggtgtgggatctcttacaatggctgcaggcggtggcgcaccaatggcagacaataacgagggcgccgacggagtgggtaattcctcgggaaattggcattgcgattccacatggatgggcgacagagtcatcaccaccagcacccgaacctgggccctgcccacctacaacaaccacctctacaagcaaatctccaacagcacatctggaggatcttcaaatgacaacgcctacttcggctacagcaccccctgggggtattttgactttaacagattccactgccacttttcaccacgtgactggcagcgactcatcaacaacaactggggattccggcccaagagactcagcttcaagctcttcaacatccaggtcaaggaggtcacgcagaatgaaggcaccaagaccatcgccaataacctcaccagcaccatccaggtgtttacggactcggagtaccagctgccgtacgttctcggctctgcccaccagggctgcctgcctccgttcccggcggacgtgttcatgattccccagtacggctacctaacactcaacaacggtagtcaggccgtgggacgctcctccttctactgcctggaatactttccttcgcagatgctgagaaccggcaacaacttccagtttacttacaccttcgaggacgtgcctttccacagcagctacgcccacagccagagcttggaccggctgatgaatcctctgattgaccagtacctgtactacttgtctcggactcaaacaacaggaggcacgacaaatacgcagactctgggcttcagccaaggtgggcctaatacaatggccaatcaggcaaagaactggctgccaggaccctgttaccgccagcagcgagtatcaaagacatctgcggataacaacaacagtgaatactcgtggactggagctaccaagtaccacctcaatggcagagactctctggtgaatccgggcccggccatggcaagccacaaggacgatgaagaaaagttttttcctcagagcggggttctcatctttgggaagcaaggctcagagaaaacaaatgtggacattgaaaaggtcatgattacagacgaagaggaaatcaggacaaccaatcccgtggctacggagcagtatggttctgtatctaccaacctccagagaggcaacagacaagcagctaccgcagatgtcaacacacaaggcgttcttccaggcatggtctggcaggacagagatgtgtaccttcaggggcccatctgggcaaagattccacacacggacggacattttcacccctctcccctcatgggtggattcggacttaaacaccctccgcctcagatcctgatcaagaacacgcctgtacctgcggatcctccgaccaccttcaaccagtcaaagctgaactctttcatcacccagtattctactggccaagtcagcgtggagatcgagtgggagctgcagaaggaaaacagcaagcgctggaaccccgagatccagtacacctccaactactacaaatctacaagtgtggactttgctgttaatacagaaggcgtgtactctgaaccccgccccattggcacccgttacctcacccgtaatctgtaattgcttgttaatcaataaaccgtttaattcgtttcagttgaactttggtctctgcgtatttctttcttatctagtttccatatgcatgtagataagtagcatggcgggttaatcattaactaaccggtacctctagaactatagctagcgatgaccctgctgattggttcgctgaccatttccgggtgcgggacggcgttaccagaaactcagaaggttcgtccaaccaaaccgactctgacggcagtttacgagagagatgatagggtctgcttcagtaagccagatgctacacaattaggcttgtacatattgtcgttagaacgcggctacaattaatacataaccttatgtatcatacacatacgatttaggtgacactatagaatacacggaattaattcttggccactccctctctgcgcgctcgctcgctcactgaggccgcccgggcaaagcccgggcgtcgggcgacctttggtcgcccggcctcagtgagcgagcgagcgcgcagagagggagtggccaactccatcactaggggttccttaccgactgaaactagacaaaacccgtgtgactggcatcgattattctatttgatctagctagtcctagcaaagtgacaactgctactcccctcctacacagccaagattcctaagttggcagtggcatgcttaatcctcaaagccaaagttacttggctccaagatttatagccttaaactgtggcctcacattccttcctatcttactttcctgcactggggtaaatgtctccttgctcttcttgctttctgtcctactgcagggctcttgctgagctggtgaagcacaagcccaaggctacagcggagcaactgaagactgtcatggatgactttgcacagttcctggatacatgttgcaaggctgctgacaaggacacctgcttctcgactgaggtcagaaacgtttttgcattttgacgatgttcagtttccattttctgtgcacgtggtcaggtgtagctctctggaactcacacactgaataactccaccaatctagatgttgttctctacgtaactgtaatagaaactgacttacgtagcttttaatttttattttctgccacactgctgcctattaaatacctattatcactatttggtttcaaatttgtgacacagaagagcatagttagaaatacttgcaaagcctagaatcatgaactcatttaaaccttgccctgaaatgtttctttttgaattgagttattttacacatgaatggacagttaccattatatatctgaatcatttcacattccctcccatggcctaacaacagtttatcttcttattttgggcacaacagatgtcagagagcctgctttaggaattctaagtagaactgtaattaagcaatgcaaggcacgtacgtttactatgtcattgcctatggctatgaagtgcaaatcctaacagtcctgctaatacttttctaacatccatcatttctttgttttcagggtccaaaccttgtcactagatgcaaagacgccttagccggaagcggcgccaccaatttcagcctgctgaaacaggccggcgacgtggaagagaaccctggcccttcctttattccagtggccgaggactccgactttcccatccaaaacctgccctatggtgttttctccactcaaagcaacccaaagccacggattggtgtagccatcggtgaccagatcttggacctgagtgtcattaaacacctctttaccggacctgccctttccaaacatcaacatgtcttcgatgagacaactctcaataacttcatgggtctgggtcaagctgcatggaaggaggcaagagcatccttacagaacttactgtctgccagccaagcccggctcagagatgacaaggagcttcggcagcgtgcattcacctcccaggcttctgcgacaatgcaccttcctgctaccataggagactacacggacttctactcttctcggcagcatgccaccaatgttggcattatgttcagaggcaaggagaatgcgctgttgccaaattggctccacttacctgtgggataccatggccgagcttcctccattgtggtatctggaaccccgattcgaagacccatggggcagatgagacctgataactcaaagcctcctgtgtatggtgcctgcagactcttagacatggagttggaaatggctttcttcgtaggccctgggaacagattcggagagccaatccccatttccaaagcccatgaacacattttcgggatggtcctcatgaacgactggagcgcacgagacatccagcaatgggagtacgtcccacttgggccattcctggggaaaagctttggaaccacaatctccccgtgggtggtgcctatggatgccctcatgccctttgtggtgccaaacccaaagcaggaccccaagcccttgccatatctctgccacagccagccctacacatttgatatcaacctgtctgtctctttgaaaggagaaggaatgagccaggcggctaccatctgcaggtctaactttaagcacatgtactggaccatgctgcagcaactcacacaccactctgttaatggatgcaacctgagacctggggacctcttggcttctggaaccatcagtggatcagaccctgaaagctttggctccatgctggaactgtcctggaagggaacaaaggccatcgatgtggggcaggggcagaccaggaccttcctgctggacggcgatgaagtcatcataacaggtcactgccagggggacggctaccgtgttggctttggccagtgtgctgggaaagtgctgcctgccctttcaccagcctaaacacatcacaaccacaaccttctcaggtaactatacttgggacttaaaaaacataatcataatcatttttcctaaaacgatcaagactgataaccatttgacaagagccatacagacaagcaccagctggcactcttaggtcttcacgtatggtcatcagtttgggttccatttgtagataagaaactgaacatataaaggtctaggttaatgcaatttacacaaaaggagaccaaaccagggagagaaggaaccaaaattaaaaattcaaaccagagcaaaggagttagccctggttttgctctgacttacatgaaccactatgtggagtcctccatgttagcctagtcaagcttatcctctggatgaagttgaaaccatatgaaggaatatttggggggtgggtcaaaacagttgtgtatcaatgattccatgtggtttgacccaatcattctgtgaatccatttcaacagaagatacaacgggttctgtttcataataagtgatccacttccaaatttctgatgtgccccatgctaagctttaacagaatttatcttcttatgacaaagcagcctcctttgaaaatatagccaactgcacacagctatgttgatcaattttgtttataatcttgcagaagagaattttttaaaatagggcaataatggaaggctttggcaaaaaaattgtttctccatatgaaaacaaaaaacttatttttttattcaagcaaagaacctatagacataaggctatttcaaaattatttcagttttagaaagaattgaaagttttgtagcattctgagaagacagctttcatttgtaatcataggtaatatgtaggtcctcagaaatggtgagacccctgactttgacacttggggactctgagggaccagtgatgaagagggcacaacttatatcacacatgcacgagttggggtgagagggtgtcacaacatctatcagtgtgtcatctgcccaccaagtaacagatgtcagctaagactaggtcatgtgtaggctgtctacaccagtgaaaatcgcaaaaagaatctaagaaattccacatttctagaaaataggtttggaaaccgtattccattttacaaaggacacttacatttctctttttgttttccaggctaccctgagaaaaaaagacatgaagactcaggactcatcttttctgttggtgtaaaatcaacaccctaaggaacacaaatttctttaaacatttgacttcttgtctctgtgctgcaattaataaaaaatggaaagaatctactctgtggttcagaactctatcttccaaaggcgcgcttcaccctagcagcctctttggctcagaggaatccctgcctttcctcccttcatctcagcagagaatgtagttccacatgggcaacacaatgaaaataaacgttaatactctcccatcttatgggtggtgaccctagaaaccaatacttcaacattacgagaattctgaatgagagactaaaagcttatgaactgtggctttcctttgtcagtgggactctaagaatgagttggggacaaaagagataggaatggctttaaaggtgactagttgaactgataaagtaaatgaactgaggaaaaaaaatatcactcaatcggtaaggaacccctagtgatggagttggccactccctctctgcgcgctcgctcgctcactgaggccgggcgaccaaaggtcgcccgacgcccgggctttgcccgggcggcctcagtgagcgagcgagcgcgcagagagggagtggccaactttttgcaaaagcctaggcctccaaaaaagcctcctcactacttctggaatagctcagaggccgaggcggcctcggcctctgcataaataaaaaaaattagtcagccatggggcggagaatgggcggaactgggcggagttaggggcgggatgggcggagttaggggcgggactatggttgctgactaattgagatgcatgctttgcatacttctgcctgctggggagcctggggactttccacacctggttgctgactaattgagatgcatgctttgcatacttctgcctgctggggagcctggggactttccacaccctaactgacacacattccacagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaa

SEQ ID NO: 4 - DNA, 1254 bp, Homo sapiens
  misc_feature: Human FAH
tccttcatcccggtggccgaggattccgacttccccatccacaacctgccctacggcgtcttctcgaccagaggcgacccaagaccgaggataggtgtggccattggcgaccagatcctggacctcagcatcatcaagcacctctttactggtcctgtcctctccaaacaccaggatgtcttcaatcagcctacactcaacagcttcatgggcctgggtcaggctgcctggaaggaggcgagagtgttcttgcagaacttgctgtctgtgagccaagccaggctcagagatgacaccgaacttcggaagtgtgcattcatctcccaggcttctgccacgatgcaccttccagccaccataggagactacacagacttctattcctctcggcagcatgctaccaacgtcggaatcatgttcagggacaaggagaatgcgttgatgccaaattggctgcacttaccagtgggctaccatggccgtgcctcctctgtcgtggtgtctggcaccccaatccgaaggcccatgggacagatgaaacctgatgactctaagcctcccgtatatggtgcctgcaagctcttggacatggagctggaaatggctttttttgtaggccctggaaacagattgggagagccgatccccatttccaaggcccatgagcacatttttggaatggtccttatgaacgactggagtgcacgagacattcagaagtgggagtatgtccctctcgggccattccttgggaagagttttgggaccactgtctctccgtgggtggtgcccatggatgctctcatgccctttgctgtgcccaacccgaagcaggaccccaggcccctgccgtatctgtgccatgacgagccctacacatttgacatcaacctctctgttaacctgaaaggagaaggaatgagccaggcggctaccatatgcaagtccaattttaagtacatgtactggacgatgctgcagcagctcactcaccactctgtcaacggctgcaacctgcggccgggggacctcctggcttctgggaccatcagcgggccggagccagaaaacttcggctccatgttggaactgtcgtggaagggaacgaagcccatagacctggggaatggtcagaccaggaagtttctgctggacggggatgaagtcatcataacagggtactgccagggggatggttaccgcatcggctttggccagtgtgctggaaaagtgctgcctgctctcctgccatca

SEQ ID NO: 5 - DNA, 1254 bp, Artificial Sequence
  Synthetic Human FAH Codon optimized variant 3
tccttcattcctgtggccgaggattctgactttcccattcacaacctgccctatggcgtcttttcaaccaggggagatcccaggcccagaatcggggtggccattggagatcagatcctggacctgtcaatcatcaagcacctgtttacaggccctgtgctgagcaagcaccaggatgtgttcaaccagccaactctgaacagcttcatggggctggggcaggctgcctggaaggaggcaagggtgtttctgcagaatctgctgagcgtgtcacaggctaggctgagggatgataccgagctgaggaagtgtgcatttatctcacaggcatcagccacaatgcatctgccagctacaatcggcgactataccgacttttactcaagcaggcagcatgccaccaatgtgggcatcatgttcagggacaaagagaatgccctgatgccaaattggctgcacctgcctgtgggctatcatggcagagccagctcagtggtggtgtcagggacaccaattaggaggcctatgggccagatgaagcctgatgacagcaaaccacctgtctacggcgcctgcaagctcctggatatggaactggagatggctttttttgtgggcccaggcaatagactgggagagcccattccaatctcaaaggcccatgaacatatctttggcatggtcctgatgaatgactggtcagccagagacattcagaagtgggagtacgtgccactgggaccatttctgggaaagagctttggcaccacagtgtcaccatgggtggtgcccatggatgccctgatgccctttgctgtgccaaatccaaaacaagaccccaggcccctcccctatctctgtcatgatgaaccatatacttttgacattaacctgagcgtgaacctgaaaggagaaggcatgagccaagccgccactatctgtaagagcaacttcaaatatatgtactggacaatgctgcagcagctcacccaccatagcgtgaatgggtgtaacctgaggccaggcgacctgctggcatcaggcactatttcagggcctgagcctgaaaattttggatcaatgctggagctgtcatggaagggaactaaacccatcgacctggggaatggccagaccagaaagtttctcctggatggcgatgaggtgatcattacaggctactgtcagggggatggctatagaattggatttggccaatgcgccggcaaagtcctccctgccctgctgcccagc

SEQ ID NO: 6 - DNA, 1254 bp, Artificial Sequence
  Synthetic Human FAH Codon optimized variant 2
tccttcatcccagtggctgaagattctgacttccccattcacaacctcccttatggagtctttagcacaagaggagacccaagacctagaattggcgtggctattggggatcagatcctggacctgagcattatcaaacatctctttacaggcccagtcctcagcaagcaccaagatgtgttcaatcaacccacactgaactcatttatggggctgggccaagctgcctggaaggaggcaagagtgtttctccagaacctcctgtcagtgagccaggccaggctgagagatgacacagagctgaggaagtgtgcctttatctcacaagccagcgccacaatgcacctgccagcaaccatcggggactacacagacttttacagcagcaggcagcacgccactaatgtgggcatcatgtttagagacaaagagaatgccctcatgcctaactggctgcatctgccagtgggctaccatggcagagccagcagcgtggtggtgtctggcacccctatcaggaggcccatgggccagatgaagcctgatgacagcaagccacctgtgtatggggcatgtaagctgctggacatggagctggaaatggctttctttgtggggcctgggaacaggctgggcgaaccaattcccatcagcaaagctcatgaacacatttttgggatggtgctcatgaatgactggtctgccagagacatccagaagtgggagtatgtccccctgggaccattcctgggcaagagctttgggaccactgtgtcaccatgggtggtgcccatggatgccctgatgccatttgctgtgcctaatcccaaacaagatccaaggccactgccctacctctgccacgatgagccctacacatttgatatcaacctctctgtgaatctgaagggagaaggaatgagccaagctgctaccatttgcaaaagcaactttaagtatatgtattggaccatgctgcagcaactcacccaccacagcgtgaatggctgtaacctgaggcctggcgacctgctggcctctggcaccatcagcggccctgaacctgagaactttggcagcatgctggagctgagctggaagggaaccaagcccattgacctgggcaatggacagaccaggaaattcctcctggacggggacgaggtgatcatcacaggctattgccagggggatgggtataggattggctttggccaatgtgccggcaaagtgctgcctgccctgctccctagc

SEQ ID NO: 7 - DNA, 1254 bp, Artificial Sequence
  Synthetic Human FAH Codon optimized variant 1
tcctttattcctgtggccgaagatagcgacttccctatccataacctcccatatggcgtcttctcaaccaggggcgaccccaggcccaggattggggtggcaattggagaccagatcctggacctcagcatcattaagcacctctttacaggacctgtgctgagcaagcaccaggatgtgttcaaccagcctaccctgaatagctttatgggactgggccaggcagcatggaaagaagccagagtgttcctccagaatctgctgagcgtgagccaggccaggctcagggatgacacagaactgaggaaatgtgccttcatctcacaggcatcagccacaatgcacctccctgccacaattggggactacactgacttctacagcagcagacagcatgccactaatgtggggattatgtttagagacaaggaaaatgccctcatgccaaattggctgcacctgcctgtgggctaccacggcagagcctcatcagtggtggtgtcaggcacaccaattaggaggccaatgggacagatgaagcctgacgactcaaagccaccagtctatggcgcctgcaaactgctggacatggagctggagatggctttctttgtggggcctggcaacaggctgggagaaccaatccctatttcaaaggcccacgagcacatttttggcatggtgctcatgaatgactggtctgccagagatatccagaagtgggaatacgtgcccctgggcccttttctgggcaagagctttggcaccacagtgtcaccttgggtggtcccaatggatgccctgatgccctttgccgtgcccaaccccaagcaggatcccagaccactgccctacctgtgccacgatgagccctatacctttgacatcaacctgtcagtcaacctgaagggggagggcatgagccaggccgccactatttgcaagagcaactttaaatatatgtactggactatgctccaacagctcactcaccactcagtgaatggctgcaatctgaggcctggcgacctcctggctagcggcactatctctggccctgaacctgagaactttgggagcatgctggagctgtcatggaaagggacaaagcctattgacctggggaatggccagacaaggaagtttctcctggatggcgatgaggtcatcatcactgggtactgccagggcgatggctacaggattggatttggacagtgtgctggcaaagtgctcccagctctgctgccctca

SEQ ID NO: 8 - DNA, 3102 bp, Artificial Sequence
  Synthetic Human truncated ATP7B
cctgagcaggagagacagatcacagccagagaaggggccagtcggaaaatcttatctaagctttctttgcctacccgtgcctgggaaccagcaatgaagaagagttttgcttttgacaatgttggctatgaaggtggtctggatggcctgggcccttcttctcagccgcagaagtgcttcttacagatcaaaggcatgacctgtgcatcctgtgtgtctaacatagaaaggaatctgcagaaagaagctggtgttctctccgtgttggttgccttgatggcaggaaaggcagagatcaagtatgacccagaggtcatccagcccctcgagatagctcagttcatccaggacctgggttttgaggcagcagtcatggaggactacgcaggctccgatggcaacattgagctgacaatcacagggatgacctgcgcgtcctgtgtccacaacatagagtccaaactcacgaggacaaatggcatcacttatgcctccgttgcccttgccaccagcaaagcccttgttaagtttgacccggaaattatcggtccacgggatattatcaaaattattgaggaaattggctttcatgcttccctggcccagagaaaccccaacgctcatcacttggaccacaagatggaaataaagcagtggaagaagtctttcctgtgcagcctggtgtttggcatccctgtcatggccttaatgatctatatgctgatacccagcaacgagccccaccagtccatggtcctggaccacaacatcattccaggactgtccattctaaatctcatcttctttatcttgtgtacctttgtccagctcctcggtgggtggtacttctacgttcaggcctacaaatctctgagacacaggtcagccaacatggacgtgctcatcgtcctggccacaagcattgcttatgtttattctctggtcatcctggtggttgctgtggctgagaaggcggagaggagccctgtgacattcttcgacacgccccccatgctctttgtgttcattgccctgggccggtggctggaacacttggcaaagagcaaaacctcagaagccctggctaaactcatgtctctccaagccacagaagccaccgttgtgacccttggtgaggacaatttaatcatcagggaggagcaagtccccatggagctggtgcagcggggcgatatcgtcaaggtggtccctgggggaaagtttccagtggatgggaaagtcctggaaggcaataccatggctgatgagtccctcatcacaggagaagccatgccagtcactaagaaacccggaagcactgtaattgcggggtctataaatgcacatggctctgtgctcattaaagctacccacgtgggcaatgacaccactttggctcagattgtgaaactggtggaagaggctcagatgtcaaaggcacccattcagcagctggctgaccggtttagtggatattttgtcccatttatcatcatcatgtcaactttgacgttggtggtatggattgtaatcggttttatcgattttggtgttgttcagagatactttcctaaccccaacaagcacatctcccagacagaggtgatcatccggtttgctttccagacgtccatcacggtgctgtgcattgcctgcccctgctccctggggctggccacgcccacggctgtcatggtgggcaccggggtggccgcgcagaacggcatcctcatcaagggaggcaagcccctggagatggcgcacaagataaagactgtgatgtttgacaagactggcaccattacccatggcgtccccagggtcatgcgggtgctcctgctgggggatgtggccacactgcccctcaggaaggttctggctgtggtggggactgcggaggccagcagtgaacaccccttgggcgtggcagtcaccaaatactgtaaagaggaacttggaacagagaccttgggatactgcacggacttccaggcagtgccaggctgtggaattgggtgcaaagtcagcaacgtggaaggcatcctggcccacagtgagcgccctttgagtgcaccggccagtcacctgaatgaggctggcagccttcccgcagaaaaagatgcagtcccccagaccttctctgtgctgattggaaaccgtgagtggctgaggcgcaacggtttaaccatttctagcgatgtcagtgacgctatgacagaccacgagatgaaaggacagacagccatcctggtggctattgacggtgtgctctgtgggatgatcgcaatcgcagacgctgtcaagcaggaggctgccctggctgtgcacacgctgcagagcatgggtgtggacgtggttctgatcacgggggacaaccggaagacagccagagctattgccacccaggttggcatcaacaaagtctttgcagaggtgctgccttcgcacaaggtggccaaggtccaggagctccagaataaagggaagaaagtcgccatggtgggggatggggtcaatgactccccggccttggcccaggcagacatgggtgtggccattggcaccggcacggatgtggccatcgaggcagccgacgtcgtccttatcagaaatgatttgctggatgtggtggctagcattcacctttccaagaggactgtccgaaggatacgcatcaacctggtcctggcactgatttataacctggttgggatacccattgcagcaggtgtcttcatgcccatcggcattgtgctgcagccctggatgggctcagcggccatggcagcctcctctgtgtctgtggtgctctcatccctgcagctcaagtgctataagaagcctgacctggagaggtatgaggcacaggcgcatggccacatgaagcccctgacggcatcccaggtcagtgtgcacataggcatggatgacaggtggcgggactcccccagggccacaccatgggaccaggtcagctatgtcagccaggtgtcgctgtcctccctgacgtccgacaagccatctcggcacagcgctgcagcagacgatgatggggacaagtggtctctgctcctgaatggcagggatgaggagcagtacatc

SEQ ID NO: 9 - DNA, 19 bp, Artificial Sequence
  Synthetic primer
atgttccacgaagaagcca

SEQ ID NO: 10 - DNA, 19 bp, Artificial Sequence
  Synthetic primer
tcagcaggctgaaattggt

SEQ ID NO: 11 - DNA, 23 bp, Artificial Sequence
  Synthetic primer
agctgtttcttactccattctca

SEQ ID NO: 12 - DNA, 24 bp, Artificial Sequence
  Synthetic probe
aggcaacgtcatgggtgtgacttt